(19) United States
(12) Patent Application Publication
(10) Pub. No.: US 2013/0281397 A1
(43) Pub. Date: Oct. 24, 2013

(54) TREATMENT OF DISEASES BY EPIGENETIC REGULATION

(71) Applicants: Kevin G. McLure, Calgary (CA); Peter Ronald Young, San Francisco, CA (US)

(72) Inventors: Kevin G. McLure, Calgary (CA); Peter Ronald Young, San Francisco, CA (US)

(73) Assignee: RVX Therapeutics Inc., Calgary (CA)

(21) Appl. No.: 13/832,484

(22) Filed: Mar. 15, 2013

Related U.S. Application Data
(60) Provisional application No. 61/635,726, filed on Apr. 19, 2012.

(51) Int. Cl. A61K 31/472; A61K 31/517; A61K 31/5415; A61K 31/496; A61K 31/695; A61K 31/519; A61K 45/06; A61K 31/4375; A61K 31/5377

(52) U.S. Cl. CPC A61K 31/472; A61K 31/4375; A61K 31/517; A61K 31/5415; A61K 31/5377; A61K 31/695; A61K 31/519; A61K 45/06; A61K 31/496
USPC 514/43; 514/309; 514/300; 514/266.31; 514/226.5; 514/235.2; 514/234.5; 514/63; 514/264.1; 514/64; 514/171; 514/110; 514/291; 514/253.05; 514/266.21; 514/266.24; 514/252.17; 514/266.2

(57) ABSTRACT

The present disclosure provides non-naturally occurring polyphenol compounds that inhibit the bromodomain and extra terminal domain (BET) proteins. The disclosed compositions and methods can be used for treatment and prevention of cancer, including NUT midline carcinoma, Burkitt's Lymphoma, Acute Myelogenous Leukemia, and Multiple Myeloma; autoimmune or inflammatory diseases or conditions, and sepsis.

The present disclosure relates to methods of treating and/or preventing diseases or disorders that respond to BET (bromodomain and extra terminal domain protein) inhibitors, such as cancer.

Cancer is a group of diseases caused by dysregulated cell proliferation. Therapeutic approaches aim to decrease the numbers of cancer cells by inhibiting cell replication or by inducing cancer cell differentiation or death, but there is still significant unmet medical need for more efficacious therapeutic agents. Cancer cells accumulate genetic and epigenetic changes that alter cell growth and metabolism in order to promote cell proliferation and increased resistance to programmed cell death, or apoptosis. Some of these changes include inactivation of tumor suppressor genes, activation of oncogenes, as well as modifications of the regulation of chromatin structure. Watson, Cancer Discovery 1:477-480 (2011); Morin et al., Nature 476:298-303 (2011).

Many modifications of histones in chromatin have been characterized, including acetylation at multiple lysines in histones H3 and H4. Peserico and Simone, J. Biomed. Biotechnol. 2011:371832 (2011). Histone acetylation is controlled by acetylases (HATs) as well as deacetylases (HDACs), and small molecule HDAC inhibitors have been developed with cancer as an indication. Hoshino and Matsubara, Surg. Today 40:809-815 (2010). Histone acetylation controls gene expression by recruiting protein complexes that bind directly to acetylated lysine via bromodomains. Sanchez and Zhou, Curr. Opin. Drug Discov. Devel. 12(5):659-665 (2009). One such family, the bromodomain and extra terminal domain (BET) proteins, comprises Brd2, Brd3, Brd4, and BrdT each of which contains two bromodomains in tandem that can independently bind to acetylated lysines. Wu and Chiang, J. Biol. Chem. 282(18):13141-13145 (2007). BET proteins exert some of their effects on transcription by recruiting the positive transcription elongation factor b (p-TEFb), which stimulates transcription elongation by phosphorylating the C-terminal domain of RNA polymerase II and results in increased expression of growth promoting genes, such as, for example, c-Myc and the well established cancer target Aurora B. Filippakopoulos et al., Nature 468:1067-1073 (2010).

Molecules that bind to BET proteins and prevent them from binding to chromatin, inhibit transcription and prevent cell replication, which is useful in cancer therapy and other settings. For example, it has been shown that BET proteins can be displaced from the chromatin by small molecule inhibitors, such as, for example, JQ1, I-BET, and I-BET151, which specifically compete with the acetyl-lysine binding pocket of the BET protein bromodomains thereby preventing transcription elongation of their target genes. Filippakopoulos et al. (2010); Nicodeme et al., Nature 468:1119-1123 (2010); Dawson et al., Nature 478:529-533 (2011).

Inhibition of BET bromodomain-promoter interactions results in a subsequent reduction of myc transcription and protein levels. This results in G₁ arrest and extensive apoptosis in a variety of leukemia and lymphoma cell lines. Mertz et al., PNAS 108(40):16669-16674 (2011). The Myc family of proto-oncogenes (c-myc, l-myc, n-myc) is activated in 25-35% of all human cancers. Vita and Henrickson, Seminars in Cancer Biol. 16:318-330 (2006). Mouse models of cancer driven by overexpression of c-myc demonstrate that transiently inhibiting c-myc expression can cause tumor regression, cell death, and in some cancers such as leukemia, complete disease remission. Soucek et al., Nature 455:679-683 (2008). The absence of a clear ligand-binding domain of c-myc has made the development of an inhibitor a formidable challenge, thus alternative strategies to targeting c-myc transcription must be developed. Delmore et al., Cell 146:904-917 (2011). A mouse model of aggressive human medulloblastoma, in which c-myc is overexpressed, suggests that BET inhibitors may be useful for treating myc-amplified medulloblastoma. Kawauchi et al., Cancer Cell 21:168-180 (2012); Pei et al., Cancer Cell 21:155-167 (2012). Similarly, inhibition of n-myc through RNA interference significantly reduced tumor growth in neuroblastoma mouse models. Jiang et al., Biochem. Biophs. Res. Commun. 410:364-370 (2011). A similar role for l-myc was suggested in small cell lung carcinoma cell lines using antisense oligonucleotides to inhibit l-myc amplification. Dosaka-Akita et al., Cancer Res. 55:1559-1564 (1995). Therefore BET inhibitors have potential to be efficacious in treating multiple types of cancer.

In fact, small molecules that target the bromodomains of BET family members have demonstrated potential therapeutic use in treating cancer. See, for example, Dawson et al. (2011), showing that a small molecule inhibitor of the BET family has a profound efficacy against human and murine mixed lineage leukemia (MLL)-fusion cell lines by early cell cycle arrest and apoptosis. Its mechanism of efficacy is the selective abrogation of Brd3/4 recruitment to chromatin. BET inhibitor JQ1 has demonstrated potent antitumor activity in murine xenograft models of NUT (nuclear protein in testis) midline carcinoma (NMC), a rare but lethal form of cancer. NMC tumor cell growth is driven by a translocation of the Brd4 gene to the nutlin 1 gene. Filippakopoulos et al., (2010). JQ1 was also shown to be a potent antiproliferator in multiple myeloma, associated with cell cycle arrest and cellular senescence. Delmore et al. (2011).

BET inhibitors are also expected to be potential therapeutics for other types of cancer. For example, in acute myeloid leukemia (AML), Brd4 is required to sustain myc expression and continued disease progression. Zuber et al., Nature 478:524-8 (2011). Moreover, inactivation of Brd4 results in a rapid and drastic down-regulation of the transcription of the proto-oncogenes c-myc and n-myc in cell lines they are amplified. Dawson et al. (2011); Delmore et al. (2011); Zuber et al. (2011); Mertz et al. (2011). Consequently, treatment of tumors that have activation of c-myc with a BET inhibitor resulted in tumor regression through inactivation of c-myc transcription. BET inhibitors are also expected to have application in multiple myeloma, as the multiple myeloma SET domain (MMSET) which is implicated in this disease also binds to BET proteins. Dawson et al. (2011).

In addition to cancer, BET inhibitors are also expected to have anti-inflammatory and immunomodulatory properties. Lamotte et al., Bioorganic & Med. Chem. Letters (Feb. 24, 2012); Prinjha et al., Trends Pharmacol. Sci. 33(3):146-153 (2012). BET inhibitors I-BET and I-BET151 decrease IL-6 expression in vivo. I-BET was shown to confer protection against lipopolysaccharide-induced endotoxic shock and bacteria-induced sepsis and I-BET151 was shown to suppress bacterial-induced inflammation and sepsis in a murine model. Nicodeme et al. (2010); Lamotte et al. (2012). In addition, BET inhibitors may modulate responses to viral and bacterial infections, including HIV, herpes, and papilloma viruses.

### DETAILED DESCRIPTION

The present invention provides methods of treating and/or preventing cancer and other diseases by administering a compound that inhibits BET family proteins. Cancers that may be treated or prevented with the methods of the invention include cancers that are sensitive to a compound that binds to bromodomains of BET family proteins, including NUT midline carcinoma; cancers that exhibit c-myc overexpression, including, but not limited to, Burkitt's lymphoma, acute myelogenous leukemia, multiple myeloma, aggressive human medulloblastoma; cancers overexpressing n-myc; and cancers that rely on the recruitment of p-TEFb to regulate activated oncogenes such as, for example, NOTCH1. In some embodiments, BET inhibitors may induce apoptosis in cancer cells by decreasing expression of the anti-apoptosis gene Bcl2. In certain embodiments, the methods of the invention are used to treat or prevent cancers, including hematological, epithelial including lung, breast and colon carcinomas, midline carcinomas, mesenchymal, hepatic, renal and neurological tumours.

The methods of invention include administering to a mammal, such as a human, for the purpose of treating or preventing cancer or other diseases that respond to BET inhibitors, a therapeutically effective amount of at least one compound of Formula I:

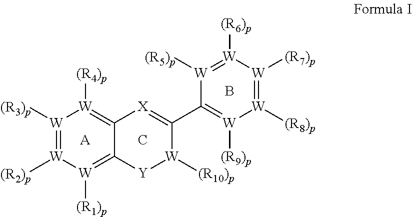

wherein:

X is selected from CR₁₁ and N;

Y is selected from CO and SO₂;

R₁, R₂, R₃, R₄, R₅, R₆, R₇, R₈, R₉, R₁₀, and R₁₁ are each independently selected from alkoxy, aryloxy, alkyl, amide, amino, aryl, arylalkyl, carbamate, carboxy, cycloalkyl, halogen, haloalkyl, heteroaryl, heterocyclyl, hydrogen, hydroxyl, ketone, phosphate, sulfide, sulfinyl, sulfonyl, sulfonic acid, sulfonamide and thioketone; and

each W is independently selected from C and N, wherein if W is N, then p is 0 and if W is C, then p is 1;

or tautomer, stereoisomer, pharmaceutically acceptable salt or hydrate thereof.

In certain embodiments, the method for treating or preventing cancer or other diseases that respond to BET inhibitors, comprises administering a therapeutically effective amount of at least one compound of Formula II to a mammal in need thereof:

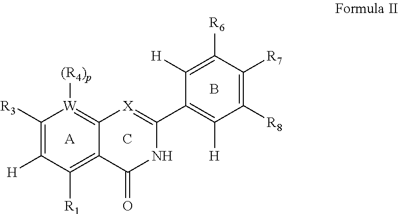

wherein:

X is selected from CH and N;

R₁ and R₃ are each independently selected from alkoxy (preferably methoxy), alkyl, halogen (preferably chloride);

R₄ is H;

R₆ and R₈ are each independently selected from alkoxy, alkyl (preferably methyl), halogen (preferably chloride or fluoride), hydrogen;

R₇ is selected from alkoxy, alkyl, amino, ether, hydrogen, and hydroxyl; and

W is selected from C and N, wherein if W is N, then p is 0 or 1, and if W is C, then p is 1;

or tautomer, stereoisomer, pharmaceutically acceptable salt or hydrate thereof.

In certain embodiments, the methods of the invention are useful for the prevention or treatment of diseases that benefit from increased cell death or differentiation, or decreased cell proliferation. This may occur by, for example, decreased expression of a Myc family member or an oncogene required for tumor growth, or increase of a tumor suppressor gene, the latter antagonized by BET proteins. The method of the invention can be used to increase cancer cell death or decrease cancer cell proliferation, including, for example, by decreasing expression of Myc family member. Decreasing expression of the Myc family member may refer to, but is not limited to, transcriptionally modulating the expression of its gene or genes that have been either amplified in the genome or translocated to another chromosomal location, or transcriptionally altered in order to increase its expression (i.e. overexpression) thereby affecting the level of the c-myc protein produced. A decrease in the Myc family member mRNA levels may decrease proliferation of cancer cells and/or increase cancer cell death, including but not limited to apoptosis.

In other embodiments, the methods of the invention are useful for the prevention or treatment of diseases such as cancer in combination with other drugs. In some embodiments, at least one compound of Formula I or Formula II or tautomer, stereoisomer, pharmaceutically acceptable salt, or hydrate thereof may be administered in combination with a standard of care drug(s) for any given tumor type, including, but not limited to, bortezomib, thalidomide, dexamethasone, 5-azacitidine, decitabine, vorinostat, or cyclophosphamide in multiple myeloma. In another embodiment, a compound of Formula I or Formula II or tautomers, stereoisomers, pharmaceutically acceptable salts, or hydrates of compounds of Formula I or Formula II may be administered in combination with a PI3K or mTOR inhibitor such as rapamycin or a rapamycin analog. Similarly, a compound of Formula I or Formula II could be administered in combination with gamma secretase inhibitors which inhibit NOTCH1 (given the relationship between c-myc and NOTCH1) or AMPK inducers such as metformin or phenformin for leukemia. Another example of a potentially useful combination is combining a BET inhibitor which decreases myc expression, with an ornithine decarboxylase inhibitor such as difluoromethylornithine that inhibits a myc target.

In certain embodiments, the methods of the invention provide treatment of autoimmune and inflammatory diseases or conditions by administering at least one compound of Formula I or Formula II or tautomer, stereoisomer, pharmaceutically acceptable salt, or hydrate thereof. In other embodiments, compounds of Formula I or Formula II or tautomers, stereoisomers, pharmaceutically acceptable salts, or hydrates thereof, may be employed to treat other diseases caused by bacterial or viral infection, such as, for example, infection by HIV, HPV, or herpes virus. Certain embodiments of the invention provide, for use of a compound of Formula I or Formula II or tautomers, stereoisomers, pharmaceutically acceptable salts, or hydrates thereof, in the manufacture of a medicament for the treatment of cancer, autoimmune and inflammatory diseases or conditions, AIDS, or sepsis.

### DEFINITIONS

As used in the present specification, the following words, phrases and symbols are generally intended to have the meanings as set forth below, except to the extent that the context in which they are used indicates otherwise. The following abbreviations and terms have the indicated meanings throughout.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, for example, stabilization of a discernible symptom, physiologically, for example, stabilization of a physical parameter, or both. In yet another embodiment, “treatment” or “treating” refers to delaying the onset of a disease or disorder.

As used herein, “inhibiting” refers to blocking, suppressing, or in any other way, reducing, the biological function of a BET protein in a subject.

As used herein, “reducing” refers to reducing the overall levels of BET biological activity, for example, by inhibiting the availability of the level of BET protein in the body for other biological interactions.

The term “autoimmune and inflammatory diseases or conditions” as used herein refers to a wide variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, dry eye, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, Celiac disease, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs.

The term “autoimmune and inflammatory diseases or conditions” is also intended to include acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement and acute rejection of transplanted organs. The term “autoimmune and inflammatory diseases or conditions” is also intended to include diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, postsurgical syndromes, sarcoidosis, Herxheimer reactions, encephalitis, myelitis, meningitis, malaria, and SIRS associated with viral infections such as influenza, herpes zoster, herpes simplex, and coronavirus.

As used herein, the term “effective amount” means that amount of a compound of Formula I or Formula II or tautomer, stereoisomer, pharmaceutically acceptable salt, or hydrate thereof, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “prevention” or “preventing” refers to a reduction of the risk of acquiring a given disease or disorder.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “aldehyde” or “formyl” as used herein refers to —CHO.

The term “alkenyl” as used herein refers to an unsaturated straight or branched hydrocarbon having at least one carbon-carbon double bond, such as a straight or branched group of 2-22, 2-8, or 2-6 carbon atoms, referred to herein as (C₂-C₂₂)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, and (C₁-C₆)alkoxy, respectively. Exemplary alkoxy groups include, but are not limited to methoxy, ethoxy, etc.

The term “alkyl” as used herein refers to a saturated straight or branched hydrocarbon, such as a straight or branched group of 1-22, 1-8, or 1-6 carbon atoms, referred to herein as (C₁-C₂₂)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkynyl” as used herein refers to an unsaturated straight or branched hydrocarbon having at least one carbon-carbon triple bond, such as a straight or branched group of 2-22, 2-8, or 2-6 carbon atoms, referred to herein as (C₂-C₂₂)alkynyl, (C₂-C₈)alkynyl, and (C₂-C₆)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” as used herein refers to the form —NR₉C(O)R_{b}, or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through R_{b} or R_{c}. The amide also may be cyclic, for example R_{b} and R_{c} may be joined to form a 3- to 12-membered ring, such as a 3- to 10-membered ring or a 5- to 6-membered ring. The term “amide” encompasses groups such as sulfonamide, urea (ureido), carbamate, carbamic acid, and cyclic versions thereof. The term “amide” also encompasses an amide group attached to a carboxy group, for example, -amide-COOH or salts such as -amide-COONa, etc, an amino group attached to a carboxy group, for example, -amino-COON or salts such as -amino-COONa, etc.

The term “amine” or “amino” as used herein refers to the form —NR_{d}Rₑ or —N(R_{d})Rₑ— where R_{d} and Rₑ are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example, R_{d} and Rₑ may be joined together or with the N to form a 3- to 12-membered ring, for example, morpholino or piperidinyl. The term amino also includes the corresponding quaternary ammonium salt of any amino group. Exemplary amino groups include alkyl amino groups, wherein at least one of R_{d} and Rₑ is an alkyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “arylalkyl” as used herein refers to an alkyl group having at least one aryl substituent, for example -aryl-alkyl-. Exemplary arylalkyl groups include, but are not limited to, arylalkyls having a monocyclic aromatic ring system, wherein the ring comprises 6 carbon atoms, referred to herein as “(C₆)arylalkyl.”

The term “aryloxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.”

The term “arylthio” as used herein refers to an aryl group attached to an sulfur atom. Exemplary arylthio groups include, but are not limited to, arylthios having a monocyclic aromatic ring system, wherein the ring comprises 6 carbon atoms, referred to herein as “(C₆)arylthio.”

The term “arylsulfonyl” as used herein refers to an aryl group attached to a sulfonyl group, for example, —S(O)₂-aryl-. Exemplary arylsulfonyl groups include, but are not limited to, arylsulfonyls having a monocyclic aromatic ring system, wherein the ring comprises 6 carbon atoms, referred to herein as “(C₆)arylsulfonyl.”

The term “benzyl” as used herein refers to the group —CH₂-phenyl.

The term “bicyclic aryl” as used herein refers to an aryl group fused to another aromatic or non-aromatic carbocylic or heterocyclic ring. Exemplary bicyclic aryl groups include, but are not limited to, naphthyl or partly reduced forms thereof, such as di-, tetra-, or hexahydronaphthyl.

The term “bicyclic heteroaryl” as used herein refers to a heteroaryl group fused to another aromatic or non-aromatic carbocylic or heterocyclic ring. Exemplary bicyclic heteroaryls include, but are not limited to, 5, 6 or 6,6-fused systems wherein one or both rings contain heteroatoms. The term “bicyclic heteroaryl” also encompasses reduced or partly reduced forms of fused aromatic system wherein one or both rings contain ring heteroatoms. The ring system may contain up to three heteroatoms, independently selected from oxygen, nitrogen, or sulfur. The bicyclic system may be optionally substituted with one or more groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary bicyclic heteroaryls include, but are not limited to, quinazolinyl, benzothiophenyl, benzoxazolyl, benzimidazolyl, benzothiazolyl, benzofuranyl, indolyl, quinolinyl, isoquinolinyl, phthalazinyl, benzotriazolyl, benzopyridinyl, and benzofuranyl.

The term “carbamate” as used herein refers to the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates, for example, wherein at least one of R_{g}, Rₕ and Rᵢ are independently selected from aryl or heteroaryl, such as pyridine, pyridazine, pyrimidine, and pyrazine.

The term “carbonyl” as used herein refers to —C(O)—.

The term “carboxy” as used herein refers to —COON or its corresponding carboxylate salts, for example —COONa, etc. The term carboxy also includes “carboxycarbonyl,” for example, a carboxy group attached to a carbonyl group, for example, —C(O)—COOH or salts such as —C(O)—COONa, etc.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides, etc., for example, succinic anhydride, succinimide, etc.

The term “ester” refers to the structure —O(O)O—, —C(O)O—Rⱼ—, —RₖC(O)O—Rⱼ—, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rj or Rk is alkyl, such as —O—C(O)-alkyl-, —C(O)—O-alkyl-, -alkyl-C(O)—O-alkyl-, etc. Exemplary esters also include aryl or heteroaryl esters, for example wherein at least one of Rj or Rk is a heteroaryl group such as pyridine, pyridazine, pyrimidine and pyrazine, such as a nicotinate ester. Exemplary esters also include reverse esters having the structure —RₖC(O)O—, where the oxygen is bound to the parent molecular group. Exemplary reverse esters include succinate, D-argininate, L-argininate, L-lysinate and D-lysinate. Esters also include carboxylic acid anhydrides and acid halides.

The term “ether” refers to the structure —Rₗ—Rₘ—, where Rₗ and Rₘ can independently be alkyl, alkenyl, alkynyl, aryl, cycloalkyl, heterocyclyl, or ether. The ether can be attached to the parent molecular group through Rₗ or Rₘ. Exemplary ethers include, but are not limited to, alkoxyalkyl and alkoxyaryl groups. Ethers also includes polyethers, for example, where one or both of Rₗ and Rₘ are ethers.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example one to three heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2 to 5 carbon atoms and 1 to 3 heteroatoms, referred to herein as “(C₂-C₅)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refers to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rₙ— (such as acetyl, —C(O)CH₃) or —Rₙ—C(O)—Rₒ—. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic acid and maleic acid.

The term “nitro” as used herein refers to the structure —NO₂.

The term “perfluoroalkoxy” as used herein refers to an alkoxy group in which all of the hydrogen atoms have been replaced by fluorine atoms.

The term “perfluoroalkyl” as used herein refers to an alkyl group in which all of the hydrogen atoms have been replaced by fluorine atoms. Exemplary perfluoroalkyl groups include, but are not limited to, (C₁₋₅) perfluoroalkyl, such as trifluoromethyl, etc.

The term “perfluorocycloalkyl” as used herein refers to a cycloalkyl group in which all of the hydrogen atoms have been replaced by fluorine atoms.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

The term “phosphate” as used herein refers to the structure —OP(O)O₂—, —RₓOP(O)O₂—, —OP(O)O₂R_{y}—, or —RₓOP(O)O₂R_{y}—, wherein Rₓ and R_{y} can be selected from alkyl, alkenyl, alkynyl, aryl, cycloalkyl, heterocyclyl, and hydrogen.

The term “sulfide” as used herein refers to the structure —R_{z}S—, where R_{z} can be selected from alkyl, alkenyl, alkynyl, aryl, arylalkyl, cycloalkyl, haloalkyl, heteroaryl, and heterocyclyl. The sulfide may be cyclic, forming a 3-12 membered ring. The term “alkylsulfide” as used herein refers to an alkyl group attached to a sulfur atom.

The term “sulfinyl” as used herein refers to the structure —S(O)O—, —RₚS(O)O—, —RₚS(O)OR_{q}—, or —S(O)OR_{q}—, wherein Rₚ and R_{q} can be selected from alkyl, alkenyl, aryl, arylalkyl, cycloalkyl, haloalkyl, heteroaryl, heterocyclyl, and hydroxyl. Exemplary sulfinyl groups include, but are not limited to, alkylsulfinyls wherein at least one of Rₚ or R_{q} is alkyl, alkenyl or alkynyl.

The term “sulfonamide” as used herein refers to the structure —(Rᵣ)—N—S(O)₂—Rₛ— or —Rₜ(Rᵣ)—N—S(O)₂—Rₛ, where Rₜ, Rᵣ, and Rₛ can be, for example, hydrogen, alkyl, alkenyl, alkynyl, aryl, cycloalkyl, and heterocyclyl. Exemplary sulfonamides include alkylsulfonamides (for example, where Rₛ is alkyl), arylsulfonamides (for example, where Rₛ is aryl), cycloalkyl sulfonamides (for example, where Rₛ is cycloalkyl), and heterocyclyl sulfonamides (for example, where Rₛ is heterocyclyl), etc.

The term “sulfonate” as used herein refers to —OSO₃⁻. Sulfonate includes salts such as —OSO₃Na, —OSO₃K, etc. and the acid —OSO₃H

The term “sulfonic acid” refers to —SO₃H— and its corresponding salts, for example —SO₃K—, —SO₃Na—.

The term “sulfonyl” as used herein refers to the structure RᵤSO₂—, where Rᵤ can be alkyl, alkenyl, alkynyl, aryl, cycloalkyl, and heterocyclyl, for example, alkylsulfonyl. The term “alkylsulfonyl” as used herein refers to an alkyl group attached to a sulfonyl group. “Alkylsulfonyl” groups can optionally contain alkenyl or alkynyl groups.

The term “thioketone” refers to the structure —Rᵥ—C(S)—R_{w}—. The ketone can be attached to another group through Rᵥ or R_{w}. Rᵥ or R_{w} can be alkyl, alkenyl, alkynyl, cycloalkyl, heterocyclyl or aryl, or Rᵥ and R_{w} can be joined to form a 3- to 12-membered ring.

“Alkyl,” “alkenyl,” “alkynyl,” “alkoxy,” “amino,” and “amide” groups can be substituted with or interrupted by or branched with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, thioketone, ureido, and nitrogen. The substituents may be branched to form a substituted or unsubstituted heterocycle or cycloalkyl.

As used herein, a “suitable substituent” refers to a group that does not nullify the synthetic or pharmaceutical utility of the compounds of the invention or the intermediates useful for preparing them. Examples of suitable substituents include, but are not limited to: C₁₋₂₂, C₁₋₈, and C₁₋₆ alkyl, alkenyl or alkynyl; C₁₋₆ aryl, C₂₋₅ heteroaryl; C₃₋₇ cycloalkyl; C₁₋₂₂, C₁₋₈, and C₁₋₆ alkoxy; C₆ aryloxy; —CN; —OH; oxo; halo, carboxy; amino, such as —NH(C₁₋₂₂, C₁₋₈, or C₁₋₆ alkyl), —N(C₁₋₂₂, C₁₋₈, and C₁₋₆ alkyl)₂, —NH((C₆)aryl), or —N((C₆)aryl)₂; formyl; ketones, such as —CO(C₁₋₂₂, C₁₋₈, and C₁₋₆ alkyl), —CO((C₆ aryl) esters, such as —CO₂(C₁₋₂₂, C₁₋₈, and C₁₋₆ alkyl) and —CO₂ (C₆ aryl). One of skill in art can readily choose a suitable substituent based on the stability and pharmacological and synthetic activity of the compound of the invention.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention. A discussion is provided in Higuchi et al., “Pro-drugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

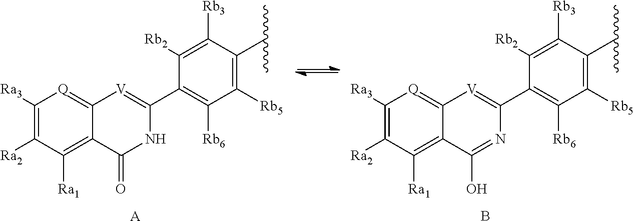

### Embodiments of the Invention

One embodiment of the invention provides a method for treating or preventing diseases or disorders that respond to BET inhibitors comprising administering to a mammal, such as a human, a therapeutically effective amount of a compound of Formula I:

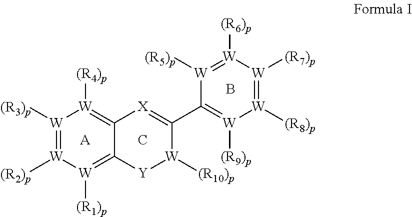

wherein:

X is selected from CR₁₁ and N;

Y is selected from CO and SO₂;

R₁, R₂, R₃, R₄, R₅, R₆, R₇, R₈, R₉, R₁₀, and R₁₁ are each independently selected from alkoxy, aryloxy, alkyl, amide, amino, aryl, arylalkyl, carbamate, carboxy, cycloalkyl, halogen, haloalkyl, heteroaryl, heterocyclyl, hydrogen, hydroxyl, ketone, phosphate, sulfide, sulfinyl, sulfonyl, sulfonic acid, sulfonamide and thioketone; and

each W is independently selected from C and N, wherein if W is N, then p is 0 and if W is C, then p is 1;

or tautomer, stereoisomer, pharmaceutically acceptable salt, or hydrate thereof.

One embodiment of the invention provides a method treating or preventing diseases or disorders that respond to BET inhibitors in a mammal, such as a human, comprising administering a therapeutically effective amount of a compound of Formula II:

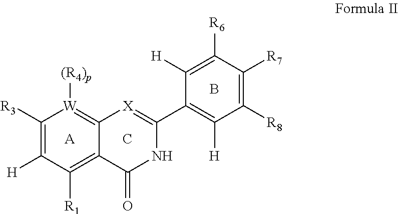

wherein:

X is selected from CH and N;

R₁ and R₃ are each independently selected from alkoxy (preferably methoxy), alkyl, halogen (preferably chloride);

R₄ is H;

R₆ and R₈ are each independently selected from alkoxy, alkyl (preferably methyl), halogen (preferably chloride or fluoride), hydrogen;

R₇ is selected from alkoxy, alkyl, amino, ether, hydrogen, and hydroxyl; and

W is selected from C and N, wherein if W is N, then p is 0 or 1, and if W is C, then p is 1;

or tautomer, stereoisomer, pharmaceutically acceptable salt, or hydrate thereof.

In some embodiments of Formula II, R₇ is not diethylamino or an alkoxy substituted with a carboxylate group; or the compound of Formula II is a tautomer, stereoisomer, pharmaceutically acceptable salt, or hydrate thereof.

In certain embodiments of Formula I: for W—(R₁₀)ₚ, W is N and p is 1; or the compound of Formula I is a tautomer, stereoisomer, pharmaceutically acceptable salt, or hydrate thereof.

In certain embodiments of Formula I: X is CR₁₁; and for W—(R₁₀)ₚ, W is N and p is 0; or the compound of Formula I is a tautomer, stereoisomer, pharmaceutically acceptable salt, or hydrate thereof.

In certain embodiments of Formula I: Y is SO₂; and for W—(R₁₀)ₚ, W is N, and p is 0; or the compound of Formula I is a tautomer, stereoisomer, pharmaceutically acceptable salt, or hydrate thereof.

In certain embodiments of Formula I: R₁ and R₃ are each independently an alkoxy; or the compound of Formula I is a tautomer, stereoisomer, pharmaceutically acceptable salt, or hydrate thereof.

In certain embodiments of Formula I: R₁ and R₃ are each independently an alkoxy; R₆ and R₈ are each independently selected from alkyl and hydrogen; R₇ is selected from amino and alkoxy; and X is N; or the compound of Formula I is a tautomer, stereoisomer, pharmaceutically acceptable salt, or hydrate thereof.

In certain embodiments of Formula I: R₁ and R₃ are each independently an alkoxy; R₆ and R₈ are each independently selected from alkyl and hydrogen; R₇ is selected from amino, hydroxyl, and alkoxy; X is CR₁₁; Y is CO; and for W—(R₁₀)ₚ, W is N and R₁₀ is hydrogen; or the compound of Formula I is a tautomer, stereoisomer, pharmaceutically acceptable salt, or hydrate thereof.

In certain embodiments of Formula I: R₁ and R₃ are each independently an alkoxy; R₆ and R₈ are each independently selected from alkyl and hydrogen; and R₇ is selected from amino and alkoxy; X is N; Y is CO; and for W—(R₁₀)ₚ, W is N and R₁₀ is hydrogen; or the compound of Formula I is a tautomer, stereoisomer, pharmaceutically acceptable salt, or hydrate thereof.

In certain embodiments of Formula I: R₅ and R₉ are each hydrogen; or the compound of Formula I is a tautomer, stereoisomer, pharmaceutically acceptable salt, or hydrate thereof.

In certain embodiments of Formula I: at least one of R₁, R₂, and R₃ is not hydrogen; or the compound of Formula I is a tautomer, stereoisomer, pharmaceutically acceptable salt, or hydrate thereof.

In certain embodiments of Formula I: at least one of R₁, R₂, and R₃ is not hydrogen; R₆ and R₈ are each independently selected from alkyl and hydrogen; R₇ is selected from amino and alkoxy, and X is N; or the compound of Formula I is a tautomer, stereoisomer, pharmaceutically acceptable salt, or hydrate thereof.

In certain embodiments of Formula I: at least one of R₁, R₂, and R₃ is not hydrogen; R₆ and R₈ are each independently selected from alkyl and hydrogen; R₇ is selected from amino, hydroxyl, and alkoxy; X is CR₁₁; Y is CO; and for W—(R₁₀)ₚ, W is N and R₁₀ is hydrogen; or the compound of Formula I is a tautomer, stereoisomer, pharmaceutically acceptable salt, or hydrate thereof.

In certain embodiments of Formula I: at least one of R₁, R₂, and R₃ is not hydrogen; R₆ and R₈ are each independently selected from alkyl and hydrogen; R₇ is selected from amino and alkoxy; X is N; Y is CO; and for W—(R₁₀)ₚ, W is N and R₁₀ is hydrogen; or the compound of Formula I is a tautomer, stereoisomer, pharmaceutically acceptable salt, or hydrate thereof.

In certain embodiments of Formula I: at least two of R₆, R₇, and R₈ are not hydrogen; or the compound of Formula I is a tautomer, stereoisomer, pharmaceutically acceptable salt, or hydrate thereof.

In certain embodiments of Formula I: at least two of R₆, R₇, and R₈ are not hydrogen; R₆ and R₈ are each independently selected from alkyl and hydrogen; R₇ is selected from amino and alkoxy, and X is N; or the compound of Formula I is a tautomer, stereoisomer, pharmaceutically acceptable salt, or hydrate thereof.

In certain embodiments of Formula I: at least two of R₆, R₇, and R₈ are not hydrogen; R₆ and R₈ are each independently selected from alkyl and hydrogen; R₇ is selected from amino, hydroxyl, and alkoxy; X is CR₁₁; Y is CO; and for W—(R₁₀)ₚ, W is N; and R₁₀ is hydrogen; or the compound of Formula I is a tautomer, stereoisomer, pharmaceutically acceptable salt, or hydrate thereof.

In certain embodiments of Formula I: at least two of R₆, R₇, and R₈ are not hydrogen; R₆ and R₈ are each independently selected from alkyl and hydrogen; R₇ is selected from amino and alkoxy; X is N; Y is CO; and for W—(R₁₀)ₚ, W is N; and R₁₀ is hydrogen; or the compound of Formula I is a tautomer, stereoisomer, pharmaceutically acceptable salt, or hydrate thereof.

In certain embodiments of Formula I: X is selected from CH and N; or the compound of Formula I is a tautomer, stereoisomer, pharmaceutically acceptable salt, or hydrate thereof.

In certain embodiments of Formula I: X is selected from CH and N; and for W—(R₁₀)ₚ, W is N and p is 1; or the compound of Formula I is a tautomer, stereoisomer, pharmaceutically acceptable salt, or hydrate thereof.

In some embodiments of Formula I, R₇ is an amino or an alkoxy selected from the group represented by Formula III:

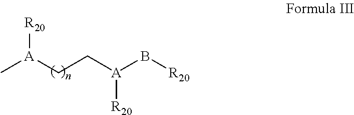

wherein:

A is selected from O and N;

n is selected from 0, 1, 2, and 3;

B is selected from —C(O)N(Rₕ)₂—, —S(O)₂N(Rₕ)₂—, —C(O)—, —S(O)₂—, and —C(O)O—, wherein each Rₕ is independently selected from alkyl, aryl, arylalkyl, cycloalkyl, haloalkyl, heteroaryl, heterocyclyl, and hydrogen; and

R₂₀ is selected from C₁-C₆ alkyl, C₁-C₆ alkenyl, C₁-C₆ alkynyl, aryl, arylalkyl, cycloalkyl, haloalkyl, heteroaryl, heterocyclyl, and hydrogen.

In some embodiment of Formula III: if A is O and B is —C(O)N(Rₕ)₂—, then R₂₀ is not an unsaturated cycloalkyl.

In certain embodiments of Formula I:

X is selected from CR₁₁, and N,

Y is selected from CO and SO₂,

R₁ and R₃ are each independently selected from alkoxy (preferably methoxy), alkyl, amino, halogen (preferably chloride), and hydrogen;

R₂ is selected from alkoxy, alkyl, alkenyl, amide, amino, halogen (preferably bromide or chloride), and hydrogen;

R₄ is H;

R₅ and R₉ are each independently selected from halogen (preferably chloride) and hydrogen;

R₆ and R₈ are each independently selected from alkoxy, alkyl (preferably methyl), amino, halogen (preferably chloride and fluoride), and hydrogen;

R₇ is selected from alkoxy, alkyl, alkenyl, amide, amino, ether, hydrogen, and hydroxyl;

R₁₀ is selected from hydrogen and alkyl (preferably methyl);

R₁₁ is selected from hydrogen, unsubstituted alkyl (preferably C₁₋₃ alkyl), unsubstituted alkenyl (preferably C₁₋₃ alkenyl), and unsubstituted alkynyl (preferably C₁₋₃ alkynyl); or

two adjacent substituents selected from R₁, R₂, R₃, R₆, R₇, R₈, R₁₀, and R₁₁ are connected to form a group selected from aryl, heteroaryl, cycloalkyl, and heterocyclyl; and

W is selected from C and N, wherein if W is N, then p is 0 or 1, and if W is C, then p is 1;

or the compound of Formula I is a tautomer, stereoisomer, pharmaceutically acceptable salt or hydrate thereof.

In certain embodiments of Formula I:

X is N,

Y is selected from CO and SO₂,

R₁ and R₃ are each independently selected from alkoxy (preferably methoxy), alkyl, amino, halogen (preferably chloride), and hydrogen;

R₂ is selected from alkoxy, alkyl, alkenyl, amide, amino, halogen (preferably bromide or chloride), and hydrogen;

R₄ is H;

R₅ and R₉ are each independently selected from halogen (preferably chloride) and hydrogen;

R₆ and R₈ are each independently selected from alkoxy, alkyl (preferably methyl), amino, halogen (preferably chloride and fluoride), and hydrogen;

R₇ is selected from alkoxy, alkyl, alkenyl, amide, amino, ether, hydrogen, and hydroxyl;

R₁₀ is selected from hydrogen and alkyl (preferably methyl);

R₁₁ is selected from hydrogen, unsubstituted alkyl (preferably C₁₋₃ alkyl), unsubstituted alkenyl (preferably C₁₋₃ alkenyl), and unsubstituted alkynyl (preferably C₁₋₃ alkynyl); or

two adjacent substituents selected from R₁, R₂, R₃, R₆, R₇, R₈, R₁₀, and R₁₁ are connected to form a group selected from aryl, heteroaryl, cycloalkyl, and heterocyclyl; and

W is selected from C and N, wherein if W is N, then p is 0 or 1, and if W is C, then p is 1;

or the compound of Formula I is a tautomer, stereoisomer, pharmaceutically acceptable salt or hydrate thereof.

In certain embodiments of Formula I:

X is selected from N and CH;

Y is CO;

R₁ and R₃ are each independently selected from alkoxy and hydrogen;

R₂ is selected from alkoxy, alkyl, and hydrogen;

R₄ is H;

R₅ and R₉ are each hydrogen;

R₆ and R₈ are each independently selected from alkyl, alkoxy, chloride, and hydrogen;

R₇ is selected from amino, hydroxyl, alkoxy (preferably a substituted ethoxy group), and alkyl substituted with a heterocyclyl;

R₁₀ is hydrogen; or

two adjacent substituents selected from R₆, R₇, and R₈ are connected to form a heterocyclyl;

each W is independently selected from C and N, wherein if W is N, then p is 0 or 1, and if W is C, then p is 1; and

for W—(R₁₀)ₚ, W is N and p is 1;

with the provisio that at least one of R₁ and R₃ is alkoxy;

with the proviso that if R₇ is selected from hydroxyl and alkoxy, then at least one of R₆ and R₈ are independently selected from alkyl, alkoxy, and chloride;

with the proviso that if R₇ is an amino, then X is N;

with the proviso that if for W—(R₇)ₚ, W is N and p is 0, then at least one of R₆ and R₈ is selected from alkyl, alkoxy, and chloride;

or the compound of Formula I is a tautomer, stereoisomer, pharmaceutically acceptable salt, or hydrate thereof.

In certain embodiments of Formula II: R₁ and R₃ are each independently an alkoxy; or the compound of Formula II is a tautomer, stereoisomer, pharmaceutically acceptable salt, or hydrate thereof.

In certain embodiments of Formula II: R₁ and R₃ are each independently an alkoxy; R₆ and R₈ are each independently selected from alkyl and hydrogen; R₇ is selected from amino and alkoxy; and X is N; or the compound of Formula II is a tautomer, stereoisomer, pharmaceutically acceptable salt, or hydrate thereof.

In certain embodiments of Formula II: R₁ and R₃ are each independently an alkoxy; R₆ and R₈ are each independently selected from alkyl and hydrogen; R₇ is selected from amino, hydroxyl, and alkoxy; and X is CH; or the compound of Formula II is a tautomer, stereoisomer, pharmaceutically acceptable salt, or hydrate thereof.

In certain embodiments of Formula II: R₆ and R₈ are each independently selected from alkyl and hydrogen; R₇ is selected from amino and alkoxy; and X is N; or the compound of Formula II is a tautomer, stereoisomer, pharmaceutically acceptable salt, or hydrate thereof.

In certain embodiments of Formula II: R₆ and R₈ are each independently selected from alkyl and hydrogen; R₇ is selected from amino, hydroxyl, and alkoxy; and X is CH; or the compound of Formula II is a tautomer, stereoisomer, pharmaceutically acceptable salt, or hydrate thereof.

In certain embodiments of Formula II: R₇ is selected from alkoxy, alkyl, amino, and hydroxyl; or the compound of Formula II is a tautomer, stereoisomer, pharmaceutically acceptable salt, or hydrate thereof.

In some embodiments of Formula II:

X is N;

R₁ and R₃ are each independently selected from alkoxy;

R₄ is H;

R₆ and R₈ are each independently selected from alkyl, alkoxy, and hydrogen;

R₇ is selected from amino, alkoxy (preferably a substituted ethoxy group), and alkyl substituted with a heterocyclyl;

W is selected from C and N, wherein if W is N, then p is 0 or 1, and if W is C, then p is 1;

with the proviso that if R₇ is alkoxy, then at least one of R₆ and R₈ are independently selected from alkyl and alkoxy;

or the compound of Formula II is a tautomer, stereoisomer, pharmaceutically acceptable salt or hydrate thereof.

In certain embodiments of Formula II: at least two of R₆, R₇, and R₈ are not hydrogen; or the compound of Formula II is a tautomer, stereoisomer, pharmaceutically acceptable salt, or hydrate thereof.

In certain embodiments of Formula II: at least two of R₆, R₇, and R₈ are not hydrogen; R₆ and R₈ are each independently selected from alkyl and hydrogen; and R₇ is selected from amino and alkoxy, and X is N; or the compound of Formula II is a tautomer, stereoisomer, pharmaceutically acceptable salt, or hydrate thereof.

In certain embodiments of Formula II: at least two of R₆, R₇, and R₈ are not hydrogen; R₆ and R₈ are each independently selected from alkyl and hydrogen; R₇ is selected from amino, hydroxyl, and alkoxy; X is CH; or the compound of Formula II is a tautomer, stereoisomer, pharmaceutically acceptable salt, or hydrate thereof.

In some embodiments of Formula II: X is CH; R₁ and R₃ are independently alkoxy; R₆ and R₈ are alkyl; and R₇ is alkoxy; or the compound of Formula II is a tautomer, stereoisomer, pharmaceutically acceptable salt, or hydrate thereof.

In some embodiments of Formula II: X is N; R₁ and R₃ are independently alkoxy; R₆ and R₈ are alkyl; and R₇ is alkoxy; or the compound of Formula II is a tautomer, stereoisomer, pharmaceutically acceptable salt, or hydrate thereof.

In some embodiments of Formula II: X is CH; R₁ and R₃ are independently alkoxy; R₆ and R₈ are alkyl; and R₇ is alkoxy substituted with a hydroxy; or the compound of Formula II is a tautomer, stereoisomer, pharmaceutically acceptable salt, or hydrate thereof.

In some embodiments of Formula II: X is N; R₁ and R₃ are independently alkoxy: R₆ and R₈ are alkyl; and R₇ is alkoxy substituted with a hydroxy; or the compound of Formula II is a tautomer, stereoisomer, pharmaceutically acceptable salt, or hydrate thereof.

In some embodiments of Formula II: X is CH; R₁ and R₃ are independently alkoxy; R₆ and R₈ are alkyl; and R₇ is alkoxy substituted with an amino; or the compound of Formula II is a tautomer, stereoisomer, pharmaceutically acceptable salt, or hydrate thereof.

In some embodiments of Formula II: X is N; R₁ and R₃ are independently alkoxy; R₆ and R₈ are alkyl; and R₇ is alkoxy substituted with an amino; or the compound of Formula II is a tautomer, stereoisomer, pharmaceutically acceptable salt, or hydrate thereof.

In some embodiments of Formula II: X is CH; R₁ and R₃ are independently alkoxy; R₆ and R₈ are alkyl; and R₇ is alkoxy substituted with a heterocycle; or the compound of Formula II is a tautomer, stereoisomer, pharmaceutically acceptable salt, or hydrate thereof.

In some embodiments of Formula II: X is N; R₁ and R₃ are independently alkoxy; R₆ and R₈ are alkyl; and R₇ is alkoxy substituted with a heterocycle; or the compound of Formula II is a tautomer, stereoisomer, pharmaceutically acceptable salt, or hydrate thereof.

In some embodiments of Formula II: X is CH; R₁ and R₃ are independently alkoxy; R₆ is hydrogen; and R₈ is selected from alkyl, halogen, and alkoxy; and R₇ is hydroxy; or the compound of Formula II is a tautomer, stereoisomer, pharmaceutically acceptable salt, or hydrate thereof.

In some embodiments of Formula II: X is N; R₁ and R₃ are alkoxy; R₆ is hydrogen; and R₈ is selected from alkyl, halogen, and alkoxy; and R₇ is hydroxy; or the compound of Formula II is a tautomer, stereoisomer, pharmaceutically acceptable salt, or hydrate thereof.

In some embodiments of Formula II: X is CH, R₁ and R₃ are alkoxy; R₆ is hydrogen; and R₈ is selected from alkyl, halogen, and alkoxy; and R₇ is alkoxy; or the compound of Formula II is a tautomer, stereoisomer, pharmaceutically acceptable salt, or hydrate thereof.

In some embodiments of Formula II: X is N; R₁ and R₃ are independently alkoxy; R₆ is hydrogen; and R₈ is selected from alkyl, halogen, and alkoxy; and R₇ is alkoxy; or the compound of Formula II is a tautomer, stereoisomer, pharmaceutically acceptable salt, or hydrate thereof.

In some embodiments of Formula II: X is CH; R₁ and R₃ are independently alkoxy; R₆ is hydrogen; and R₈ is selected from alkyl, halogen, and alkoxy; and R₇ is alkoxy substituted with a hydroxy; or the compound of Formula II is a tautomer, stereoisomer, pharmaceutically acceptable salt, or hydrate thereof.

In some embodiments of Formula II: X is N; R₁ and R₃ are independently alkoxy; R₆ is hydrogen; and R₈ is selected from alkyl, halogen, and alkoxy; and R₇ is alkoxy substituted with a hydroxy; or the compound of Formula II is a tautomer, stereoisomer, pharmaceutically acceptable salt, or hydrate thereof.

In some embodiments of Formula II: X is CH; R₁ and R₃ are independently alkoxy; R₆ is hydrogen; and R₈ is selected from alkyl, halogen, and alkoxy; and R₇ is alkoxy substituted with an amino; or the compound of Formula II is a tautomer, stereoisomer, pharmaceutically acceptable salt, or hydrate thereof.

In some embodiments, the compound of Formula II is selected from:
2-(4-(2-hydroxyethoxy)-3,5-dimethylphenyl)-5,7-dimethoxyquinazolin-4(3H)-one (Example 16);
N-(2-(4-(5,7-dimethoxy-4-oxo-3,4-dihydroquinazolin-2-yl)-2,6-dimethylphenoxy)ethyl)methanesulfonamide (Example 96);
2-(4-hydroxy-3-methylphenyl)-5,7-dimethoxyquinazolin-4(3H)-one (Example 92);
2-(4-(2-hydroxyethoxy)-3-methylphenyl)-5,7-dimethoxyquinazolin-4(3H)-one (Example 91);
2-(4-(bis(2-hydroxyethyl)amino)phenyl)-5,7-dimethoxyquinazolin-4(3H)-one (Example 41);
2-(3,5-dimethyl-4-(2-morpholinoethoxy)phenyl)-5,7-dimethoxyquinazolin-4(3H)-one (Example 17);
2-(3,5-dimethyl-4-(2-(pyrrolidin-1-yl)ethoxy)phenyl)-5,7-dimethoxyquinazolin-4(3H)-one;
3-(4-hydroxy-3,5-dimethylphenyl)-6,8-dimethoxy-7-(morpholinomethyl)isoquinolin-1(2H)-one (Example 46);
   and stereoisomers, tautomers, pharmaceutically acceptable salts, and hydrates thereof.

In certain embodiments of Formula II: R₇ is an alkoxy group substituted with an amino; or the compound of Formula II is a tautomer, stereoisomer, pharmaceutically acceptable salt, or hydrate thereof.

In some embodiments of Formula II: R₇ is an alkoxy substituted with a cyclic amine; or the compound of Formula II is a tautomer, stereoisomer, pharmaceutically acceptable salt, or hydrate thereof.

In some embodiments the compound of Formula II is 2-(3,5-dimethyl-4-(2-(pyrrolidin-1-yl)ethoxy)phenyl)-5,7-dimethoxyquinazolin-4(3H)-one.

In some embodiments of Formula II: R₇ is an amino or an alkoxy selected from the group represented by Formula III:

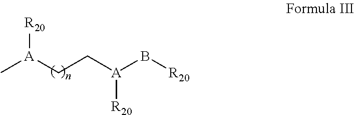

wherein:

A is selected from O and N;

n is selected from 0, 1, 2, and 3;

B is selected from —C(O)N(Rₕ)₂—, —S(O)₂N(Rₕ)₂—, —C(O)—, —S(O)₂—, and —C(O)O—, wherein each Rₕ is independently selected from alkyl, aryl, arylalkyl, cycloalkyl, haloalkyl, heteroaryl, heterocyclyl, and hydrogen; and

R₂₀ is selected from C₁-C₆ alkyl, C₁-C₆ alkenyl, C₁-C₆ alkynyl, aryl, arylalkyl, cycloalkyl, haloalkyl, heteroaryl, heterocyclyl, and hydrogen.

In some embodiments of Formula III: if A is O and B is —C(O)N(Rₕ)₂—, then R₂₀ is not an unsaturated cycloalkyl; or the compound of Formula II is a tautomer, stereoisomer, pharmaceutically acceptable salt, or hydrate thereof.

In some embodiments, the compound of Formula II is selected from:
2-(4-(5,7-dimethoxy-4-oxo-3,4-dihydroquinazolin-2-yl)-2,6-dimethylphenoxy)ethyl cyclohexylcarbamate (Example 102);
N-(2-(4-(5,7-dimethoxy-4-oxo-3,4-dihydroquinazolin-2-yl)-2,6-dimethylphenoxy)ethyl)acetamide (Example 106);
N-(2-(4-(5,7-dimethoxy-4-oxo-3,4-dihydroquinazolin-2-yl)-2,6-dimethylphenoxy)ethyl)isobutyramide (Example 108);
1-(2-(4-(5,7-dimethoxy-4-oxo-3,4-dihydroquinazolin-2-yl)-2,6-dimethylphenoxy)ethyl)-3-phenylurea (Example 111);
3-(2-(4-(5,7-dimethoxy-4-oxo-3,4-dihydroquinazolin-2-yl)-2,6-dimethylphenoxy)ethyl)-1,1-dimethylurea (Example 112);
   and stereoisomers, tautomers, pharmaceutically acceptable salts, and hydrates thereof.

In certain embodiments of Formula II: two adjacent substituents selected from R₆, R₇, and R₈ are connected to form a group selected from aryl, heteroaryl, cycloalkyl, and heterocyclyl.

In certain embodiments of Formula II:

X is selected from N and CH;

R₁ and R₃ are each independently selected from alkoxy and hydrogen;

R₆ and R₈ are each independently selected from alkyl, alkoxy, chloride, and hydrogen;

R₇ is selected from amino, hydroxyl, alkoxy (preferably a substituted ethoxy group), and alkyl substituted with a heterocyclyl;

two adjacent substituents selected from R₆, R₇, and R₈ are connected to form a heterocyclyl;

R₄ is H;

W is selected from C and N, wherein if W is N, then p is 0 or 1, and if W is C, then p is 1;

with the provisio that at least one of R₁ and R₃ is alkoxy;

with the proviso that if R₇ is hydroxyl or alkoxy, then at least one of R₆ and R₈ are independently selected from alkyl, alkoxy, and chloride;

with the proviso that if R₇ is an amino, then X is N;

or the compound of Formula II is a tautomer, stereoisomer, pharmaceutically acceptable salt, or hydrate thereof.

In some embodiments, the compound of Formula II is selected from:
3-(4-hydroxy-3,5-dimethylphenyl)-6,8-dimethoxyisoquinolin-1(2H)-one (Example 4);
3-(4-(2-hydroxyethoxy)-3,5-dimethylphenyl)-6,8-dimethoxyisoquinolin-1(2H)-one (Example 6);
3-(4-hydroxy-3,5-dimethylphenyl)-7-(morpholinomethyl)isoquinolin-1(2H)-one (Example 8);
2-(4-hydroxy-3,5-dimethylphenyl)-5,7-dimethoxyquinazolin-4(3H)-one (Example 10);
3-(4-(2-hydroxy-2-methylpropoxy)-3,5-dimethylphenyl)-6,8-dimethoxyisoquinolin-1(2H)-one (Example 11);
7-(4-hydroxy-3,5-dimethylphenyl)-2,4-dimethoxy-1,6-naphthyridin-5(6H)-one (Example 13);
2-(4-(2-hydroxyethoxy)-3,5-dimethylphenyl)-5,7-dimethoxyquinazolin-4(3H)-one (Example 16);
3-(3,5-dimethyl-4-(2-(4-methylpiperazin-1-yl)ethoxy)phenyl)-6,8-dimethoxyisoquinolin-1(2H)-one (Example 19);
2-(4-hydroxy-3-methoxyphenyl)-5,7-dimethoxyquinazolin-4(3H)-one (Example 26);
2-(4-(bis(2-hydroxyethyl)amino)phenyl)-5,7-dimethoxyquinazolin-4(3H)-one (Example 41);
2-(4-(bis(2-hydroxyethyl)amino)phenyl)-6,7-dimethoxyquinazolin-4(3H)-one (Example 42);
2-(2,3-dihydrobenzo[b][1,4]dioxin-6-yl)-6,7-dimethoxyquinazolin-4(3H)-one (Example 43);
2-(4-((4-ethylpiperazin-1-yl)methyl)phenyl)-5,7-dimethoxyquinazolin-4(3H)-one (Example 62);
2-(4-(2-hydroxyethoxy)-3,5-dimethylphenyl)-5,7-dimethoxypyrido[2,3-d]pyrimidin-4(3H)-one (Example 65);
2-(2-chloro-6-methylpyridin-4-yl)-5,7-dimethoxyquinazolin-4(3H)-one (Example 83);
5,7-dimethoxy-2-(4-methoxy-3,5-dimethylphenyl)quinazolin-4(3H)-one (Example 84);
2-(4-amino-3,5-dimethylphenyl)-5,7-dimethoxyquinazolin-4(3H)-one (Example 85);
N1-(2-(4-(5,7-dimethoxy-4-oxo-3,4-dihydroquinazolin-2-yl)-2,6-dimethylphenoxy)ethyl)-N2-methylphthalamide (Example 93);
2-(4-(2-aminoethoxy)-3,5-dimethylphenyl)-5,7-dimethoxyquinazolin-4(3H)-one (Example 93);
4-chloro-N-(2-(4-(5,7-dimethoxy-4-oxo-3,4-dihydroquinazolin-2-yl)-2,6-dimethylphenoxy)ethyl)benzenesulfonamide (Example 95);

and stereoisomers, tautomers, pharmaceutically acceptable salts, and hydrates thereof.

In some embodiments, the compound of Formula II is selected from:
3-(4-Hydroxyphenyl)-2H-isoquinolin-1-one;
4-(1-Oxo-1,2-dihydroisoquinolin-3-yl)phenyl 2-amino-5-guanidinopentanoate trihydrochloride;
3-(4-hydroxyphenyl)-6,8-dimethoxyisoquinolin-1(2H)-one, 3-(4-hydroxy-3,5-dimethylphenyl)-6,8-dimethoxy-2-methylisoquinolin-1(2H)-one;
3-(4-hydroxy-3,5-dimethylphenyl)-6,8-dimethoxyisoquinolin-1(2H)-one;
7-(4-hydroxy-3,5-dimethylphenyl)-1,6-naphthyridin-5(6H)-one, 3-(4-(2-hydroxyethoxy)-3,5-dimethylphenyl)-6,8-dimethoxyisoquinolin-1(2H)-one;
3-(4-(2-(dimethylamino)ethoxy)-3,5-dimethylphenyl)-6,8-dimethoxyisoquinolin-1(2H)-one;
3-(4-hydroxy-3,5-dimethylphenyl)-7-(morpholinomethyl)isoquinolin-1(2H)-one;
2-hydroxy-7-(4-hydroxy-3,5-dimethylphenyl)-4-methoxy-1,6-naphthyridin-5(6H)-one;
2-(4-hydroxy-3,5-dimethylphenyl)-5,7-dimethoxyquinazolin-4(3H)-one;
3-(4-(2-hydroxy-2-methylpropoxy)-3,5-dimethylphenyl)-6,8-dimethoxyisoquinolin-1(2H)-one;
6,8-dimethoxy-3-(4-hydroxy-3,5-dimethylphenyl)-2H-1,2-benzothiazine-1,1-dioxide;
7-(4-hydroxy-3,5-dimethylphenyl)-2,4-dimethoxy-1,6-naphthyridin-5(6H)-one;
3-(4-hydroxy-3,5-dimethylphenyl)-6,8-dimethoxy-2,7-dimethylisoquinolin-1(2H)-one;
3-(4-hydroxy-3,5-dimethylphenyl)-6,8-dimethoxy-2-methyl-7-(morpholinomethyl)isoquinolin-1(2H)-one;
3-(3,5-dimethyl-4-(2-morpholinoethoxy)phenyl)-6,8-dimethoxyisoquinolin-1(2H)-one;
2-(4-(2-hydroxyethoxy)-3,5-dimethylphenyl)-5,7-dimethoxyquinazolin-4(3H)-one;
2-(3,5-dimethyl-4-(2-morpholinoethoxy)phenyl)-5,7-dimethoxyquinazolin-4(3H)-one;
2-(3,5-dimethyl-4-(2-(pyrrolidin-1-yl)ethoxy)phenyl)-5,7-dimethoxyquinazolin-4(3H)-one
3-(3,5-dimethyl-4-(2-(4-methylpiperazin-1-yl)ethoxy)phenyl)-6,8-dimethoxyisoquinolin-1(2H)-one;
2-(4-hydroxyphenyl)-5,7-dimethoxyquinazolin-4(3H)-one;
2-(4-(5,7-dimethoxy-4-oxo-3,4-dihydroquinazolin-2-yl)phenoxy)acetic acid;
5,7-dimethoxy-2-(pyridin-2-yl)quinazolin-4(3H)-one;
5,7-dimethoxy-2-(pyridin-3-yl)quinazolin-4(3H)-one;
2-(3,5-di-tert-butyl-4-hydroxyphenyl)-5,7-dimethoxyquinazolin-4(3H)-one;
2-(3,5-dimethoxyphenyl)-5,7-dimethoxyquinazolin-4(3H)-one;
5,7-dimethoxy-2-(4-methoxyphenyl)quinazolin-4(3H)-one;
2-(4-hydroxy-3-methoxyphenyl)-5,7-dimethoxyquinazolin-4(3H)-one;
2-(3-chloro-4-hydroxyphenyl)-5,7-dimethoxyquinazolin-4(3H)-one;
5,7-dimethoxy-2-(pyridin-4-yl)quinazolin-4(3H)-one;
2-(3,5-dimethyl-4-(2-morpholinoethoxy)phenyl)quinazolin-4(3H)-one;
2-(4-(2-hydroxyethoxy)-3,5-dimethylphenyl)quinazolin-4(3H)-one;
2-(2,3-dihydrobenzo[b][1,4]dioxin-6-yl)quinazolin-4(3H)-one;
2-(4-(dimethylamino)naphthalen-1-yl)-5,7-dimethoxyquinazolin-4(3H)-one;
2-(4-(5,7-dimethoxy-4-oxo-3,4-dihydroquinazolin-2-yl)phenoxy)acetamide;
2-(4-(4-oxo-3,4-dihydroquinazolin-2-yl)phenoxy)acetic acid;
2-(4-(dimethylamino)pyridinon-1-yl)quinazolin-4(3H)-one;
2-(4-(4-oxo-3,4-dihydroquinazolin-2-yl)phenoxy)acetamide;
2-(2,3-dihydrobenzo[b][1,4]dioxin-6-yl)-5,7-dimethoxyquinazolin-4(3H)-one;
5,7-dimethoxy-2-(4-(4-methylpiperazin-1-yl)phenyl)quinazolin-4(3H)-one;
2-(4-(dimethylamino)pyridinon-1-yl)-6,7-dimethoxyquinazolin-4(3H)-one;
2-(4-(bis(2-hydroxyethyl)amino)phenyl)quinazolin-4(3H)-one;
2-(4-(bis(2-hydroxyethyl)amino)phenyl)-5,7-dimethoxyquinazolin-4(3H)-one;
2-(4-(bis(2-hydroxyethyl)amino)phenyl)-6,7-dimethoxyquinazolin-4(3H)-one;
2-(2,3-dihydrobenzo[b][1,4]dioxin-6-yl)-6,7-dimethoxyquinazolin-4(3H)-one;
5,7-dimethoxy-2-(4-morpholinophenyl)quinazolin-4(3H)-one;
7-(3,5-dimethyl-4-(2-morpholinoethoxy)phenyl)-2,4-dimethoxy-1,6-naphthyridin-5(6H)-one;
3-(4-hydroxy-3,5-dimethylphenyl)-6,8-dimethoxy-7-(morpholinomethyl)isoquinolin-1(2H)-one;
2-(4-hydroxy-3,5-dimethylphenyl)-6,7-dimethoxyquinazolin-4(3H)-one;
3-(4-(5,7-dimethoxy-4-oxo-3,4-dihydroquinazolin-2-yl)phenyl)propanoic acid;
N-(2-(4-hydroxy-3,5-dimethylphenyl)-4-oxo-3,4-dihydroquinazolin-6-yl)acetamide;
2-(4-(6,7-dimethoxy-4-oxo-3,4-dihydroquinazolin-2-yl)-2,6-dimethylphenoxy)acetamide;
2-(3-chloro-4-(2-hydroxyethoxy)phenyl)-5,7-dimethoxyquinazolin-4(3H)-one;
2-(4-(2-hydroxyethoxy)-3-methoxyphenyl)-5,7-dimethoxyquinazolin-4(3H)-one;
2-(4-(2-hydroxyethoxy)-3,5-dimethylphenyl)-6,7-dimethoxyquinazolin-4(3H)-one;
5,7-dimethoxy-2-(4-((4-methylpiperazin-1-yl)methyl)phenyl)quinazolin-4(3H)-one;
N-(4-(5,7-dimethoxy-4-oxo-3,4-dihydroquinazolin-2-yl)-2,6-dimethylphenyl)-2-hydroxyacetamide;
7-(4-(2-hydroxyethoxy)-3,5-dimethylphenyl)-2,4-dimethoxy-1,6-naphthyridin-5(6H)-one;
2-(4-hydroxy-3,5-dimethylphenyl)-6-(morpholinomethyl)quinazolin-4(3H)-one;
2,4-dimethoxy-7-(4-methoxy-3,5-dimethylphenyl)-1,6-naphthyridin-5(6H)-one;
2-(4-(5,7-dimethoxy-4-oxo-3,4-dihydroquinazolin-2-yl)-2,6-dimethylphenoxy)acetic acid;
N-(4-(5,7-dimethoxy-4-oxo-3,4-dihydroquinazolin-2-yl)phenyl)-2-hydroxyacetamide;
5,7-dimethoxy-2-(4-(morpholinomethyl)phenyl)quinazolin-4(3H)-one;
2-(4-((4-ethylpiperazin-1-yl)methyl)phenyl)-5,7-dimethoxyquinazolin-4(3H)-one;
2-(4-hydroxy-3,5-dimethylphenyl)-5,7-dimethoxypyrido[2,3-d]pyrimidin-4(3H)-one;
5,7-dimethoxy-2-(4-methoxy-3-(morpholinomethyl)phenyl)quinazolin-4(3H)-one;
2-(4-(2-hydroxyethoxy)-3,5-dimethylphenyl)-5,7-dimethoxypyrido[2,3-d]pyrimidin-4(3H)-one;
2-(4-hydroxy-3,5-dimethylphenyl)-5,7-dimethoxy-1-methylquinazolin-4(1H)-one;
2-(3,5-dimethyl-4-(2-(pyrrolidin-1-yl)ethoxy)phenyl)-5,7-dimethoxyquinazolin-4(3H)-one;
N-(2-(4-(2-hydroxyethoxy)-3,5-dimethylphenyl)-4-oxo-3,4-dihydroquinazolin-6-yl)acetamide;
7-(4-hydroxy-3,5-dimethylphenyl)-2,4-diisopropoxy-1,6-naphthyridin-5(6H)-one;
2-(4-hydroxy-3-(2-hydroxyethyl)phenyl)-5,7-dimethoxyquinazolin-4(3H)-one;
2-(4-(2-hydroxyethoxy)-3,5-dimethylphenyl)-5,7-dimethylquinazolin-4(3H)-one;
2-(4-(bis(2-hydroxyethyl)amino)phenyl)-5,7-dimethoxypyrido[2,3-d]pyrimidin-4(3H)-one;
5,7-dimethoxy-2-(4-(2-methoxyethoxy)-3,5-dimethylphenyl)quinazolin-4(3H)-one;
5,7-dichloro-2-(4-(2-hydroxyethoxy)-3,5-dimethylphenyl)quinazolin-4(3H)-one;
2-(4-hydroxy-3,5-dimethylphenyl)-5,7-dimethoxy-6-(morpholinomethyl)quinazolin-4(3H)-one;
2-(4-(2-hydroxyethoxy)-3,5-dimethyl phenyl)-6-methoxyquinazolin-4(3H)-one;
2-(2-chlorophenyl)-5,7-dimethoxyquinazolin-4(3H)-one;
2-(4-(2-hydroxyethoxy)-3,5-dimethylphenyl)-5-methoxyquinazolin-4(3H)-one;
5,7-dimethoxy-2-o-tolylquinazolin-4(3H)-one;
5,7-dimethoxy-2-(6-(4-(methylsulfonyl)phenyl)pyridin-2-yl)quinazolin-4(3H)-one;
5,7-dimethoxy-2-(6-methylpyridin-2-yl)quinazolin-4(3H)-one;
5,7-dimethoxy-2-(6-(4-(methylthio)phenyl)pyridin-2-yl)quinazolin-4(3H)-one;
2-(2-chloro-6-methylpyridin-4-yl)-5,7-dimethoxyquinazolin-4(3H)-one;
5,7-dimethoxy-2-(4-methoxy-3,5-dimethylphenyl)quinazolin-4(3H)-one;
2-(4-amino-3,5-dimethylphenyl)-5,7-dimethoxyquinazolin-4(3H)-one;
5,7-dimethoxy-2-(1-phenyl-5-propyl-1H-pyrazol-4-yl)quinazolin-4(3H)-one;
2-(3,5-dimethylphenyl)-5,7-dimethoxyquinazolin-4(3H)-one;
2-(3-(2,6-dichlorophenyl)-5-methylisoxazol-4-yl)-5,7-dimethoxyquinazolin-4(3H)-one;
(E)-N′-(4-(5,7-dimethoxy-4-oxo-3,4-dihydroquinazolin-2-yl)-2,6-dimethylphenyl)-N,N-dimethylformimidamide;
6-bromo-2-(4-hydroxy-3,5-dimethylphenyl)quinazolin-4(3H)-one;
6-bromo-2-(4-(2-hydroxyethoxy)-3,5-dimethylphenyl)quinazolin-4(3H)-one;
6-bromo-2-(4-(2-(tert-butyldimethylsilyloxy)ethoxy)-3,5-dimethylphenyl)quinazolin-4(3H)-one;
2-(4-(benzyloxy)-3,5-dimethylphenyl)-5,7-dimethoxyquinazolin-4(3H)-one;
2-(4-(2-hydroxyethoxy)-3-methylphenyl)-5,7-dimethoxyquinazolin-4(3H)-one;
2-(4-hydroxy-3-methylphenyl)-5,7-dimethoxyquinazolin-4(3H)-one;
N1-(2-(4-(5,7-dimethoxy-4-oxo-3,4-dihydroquinazolin-2-yl)-2,6-dimethylphenoxy)ethyl)-N2-methylphthalamide;
2-(4-(2-aminoethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)-4-methoxybenzenesulfonamide;
4-chloro-N-(2-(4-(5,7-dimethoxy-4-oxo-3,4-dihydroquinazolin-2-yl)-2,6-dimethylphenoxy)ethyl)benzenesulfonamide;
N-(2-(4-(5,7-dimethoxy-4-oxo-3,4-dihydroquinazolin-2-yl)-2,6-dimethylphenoxy)ethyl)methanesulfonamide;
2-(4-(5,7-dimethoxy-4-oxo-3,4-dihydroquinazolin-2-yl)-2-methoxyphenoxy)acetic acid;
5-hydroxy-2-(4-hydroxy-3,5-dimethylphenyl)-7-methoxyquinazolin-4(3H)-one;
2-(4-(5,7-Dimethoxy-4-oxo-3,4-dihydroquinazolin-2-yl)-2,6-dimethyl-phenoxy)ethyl propylcarbamate;
2-(4-(5,7-Dimethoxy-4-oxo-3,4-dihydroquinazolin-2-yl)-2,6-dimethyl-phenoxy)ethyl methylcarbamate;
N-(2-(4-(5,7-Dimethoxy-4-oxo-3,4-dihydroquinazolin-2-yl)-2,6-dimethylphenoxy)ethyl)-4-methyl benzamide;
2-(4-(5,7-dimethoxy-4-oxo-3,4-dihydroquinazolin-2-yl)-2,6-dimethylphenoxy)ethyl cyclohexylcarbamate;
N-(2-(4-(5,7-dimethoxy-4-oxo-3,4-dihydroquinazolin-2-yl)-2,6-dimethylphenoxy)ethyl)benzenesulfonamide;
N-(2-(4-(5,7-dimethoxy-4-oxo-3,4-dihydroquinazolin-2-yl)-2,6-dimethylphenoxy)ethyl)-4-methylbenzenesulfonamide;
N-(2-(4-(5,7-dimethoxy-4-oxo-3,4-dihydroquinazolin-2-yl)-2,6-dimethylphenoxy)ethyl)-4-methoxybenzamide;
N-(2-(4-(5,7-dimethoxy-4-oxo-3,4-dihydroquinazolin-2-yl)-2,6-dimethylphenoxy)ethyl)acetamide;
N-(2-(4-(5,7-dimethoxy-4-oxo-3,4-dihydroquinazolin-2-yl)-2,6-dimethylphenoxy)ethyl)benzamide;
N-(2-(4-(5,7-dimethoxy-4-oxo-3,4-dihydroquinazolin-2-yl)-2,6-dimethylphenoxy)ethyl)isobutyramide;
1-(2-(4-(5,7-dimethoxy-4-oxo-3,4-dihydroquinazolin-2-yl)-2,6-dimethylphenoxy)ethyl)-3-methylurea;
1-(2-(4-(5,7-dimethoxy-4-oxo-3,4-dihydroquinazolin-2-yl)-2,6-dimethylphenoxy)ethyl)-3-(4-methoxyphenyl)urea;
1-(2-(4-(5,7-dimethoxy-4-oxo-3,4-dihydroquinazolin-2-yl)-2,6-dimethylphenoxy)ethyl)-3-phenylurea;
3-(2-(4-(5,7-dimethoxy-4-oxo-3,4-dihydroquinazolin-2-yl)-2,6-dimethylphenoxy)ethyl)-1,1-dimethylurea;

and stereoisomers, tautomers, pharmaceutically acceptable salts, and hydrates thereof.

In certain embodiments the therapeutically effective amount of the at least one compound of Formula I or Formula II, or tautomer, stereoisomer, pharmaceutically acceptable salt, or hydrate thereof, is administered with a pharmaceutically acceptable carrier in a pharmaceutically acceptable composition.

In certain embodiments of the method the disease or disorder is a cancer.

In some embodiments the cancer is a midline carcinoma.

In some embodiments the cancer is characterized by overexpression of c-myc.

In other embodiments the cancer is characterized by overexpression of l-myc.

In some embodiments the cancer is Burkitt's lymphoma, acute myelogenous leukemia, multiple myeloma, or aggressive human medulloblastoma.

In some embodiments the cancer is characterized by overexpression of n-myc.

In certain embodiments the cancer is selected from the group consisting of cancers that that rely on the recruitment of p-TEFb to regulate activated oncogenes such as, for example, NOTCH1.

In some embodiments of the method the compound of Formula I or Formula II or a tautomer, stereoisomer, pharmaceutically acceptable salt, or hydrate thereof induces apoptosis in cancer cells by decreasing expression of the anti-apoptosis gene Bcl2.

In certain embodiments the cancer is selected from the group consisting of hematological, epithelial including lung, breast and colon carcinomas, midline carcinomas, mesenchymal, hepatic, renal and neurological tumours.

The present disclosure provides a method of treating or preventing a disease or disorder in a mammal that benefits from increased cell death or differentiation, or decreased cell proliferation, comprising administering a compound of Formula I or Formula II, or a tautomer, stereoisomer, pharmaceutically acceptable salt, or hydrate thereof as defined above.

In some embodiments the compound of Formula I or Formula II or a tautomer, stereoisomer, pharmaceutically acceptable salt, or hydrate thereof is administered in combination with another anti-cancer agent.

In certain embodiments the anti-cancer agent is selected from the group consisting of bortezomib, thalidomide, dexamethasone, 5-azacitidine, decitabine, vorinostat, and cyclophosphamide.

In some embodiments the anti-cancer agent is a PI3K or mTOR inhibitor.

In other embodiments the anti-cancer agent is rapamycin or a rapamycin analog.

In certain embodiments the anti-cancer agent is a gamma secretase inhibitor.

In some embodiments the anti-cancer agent is an AMPK inducer.

In some embodiments the anti-cancer agent is metformin or phenformin.

In other embodiments the anti-cancer agent is an ornithine decarboxylase inhibitor.

In certain embodiments the anti-cancer agent is difluoromethylornithine.

A method is provided for treating or preventing autoimmune or inflammatory diseases or conditions comprising administering a therapeutically effective amount of a compound of Formula I or Formula II or a tautomer, stereoisomer, pharmaceutically acceptable salt, or hydrate thereof as defined above.

A method is provided for treating or preventing a disease or disorder caused by bacterial or viral infection comprising administering a therapeutically effective amount of a compound of Formula I or Formula II or a tautomer, stereoisomer, pharmaceutically acceptable salt, or hydrate thereof as defined above.

In some embodiments the disease or disorder is AIDS.

A method is provided for treating or preventing sepsis comprising administering a therapeutically effective amount of a compound of Formula I or Formula II or a tautomer, stereoisomer, pharmaceutically acceptable salt, or hydrate thereof as defined above.

### Pharmaceutical Compositions

Pharmaceutical compositions comprising at least one compound of Formula I or II, or tautomer, stereoisomer, pharmaceutically acceptable salt or hydrate thereof formulated together with one or more pharmaceutically acceptable carriers may be employed in the methods of the invention. These formulations include those suitable for oral, rectal, topical, intraocular, buccal and parenteral (for example, subcutaneous, intramuscular, intradermal, intravenous, or via implants) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of at least one compound of Formula I or II, or tautomer, stereoisomer, pharmaceutically acceptable salt or hydrate thereof as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of Formula I or II, or tautomer, stereoisomer, pharmaceutically acceptable salt or hydrate thereof as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations employed in the methods of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of Formula I or II, or tautomer, stereoisomer, pharmaceutically acceptable salt or hydrate thereof as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of Formula I or II, or tautomer, stereoisomer, pharmaceutically acceptable salt or hydrate thereof with a liquid or finely divided solid carrier, or both, and then, if necessary, shaping the product. For example, a tablet may be prepared by compressing or molding a powder or granules of at least one compound of Formula I or II, or tautomer, stereoisomer, pharmaceutically acceptable salt or hydrate thereof, which may be optionally combined with one or more accessory ingredients. Compressed tablets may be prepared by compressing, in a suitable machine, at least one compound of the invention in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the invention is moistened with an inert liquid diluent.

Formulations suitable for buccal (sub-lingual) administration include lozenges comprising at least one compound of Formula I or II, or tautomer, stereoisomer, pharmaceutically acceptable salt or hydrate thereof in a flavored base, usually sucrose and acacia or tragacanth, and pastilles comprising the at least one compound in an inert base such as gelatin and glycerin or sucrose and acacia.

Formulations of the invention suitable for parenteral administration comprise sterile aqueous preparations of at least one compound of Formula I or II, or tautomers, stereoisomers, pharmaceutically acceptable salts, and hydrates thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions employed in the methods of the invention may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula I or II, or tautomers, stereoisomers, pharmaceutically acceptable salts, and hydrates thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, for example, Freireich et al., Cancer Chemother. Reports 50(4):219-244 (1966) and Table 1 for Equivalent Surface Area Dosage Factors).

TABLE 1
Equivalent Surface Area Dosage Factors

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

In one embodiment, a compound of Formula I or II, or a tautomer, stereoisomer, pharmaceutically acceptable salt or hydrate thereof, is administered in combination with another therapeutic agent. The other therapeutic agent can provide additive or synergistic value relative to the administration of a compound of the invention alone. In certain embodiments, a compound of Formula I or II or a tautomer, stereoisomer, pharmaceutically acceptable salt or hydrate thereof, is administered in combination with one or more anti-cancer agents.

### Therapeutic Methods

The invention provides methods of treating or preventing diseases or disorders that respond to BET inhibitors, such as, for example, cancer, autoimmune and inflammatory diseases or conditions, and diseases caused by bacterial or viral infection, such as infection by HIV, HPV, or herpes virus. These methods comprise administering to a subject (for example, a mammal, such as a human) a therapeutically effective amount of at least one compound of Formula I or II, or a tautomer, stereoisomer, pharmaceutically acceptable salt or hydrate thereof. In another embodiment, at least one compound of Formula I or II, or tautomer, stereoisomer, pharmaceutically acceptable salt or hydrate thereof may be administered as a pharmaceutically acceptable composition, comprising one or more compounds of Formula I or II or tautomer, stereoisomer, pharmaceutically acceptable salt, or hydrate thereof and a pharmaceutically acceptable carrier.

In some embodiments, the disease or disorder is a cancer which may be treated or prevented by administering a therapeutically effective amount of at least one compound of Formula I or II, or tautomer, stereoisomer, pharmaceutically acceptable salt or hydrate thereof.

In certain embodiments, the cancer to be treated is a midline carcinoma. In some embodiments, the cancer is characterized by c-myc activation or overexpression. In other embodiments, the cancer is characterized by overexpression or activation of n-myc. In certain embodiments, the cancer is Burkitt's lymphoma, acute myelogenous leukemia, multiple myeloma, or aggressive human medulloblastoma. In some embodiments, the cancer relies on the recruitment of p-TEFb to regulate activated oncogenes such as, for example, NOTCH1. In some embodiments, the cancer to be treated or prevented by the methods of the invention is selected from the group consisting of hematological, epithelial including lung, breast and colon carcinomas, midline carcinomas, mesenchymal, hepatic, renal and neurological tumours.

The certain embodiments, administration of a compound of Formula I or Formula II or a tautomer, stereoisomer, pharmaceutically acceptable salt or hydrate thereof, to a mammal suffering from a cancer induces apoptosis in cancer cells by decreasing expression of the anti-apoptosis gene Bcl2. Thus, some embodiments of the invention provide a method of treating or preventing a disease or disorder in a mammal that benefits from increased cell death or differentiation, or decreased cell proliferation, comprising administering a compound of Formula I or Formula II or a tautomer, stereoisomer, pharmaceutically acceptable salt or hydrate thereof.

In some embodiments of the invention, the compound of Formula I or Formula II or a tautomer, stereoisomer, pharmaceutically acceptable salt or hydrate thereof is administered in combination with another anti-cancer agent, such as, for example, bortezomib, thalidomide, dexamethasone, 5-azacitidine, decitabine, vorinostat, or cyclophosphamide. In some embodiments, the anti-cancer agent is a PI3K or mTOR inhibitor, such as rapamycin or a rapamycin analog. In some embodiments, the anti-cancer agent is a gamma secretase inhibitor or an AMPK inducer, such as, for example, metformin or phenformin. In certain embodiments, the anti-cancer agent is an ornithine decarboxylase inhibitor, such as, for example, difluoromethylornithine.

The at least one compound of Formula I or Formula II or tautomer, stereoisomer, pharmaceutically acceptable salt or hydrate thereof may also be administered to treat or prevent and autoimmune and inflammatory diseases or conditions. In other embodiments, at least one compound of Formula I or Formula II, or tautomer, stereoisomer, pharmaceutically acceptable salt or hydrate thereof may also be administered to treat or prevent a disease or disorder resulting from an infection by bacteria or virus, such as for example, HIV, HPV, or herpes. In some embodiments, the disease or disorder to be treated by the methods of the invention is AIDS. In other embodiments, the at least one compound of Formula I or Formula II, or tautomer, stereoisomer, pharmaceutically acceptable salt or hydrate thereof is administered to treat or prevent sepsis in a mammal.

### Preparation of Compounds of Formula I and Formula II

Compounds of Formula I and Formula II and tautomers, stereoisomers, pharmaceutically acceptable salts, and hydrates thereof, may be prepared by any method known in the art. For example, the compounds may be prepared as described in U.S. Published Patent Application 2008/0188467 (see particularly pages 20-22), incorporated herein by reference.

### Example 1

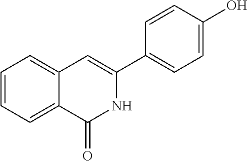 [SMILES: O=c1[nH]c(-c2ccc(O)cc2)cc2ccccc12]

### 3-(4-Hydroxyphenyl)-2H-isoquinolin-1-one

To a solution of n-methyl-o-toluamide (2.0 g, 13.4 mmol) in THF (30 mL), n-butyl lithium (12.3 mL, 30.8 mmol, 2.5 M solution in hexane) was added slowly under nitrogen with cooling (ice-salt bath), maintaining the temperature below 20° C. After completion of addition, the mixture was stirred for 1 h at 0° C., then cooled to −50° C. and a solution of 4-methoxy benzonitrile (2.14 g, 16.08 mmol) in THF (5 mL) was added quickly. The cooling bath was removed and the solution was allowed to warm to room temperature. Saturated aqueous NH₄Cl solution was added with cooling and the solid was isolated by filtration to give the methoxy compound (2.2 g, 65%). The methoxy compound (750 mg, 2.98 mmol) was added to a 50 mL flask and pyridinium hydrochloride (10 g) was added. The mixture was heated at 190° C. for 2 h, then cooled to room temperature, diluted with water, neutralized with NaHCO₃, and the solid was isolated by filtration to give 3-(4-hydroxyphenyl)-2H-isoquinolin-1-one (600 mg, 84%). Selected data: MS (ES) m/z: 238.92, 237.89; MP 239-241° C.

### Example 2

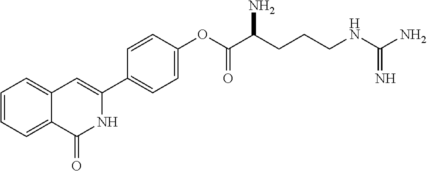 [SMILES: N=C(N)NCCC[C@H](N)C(=O)Oc1ccc(-c2cc3ccccc3c(=O)[nH]2)cc1]

### 4-(1-Oxo-1,2-dihydroisoquinolin-3-yl)phenyl 2-amino-5-guanidinopentanoate trihydrochloride

A mixture of 3-(4-hydroxyphenyl)-2H-isoquinolin-1-one (150 mg, 0.63 mmol) in DMF (5 mL), diisopropyl ethyl amine (245 mg, 1.89 mmol), EDCl (133 mg, 0.696 mmol), Boc-Arg (330 mg, 0.696 mmol) and HOBt (94 mg, 0.696 mmol) was stirred at room temperature for 24 h under nitrogen. The reaction mixture was diluted with water and the solid was collected by filtration. The crude product was purified by column chromatography using 5% MeOH in CH₂Cl₂, to give the tri-Boc ester product (375 mg 85%). HCl gas was bubbled through a solution of the tri-Boc ester (325 mg, 0.468 mmol) in CH₂Cl₂ (10 mL) for 6 h at 0° C. The solid was filtered off and washed with CH₂Cl₂ to give 4-(1-oxo-1,2-dihydroisoquinolin-3-yl)phenyl 2-amino-5-guanidinopentanoate trihydrochloride (170 mg, 72%). Selected data: MS (ES) m/z: 237.25 (M-Arg); ¹³C-NMR (DMSO-d₆): δ 168.8, 163.4, 157.7, 151.0, 139.8, 139.5, 133.4, 132.8, 128.9, 127.4, 127.3, 127.25, 125.6, 122.6, 104.2, 55.6, 52.5, 27.7, 25.0.

### Example 3

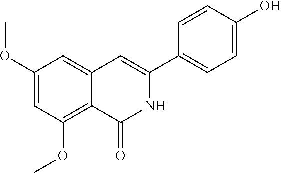 [SMILES: COc1cc(OC)c2c(=O)[nH]c(-c3ccc(O)cc3)cc2c1]

### 3-(4-hydroxyphenyl)-6,8-dimethoxyisoquinolin-1(2H)-one

To a suspension of 2-methyl-4,6-dimethoxy benzoic acid (2.8 g, 14.3 mmol) in CH₂Cl₂ (30 mL), oxalyl chloride (3.62 g, 28.5 mmol) was added and the mixture was stirred at room temperature for 16 h. The solvent and excess oxalyl chloride were removed at reduced pressure. The solid was dissolved in CH₂Cl₂ (10 mL) and methyl amine hydrochloride (1.33 g, 42.81 mmol) was added on cooling and the mixture was stirred at room temperature for 4 h. The solvent was removed and the crude product was purified by chromatography using 5% methanol in CH₂Cl₂, to give 1.3 g of the amide intermediate (43% yield). To a solution of the amide intermediate (1.29 g, 6.16 mmol) in THF (30 mL), n-butyl lithium (5.6 mL, 14.18 mmol, 2.5 M solution in hexane) was added slowly under nitrogen with cooling (ice-salt bath), maintaining the temperature below 20° C. The mixture was stirred for 1 h at 0° C., then cooled to −50° C. and a solution of 4-O-TBDMS-benzonitrile (1.58 g, 6.78 mmol) in THF (10 mL) was added quickly. The cooling bath was removed and the mixture was stirred at room temperature for 16 h. Saturated aqueous NH₄Cl solution was added with cooling, and the layers were separated. The organic layer was washed with water, brine, dried over Na₂SO₄ and concentrated to give the crude intermediate, which was purified by chromatography using 5% methanol in CH₂Cl₂, to give two products (1) 678 mg of isoquinoline in 26% yield and (2) 780 mg of quinalone product in 27% yield. To a suspension of the above quinalone product (780 mg, 1.65 mmol) in ethanol (20 mL), conc. HCl (2 mL) was added and the mixture was heated at 70° C. for 2 h. The reaction mixture was cooled to room temperature and the solvent was removed and purified by chromatography to give 3-(4-hydroxyphenyl)-6,8-dimethoxyisoquinolin-1(2H)-one (215 mg, 44%). Selected data: MS (ES) m/z: 297.93; MP 245-247° C.

### Example 4

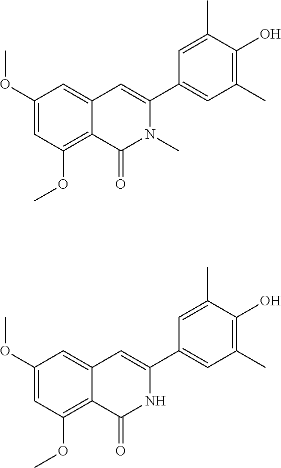 [SMILES: COc1cc(OC)c2c(=O)n(C)c(-c3cc(C)c(O)c(C)c3)cc2c1 ; COc1cc(OC)c2c(=O)[nH]c(-c3cc(C)c(O)c(C)c3)cc2c1]

### 3-(4-hydroxy-3,5-dimethylphenyl)-6,8-dimethoxy-2-methylisoquinolin-1(2H)-one (left) and 3-(4-hydroxy-3,5-dimethylphenyl)-6,8-dimethoxyisoquinolin-1(2H)-one (right)

To a suspension of 2-methyl-4,6-dimethoxy benzoic acid (2.61 g, 13.1 mmol) in CH₂Cl₂ (50 mL), oxalyl chloride (3.38 g, 26.6 mmol) was added and the mixture was stirred at room temperature for 16 h. The solvent and excess oxalyl chloride were removed at reduced pressure. The solid was dissolved in CH₂Cl₂ (10 mL) and methyl amine (1.24 g, 39.9 mmol) with cooling and was stirred at room temperature for 4 h. The solvent was removed and crude product was purified by chromatography by using 5% methanol in CH₂Cl₂ to give the amide (2.27 g, 82%). To a solution of the above amide (2.27 g, 10.9 mmol) in THF (50 mL), n-butyl lithium (9.98 mL, 25.0 mmol, 2.5 M solution in hexane) was added slowly under nitrogen with cooling, maintaining the temperature below 20° C. The mixture was stirred for 1 h at 0° C., then cooled to −50° C., and a solution of 4-O-TBDMS-3,5-dimethyl benzonitrile (2.97 g, 11.39 mmol) in THF (10 mL) was added quickly, the cooling bath was removed and the mixture was stirred for 16 h at room temperature. A saturated aqueous NH₄Cl solution was added with cooling, and the layers were separated. The organic layer was washed with water, brine, dried over Na₂SO₄ and concentrated to give 3.9 g of the crude product mixture. A suspension of the crude product mixture (3.9 g) in ethanol (20 mL) was heated with conc. HCl (2 mL) at 80° C. for 2 h. The reaction mixture was cooled to room temperature and the solvent was removed. The solid was dissolved in water and neutralized by NaHCO₃, followed by extraction with CH₂Cl₂. The product was purified by chromatography to give two products: 3-(4-hydroxy-3,5-dimethylphenyl)-6,8-dimethoxy-2-methylisoquinolin-1(2H)-one (128 mg, 5%) and 3-(4-hydroxy-3,5-dimethylphenyl)-6,8-dimethoxyisoquinolin-1(2H)-one (340 mg, 9%). Selected data for 3-(4-hydroxy-3,5-dimethylphenyl)-6,8-dimethoxy-2-methylisoquinolin-1(2H)-one: MS (ES) m/z: 340.01 (M); MP 253-254° C. Selected data for 3-(4-hydroxy-3,5-dimethylphenyl)-6,8-dimethoxyisoquinolin-1(2H)-one: MS (ES) m/z: 326.00; MP 226-227° C.

### Example 5

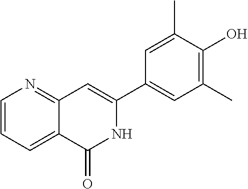 [SMILES: Cc1cc(-c2cc3ncccc3c(=O)[nH]2)cc(C)c1O]

### 7-(4-hydroxy-3,5-dimethylphenyl)-1,6-naphthyridin-5(6H)-one

I chloride (1.90 mL, 21.8 mmol) was added to 2-methyl nicotinic acid (1.50 g, 10.9 mmol) in anhydrous dichloromethane (20 mL) with triethylamine (1.6 mL, 11.5 mmol) and the reaction mixture was kept at room temperature overnight before the solvent was removed. THF was added to the residue and ammonia gas was bubbled through for 2 h. The THF was removed and the residue was dissolved into methanol and water and the pH was adjusted to 10.0 with potassium carbonate. The mixture was concentrated. After column chromatography the desired amide was isolated (1.10 g, 73.8%).

NaH (0.428 g, 10.7 mmol, 60% in mineral oil) was added to 4-hydroxy-3,5-dimethylbenzonitrile (1.50 g, 10 mmol) in anhydrous DMF (8 mL). Benzyl bromide (1.83 g, 10.7 mmol) was added and the reaction was kept at room temperature overnight. The reaction mixture was poured into water. The isolated solid was further washed with hexane to yield the desired ether building block (2.0 g, 84.3%). It was used in the next reaction without further purification. The above amide (0.65 g, 4.77 mmol) in anhydrous THF (15 mL) was added drop-wise to BuLi (7.5 mL, 1.60 M) at −20° C. The reaction mixture was kept at this temperature for 1 h and then the above ether building block (1.13 g, 4.77 mmol) in THF (20 mL) was added drop-wise at −20° C. and the reaction was stirred for 1.5 h. The reaction temperature was increased to room temperature and continued for a further 1 h. Water (20 mL) was added and the mixture was stirred for a while before the solvent was removed and the residue was purified by column chromatography to yield the desired intermediate (0.50 g, 29.4%). A 50 mL flask was charged with the above described intermediate (0.50 g, 0.0014 mol) and pyridine hydrogen chloride (2.4 g, 0.014 mol) and the mixture was heated to 180° C. for 1.5 h. The mixture was cooled and poured into methanol (4 mL), then filtered. The collected solid was further washed with ethyl acetate and dried to give 7-(4-hydroxy-3,5-dimethylphenyl)-1,6-naphthyridin-5(6H)-one (350 mg, 82.7%) as an HCl salt. Selected data: MS (ES) m/z: 266; MP >350° C.

### Example 6

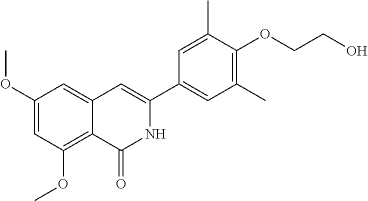 [SMILES: COc1cc(OC)c2c(=O)[nH]c(-c3cc(C)c(OCCO)c(C)c3)cc2c1]

### 3-(4-(2-hydroxyethoxy)-3,5-dimethylphenyl)-6,8-dimethoxyisoquinolin-1(2H)-one

To a solution of 3,5-dimethyl-4-hydroxy benzonitrile (1.0 g, 6.79 mmol) in DMF (100 mL), were added a NaH (1.065 g, 26.63 mmol) and (2-bromoethoxy)-tert-butyl dimethyl silane (1.95 g, 8.15 mmol). The reaction mixture was stirred for 10 d at room temperature under nitrogen. The reaction mixture was poured into ice-water and the products were extracted with ethyl acetate. The organic layer was separated, washed with water, dried and concentrated to give crude product, which was purified by column chromatography to give 1.9 g of the B-ring building block in 92% yield.

n-Butyl lithium (2.84 mL, 7.1 mmol, 2.5 M solution in hexane) was added slowly to a solution of 2,4-dimethoxy-6-methyl benzamide (650 mg, 3.1 mmol) in THF (30 mL), under nitrogen with cooling (ice-salt bath), maintaining the temperature below 20° C. After completion of addition, the mixture was stirred for 1 h at 0° C., and then cooled to −50° C. and a solution of 4-(2-tert-butyldimethyl silanyloxy)ethoxy)-3,5-dimethyl benzonitrile (the B-ring building block, above) (996 mg, 3.26 mmol) in THF (10 mL) was added quickly. The cooling bath was removed and the reaction mixture was allowed to warm to room temperature and was stirred for 16 h at room temperature. A saturated NH₄Cl solution was added with cooling, and the layers were separated. The organic layer was washed with water, brine, dried over Na₂SO₄ and concentrated to give 1.2 g of crude product.

The above crude product (1.2 g) was treated with ethanol (10 mL) and conc. HCl (2 mL) at 80° C. for 1 h. The solvent was removed and the residue was dissolved in methanol and neutralized by NaHCO₃. The solvent was evaporated and crude product was purified by column chromatography to give 3-(4-(2-hydroxyethoxy)-3,5-dimethylphenyl)-6,8-dimethoxyisoquinolin-1(2H)-one (100 mg, 11%). Selected data: MP 193-195° C.

### Example 7

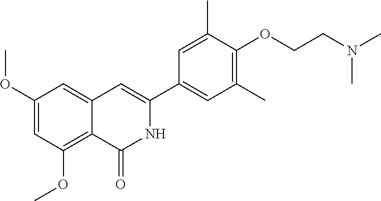 [SMILES: COc1cc(OC)c2c(=O)[nH]c(-c3cc(C)c(OCCN(C)C)c(C)c3)cc2c1]

### 3-(4-(2-(dimethylamino)ethoxy)-3,5-dimethylphenyl)-6,8-dimethoxyisoquinolin-1(2H)-one

In a 250 mL round-bottomed flask were placed 3,5-dimethyl-4-hydroxybenzonitrile (1.0 g, 6.79 mmol), Ph₃P (1.96 g, 7.47 mmol), di-isopropylethylamine (1.75 g, 13.59 mmol) and 2-dimethylaminoethanol (660 mg, 7.47 mmol) in THF (30 mL). DEAD (1.42 g, 8.15 mmol) was added drop-wise at room temperature. The reaction mixture was stirred for 48 h at room temperature and water was added and the mixture was extracted with ethyl acetate. The combined organic layers were washed with water, brine and dried over Na₂SO₄ and concentrated to give crude product. The crude product was purified by column chromatography to give 1.17 g (79%) of the B-ring building block.

n-Butyl lithium (4.2 mL, 10.54 mmol, 2.5 M solution in hexane) was added slowly to a solution of 2,4-dimethoxy-6-methyl benzamide (958 mg, 4.58 mmol) in THF (30 mL) under nitrogen with cooling (ice-salt bath), maintaining the temperature below 20° C. After completion of the addition, the mixture was stirred for 1 h at 0° C., then cooled to −50° C. and a solution of 4-(2-dimethylamino ethoxy)-3,5-dimethyl benzonitrile (1.1 g, 5.04 mmol) (the B-ring building block) in THF (10 mL) was added quickly. The cooling bath was removed and the reaction mixture was allowed to warm to room temperature and stirred for 16 h at room temperature. A saturated NH₄Cl solution was added with cooling and the layers were separated. The organic layer was washed with water, brine, dried over Na₂SO₄ and concentrated to give crude product. The crude product was purified by chromatography to give 3-(4-(2-(dimethylamino) ethoxy)-3,5-dimethylphenyl)-6,8-dimethoxyisoquinolin-1(2H)-one (162 mg, 8%) as a hydrochloride. Selected data: MS (ES) m/z: 397.06; MP 261-263° C. at decomposition (HCl).

### Example 8

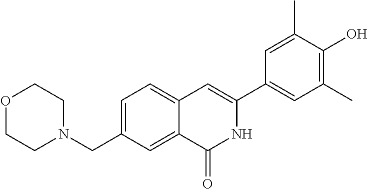 [SMILES: Cc1cc(-c2cc3ccc(CN4CCOCC4)cc3c(=O)[nH]2)cc(C)c1O]

### 3-(4-hydroxy-3,5-dimethylphenyl)-7-(morpholinomethyl)isoquinolin-1(2H)-one

Hydrogen bromide in acetic acid (13 mL, 33 wt %) was added to a mixture of 2-methyl benzoic acid (4.08 g, 30 mmol), paraformaldehyde (2.50 g, 83.0 mmol), and o-phosphoric acid (7 mL, 85%). The reaction mixture was stirred at 115° C. for 15 h. It was cooled to room temperature and poured into ice-cold water. A white precipitate was formed. The mixture was extracted with ethyl acetate (300 mL). The organic layer was washed with water (100 mL), brine (100 mL) and dried over anhydrous Na₂SO₄. Removal of solvent gave 6.84 g of a white solid, which was used in the next step without further purification. The above compound (6.8 g) was dissolved in anhydrous dichloromethane (150 mL). Oxalyl chloride (7.8 mL) was added drop-wise. After the addition was complete, 3 drops of anhydrous DMF were added. A vigorous reaction occurred and the stirring was continued overnight. Solvent and excess oxalylchloride were removed under reduced pressure and the residue was dried under vacuum to give 7.02 g of brown liquid, which was used in the next step without further purification. The above compound (7.02 g, 28.36 mmol) was dissolved in anhydrous THF (60 mL) and cooled to 0° C. A solution of N-methylamine (2.0 M in THF, 19 mL, 38.03 mmol) was added drop-wise under nitrogen. The stirring was continued for 15 min at 0° C. The ice-bath was removed, and the stirring was continued at room temperature for 3 h. A white precipitate was formed. Water (100 mL) was added and the mixture was extracted with ethyl acetate (150 mL). The organic layer was separated, washed with water (50 mL), saturated NaHCO₃ solution (2×50 mL), water (50 mL), and brine (50 mL), and dried over anhydrous Na₂SO₄. Removal of solvent gave 5.64 g of 5-bromomethyl-2,N-dimethylbenzamide as a white solid which was used in the next step without further purification. To a solution of the above compound (2.42 g, 10 mmol) in anhydrous THF was added morpholine (1.92 g, 22 mmol) at room temperature under nitrogen. A white precipitate was formed. Stirring continued overnight. Water (100 mL) was added and the mixture was extracted with ethyl acetate (150 mL). The organic layer was separated, washed with water (50 mL) and brine (50 mL) and dried (Na₂SO₄). Removal of solvent gave a colorless oil, which was purified by column chromatography (silica gel 230-400 mesh; 0-5% methanol in CH₂Cl₂ as eluent) to give the desired benzamide intermediate (yield 0.50 g, 20%). N-Butyl lithium (1.6 M solution in hexanes, 4.1 mL, 6.6 mmol) was added drop-wise to a solution of the benzamide (0.5 g, 2.0 mmol) in anhydrous THF (4 mL) at −10° C. over a period of 10 min under nitrogen. Stirring was continued at 0° C. for 1 h. The reaction mixture was cooled to −50° C. A solution of 4-(tert-butyldimethylsilanyloxy)-3,5-dimethylbenzonitrile (0.653 g, 2.5 mmol) in anhydrous THF (3 mL) was quickly added. The cooling bath was removed and the reaction mixture was allowed to warm to room temperature. Stirring was continued at room temperature for 1 h. An aqueous ammonium chloride solution (5 mL) was added followed by ethyl acetate (50 mL). The organic layer was separated, washed with water (5 mL) and dried (Na₂SO₄). Removal of the solvent gave 1.23 g pale yellow gummy material, which was used in next step without further purification. The above compound (1.2 g) was dissolved in 10 mL anhydrous ethanol. Conc. HCl (1 mL) was added and the mixture was refluxed for 15 min, then cooled to room temperature. The solvent was removed under reduced pressure. The crude compound was basified with methanolic ammonia and purified by column chromatography (silica gel 230-400 mesh; 0-5% methanol in CH₂Cl₂ as eluent) to give 3-(4-hydroxy-3,5-dimethylphenyl)-7-morpholin-4-ylmethyl-2H-isoquinolin-1-one (35 mg) as a white solid (the free base). To a solution of the above compound (35 mg) in CH₂Cl₂ (5 mL) and MeOH (1 mL) was added drop-wise hydrogen chloride in ether (0.5 mL, 1.0 M) under nitrogen. The reaction mixture was stirred at room temperature for 1 h. The solvent was removed under reduced pressure and dried under vacuum to give the hydrochloride of 3-(4-hydroxy-3,5-dimethylphenyl)-7-(morpholinomethyl)isoquinolin-1(2H)-one (36 mg, 93%) as a yellow solid. Selected data: MP 281-283° C. (hydrochloride).

### Example 9

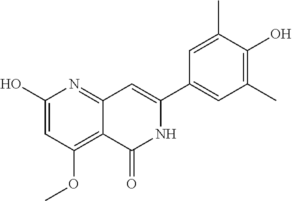 [SMILES: COc1cc(O)nc2cc(-c3cc(C)c(O)c(C)c3)[nH]c(=O)c12]

### 2-hydroxy-7-(4-hydroxy-3,5-dimethylphenyl)-4-methoxy-1,6-naphthyridin-5(6H)-one

A mixture of malonic acid (20 g, 192 mmol), 2,4,6-trichlorophenol (72 g, 365 mmol), and phosphorus oxychloride (38 mL, 403.2 mmol) was stirred at reflux for 12 h. The reaction mixture was cooled to 70° C. and poured into ice water. The solid was collected by filtration, washed with water, and air-dried to give malonic acid bis-(2,4,6-trichloro-phenyl) ester (85 g, 95%). A solution of malonic acid bis-(2,4,6-trichloro-phenyl) ester (85 g, 183.6 mmol) and ethyl 3-aminocrotonate (26.1 g, 202 mmol) in bromobenzene (100 mL) was stirred at reflux for 50 min. The reaction mixture was cooled to 50° C. and diluted with EtOAc (260 mL). The solid was collected by filtration, washed with water, and air-dried, to give 4,6-dihydroxy-2-methyl nicotinic acid ethyl ester (31 g, 86%).

A solution of 4,6-dihydroxy-2-methyl nicotinic acid ethyl ester (31 g, 157 mmol) in phosphorus oxychloride (60 mL, 629 mmol) was stirred at reflux for 1.5 h. The extra phosphorus oxychloride was removed and the reaction mixture was poured into ice water. The solid was removed by filtration. The filtrate was extracted with dichloromethane (3×100 mL) and concentrated. The residue was further purified by column chromatography to yield 4,6-dichloro-2-methyl nicotinic acid ethyl ester (16.9 g, 46%). A solution the ester (16.9 g, 71.3 mmol) in MeOH (60 mL) was mixed with sodium methoxide (58 mL, 257 mmol) and stirred at reflux for 12 h. The reaction was quenched by adding AcOH (50 mL), diluted with water (200 mL), extracted with dichloromethane (3×100 mL), and concentrated. The residue was purified by column chromatography to yield 4,6-dimethoxy-2-methyl nicotinic acid methyl ester (10 g, 67%). A solution of the ester (2.6 g, 12.3 mmol), lithium hydroxide (1.06 g, 44.1 mmol) in water (40 mL), MeOH (30 mL) and THF (20 mL) was stirred at reflux for 4 h. The reaction mixture was concentrated to dryness. The residue was mixed with HCl (conc., 20 mL) and was concentrated to dryness to yield crude 4,6-dimethoxy-2-methyl nicotinic acid (quantitative). To a solution of 4,6-dimethoxy-2-methyl nicotinic acid (2.5 g, 12.0 mmol) in dichloromethane (50 mL) and THF (50 mL) at room temperature was added oxalyl chloride (2.57 mL, 29.4 mmol) and DMF (3 drops). The reaction mixture was stirred at room temperature for 0.5 h, concentrated to afford 4,6-dimethoxy-2-methyl nicotinic acid chloride HCl salt (2.8 g). A solution of 4,6-dimethoxy-2-methyl nicotinic acid chloride HCl salt (8.5 g, 33.73 mmol) in dichloromethane (20 mL) and THF (20 mL) at room temperature was mixed with methylamine in THF (50 mL, 98 mmol) and stirred at 20° C. for 1 h. The reaction mixture was diluted with water (100 mL), extracted with dichloromethane (3×100 mL), and concentrated to yield 4,6-dimethoxy-2,N-dimethyl-nicotinamide (4.2 g, 66%) as a light yellow solid. A solution of 4-hydroxy-3,5-dimethylbenzonitrile (2 g, 13.6 mmol) in DMF (20 mL) at room temperature was mixed with sodium hydride (0.706 g, 17.6 mmol) and stirred for 0.5 h. Benzyl bromide (1.62 mL, 13.59 mmol) was then added and the reaction mixture was stirred at room temperature for 24 h. The reaction was quenched by adding water (200 mL), extracted with EtOAc (3×100 mL), and concentrated. The residue was purified by column chromatography to yield 4-benzyloxy-3,5-dimethylbenzonitrile (3.25 g, 100%), as a white solid. To a solution of 4,6-dimethoxy-2,N-dimethyl-nicotinamide (0.54 g, 2.57 mmol) in THF (50 mL) at −20° C. was added n-BuLi (3.54 mL, 5.67 mmol). The reaction was stirred at −20° C. to 0° C. for 2 h and then was cooled to −78° C. 4-Benzyloxy-3,5-dimethylbenzonitrile (0.49 g, 2.057 mmol) was added, the cooling bath was removed, and the reaction was allowed to warm to room temperature. After 14 h, the reaction was quenched by adding water (100 mL), extracted with dichloromethane (3×100 mL), and concentrated. The residue was purified by column chromatography to yield 7-(4-benzyloxy-3,5-dimethyl-phenyl)-2,4-dimethoxy-6H-[1,6]naphthyridin-5-one (0.32 g, 37%). A solution of 7-(4-benzyloxy-3,5-dimethyl-phenyl)-2,4-dimethoxy-6H-[1,6]naphthyridin-5-one (0.25 g, 0.6 mmol) in dichloromethane (100 mL) was mixed with BBr₃ (3 mL, 3 mmol) and stirred at room temperature for 16 h. The reaction was quenched by adding water (20 mL). The resulting solid was collected by filtration, washed with water and DCM, to yield a light yellow solid. This solid was mixed with HCl in ether (10 mL, 10 mmol), stirred for 1 h, and filtered to afford 2-hydroxy-7-(4-hydroxy-3,5-dimethylphenyl)-4-methoxy-1,6-naphthyridin-5(6H)-one hydrochloride (70 mg, 37%) as a light yellow solid. Selected data: MS (ES) m/z: 312; MP >330° C. (hydrochloride).

### Example 10

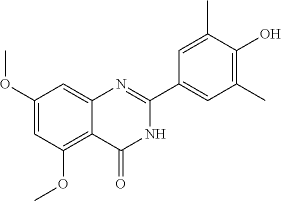 [SMILES: COc1cc(OC)c2c(=O)[nH]c(-c3cc(C)c(O)c(C)c3)nc2c1]

### 2-(4-hydroxy-3,5-dimethylphenyl)-5,7-dimethoxyquinazolin-4(3H)-one

A solution of 3,5-dimethoxyaniline (199 g, 1.30 mol) in ether (5.0 L) in a 5 L 3-necked flask was cooled to 0° C. HCl gas (227 g) was bubbled through the solution over 45 min. After 45 min at 10° C., the mixture was filtered, washed with isopropylacetate (4 L), and dried overnight on high vacuum at 45° C. to give the hydrochloride (242.3 g, 98%), as a white solid. A mixture of the hydrochloride above (20 g, 0.105 mol) and oxalyl chloride (33 mL) in a 3-necked flask equipped with a reflux condenser was heated for 2 h with stirring (170° C. external temperature), and the oxalyl chloride was distilled from the reaction mixture. The flask was cooled to 0° C. and methanol (40 mL) was added. The reaction mixture was heated to reflux for 45 min, filtered while hot, and washed with methanol (80 mL) to give the 4,6-dimethoxyisatin (17.2 g, 79%) as a yellow-green solid. To a heated solution (external temp 70° C.) of the isatin (162 g, 0.78 mol) in aqueous NaOH (40%, 1.5 L) was added H₂O₂ (35%, 405 mL) slowly over 2 h. After the addition of each portion of H₂O₂, the internal reaction temperature (initially 64° C.) increased (to a maximum temp of 80° C.). After the addition was complete, the foaming reaction mixture was then stirred for an additional 2 h at 70° C., and the mixture was allowed to stir overnight while cooling to room temperature. The mixture was heated to 70° C. Additional H₂O₂ (75 mL) was added, and the mixture was stirred at 70° C. for a further 2 h until the reaction was complete. After cooling to 10° C. (bath temperature), aqueous Na₂S₂O₃ (150 mL, saturated) was added. The mixture was brought to pH 8 with HCl (37%, 1.6 L) and pH 6 with acetic acid (glacial, 75 mL), without allowing the reaction mixture to warm to greater than 40° C. Filtration of the reaction mixture and washing with water (4 L) gave the expected amino acid as a tan solid (83.7 g, 55%). To a solution of the amino acid (82.7 g, 0.42 mol) in anhydrous THF (4.2 L) was added EDCl (89.2 g, 0.48 mol), HOBT (65 g, 0.48 mol), and NMM (51.3 mL), and the mixture was allowed to stir at room temperature for 3 h. Aqueous NH₃ (83 mL, 50%) was added, and the mixture was stirred at room temperature for 16 h. Water (1.25 L) was added, and the mixture was extracted with DCM (2×250 mL). The combined extracts were then washed with water (2×500 mL). Concentration, formation of a slurry with ether (550 mL), filtration, and drying under high vacuum gave 2-amino-4,6-dimethoxybenzamide (46.7 g, 57%) as a brown solid.

2-Amino-4,6-dimethoxy-benzamide (1.06 g, 5.4 mmol), 3,5-dimethyl-4-hydroxybenzaldehyde (0.810 g, 5.4 mmol), K₂CO₃ (0.747 g, 5.4 mmol) and I₂ (1.645 g, 6.5 mmol) were mixed in DMF (20 mL) and the reaction mixture was heated at 80° C. for 12 h. It was cooled to room temperature and poured into crushed ice. The solid was collected and purified by column chromatography to give 2-(4-hydroxy-3,5-dimethylphenyl)-5,7-dimethoxyquinazolin-4(3H)-one (0.9 g, 51%) as a white solid. Selected data: MP 291-293° C.

### Example 11

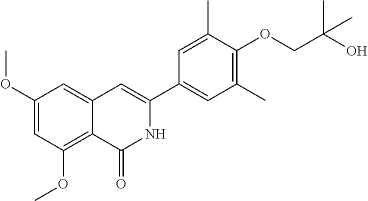 [SMILES: COc1cc(OC)c2c(=O)[nH]c(-c3cc(C)c(OCC(C)(C)O)c(C)c3)cc2c1]

### 3-(4-(2-hydroxy-2-methylpropoxy)-3,5-dimethylphenyl)-6,8-dimethoxyisoquinolin-1(2H)-one

To a solution of 4-hydroxy-3,5-dimethylbenzonitrile (2.00 g, 13.5 mmol) and 1-chloro-2-methyl propan-2-ol (8.85 g, 81.5 mmol) in ethanol (50 mL) was added potassium carbonate (7.5 g, 54 mmol) and water (5 mL). The reaction mixture was stirred at reflux for 24 h and cooled to room temperature. The precipitated solid was filtered off and washed with water. The solid was dissolved in ethyl acetate (100 mL), washed with water (50 mL), brine (50 mL), and dried over anhydrous Na₂SO₄. Removal of solvent gave 4-(2-hydroxy-2-methylpropoxy)-3,5-dimethyl benzonitrile (2.9 g, 97%) as a white solid.

To a solution of 4-(2-hydroxy-2-methylpropoxy)-3,5-dimethyl benzonitrile (2.90 g, 13.2 mmol) in anhydrous DMF (20 mL) was added imidazole (2.7 g, 40 mmol) and tert-butyldimethylsilylchloride (2.19 g, 14.6 mmol). The reaction mixture was stirred at room temperature under nitrogen for 3 d. Water (200 mL) was added and the mixture was extracted with ethyl acetate (200 mL). The organic layer was washed with water (2×100 mL) and brine (100 mL), and dried over anhydrous Na₂SO₄. The solvent was removed under reduced pressure and the crude compound was purified by column chromatography to give 4-[2-(tert-butyldimethylsilanyloxy)-2-methylpropoxy]-3,5-dimethylbenzonitrile (2.24 g, 54%). N-Butyl lithium (6.2 mL, 6.6 mmol, 1.6 M solution in hexanes) was added to a solution of 2,4-dimethoxy-6-N-dimethylbenzamide (0.9 g, 4.3 mmol) in anhydrous THF (10 mL) drop-wise at −10° C. over a period of 10 min under nitrogen. The stirring was continued at 0° C. for 1 h. The reaction mixture was cooled to −50° C. A solution of 4-[2-(tert-butyldimethylsilanyloxy)-2-methylpropoxy]-3,5-dimethylbenzonitrile (1.58 g, 4.73 mmol) in anhydrous THF (5 mL) was quickly added. The cooling bath was removed and the reaction mixture was allowed to warm to room temperature. The stirring was continued at room temperature for 1 h. An aqueous ammonium chloride solution (10 mL) was added followed by ethyl acetate (100 mL). The organic layer was separated, washed with water (10 mL) and dried (Na₂SO₄). The solvent was removed under reduced pressure and the crude compound was purified by column chromatography (silica gel 230-400 mesh; 0-5% methanol in CH₂Cl₂ as eluent) to give 3-{4-[2-(tert-butyldimethylsilanyloxy)-2-methylpropoxy]-3,5-dimethylphenyl}-6,8-dimethoxy-2H-isoquinolin-1-one (0.82 g, 37%), as a white solid.

The above compound (0.42 g, 0.82 mmol) was dissolved in anhydrous THF (20 mL). Tetrabutylammonium fluoride (4.1 mL, 1.0 M solution in THF) was added at 0° C. The reaction mixture was stirred at 0° C. for 10 min, then at room temperature for 2 h and then stirred at 70° C. for 24 h. The mixture was cooled to room temperature. Saturated aqueous ammonium chloride (30 mL) was added. The organic layer was separated, washed with water, brine, and dried over anhydrous Na₂SO₄. The solvent was removed under reduced pressure. The crude product was purified by column chromatography (silica gel 230-400 mesh; 0-4% methanol in CH₂Cl₂ as eluent) to give 3-(4-(2-hydroxy-2-methylpropoxy)-3,5-dimethylphenyl)-6,8-dimethoxyisoquinolin-1(2H)-one (0.15-g, 46%), as a white solid. Selected data: MS (ES) m/z: 397.98; MP 252-254° C. at decomposition.

### Example 12

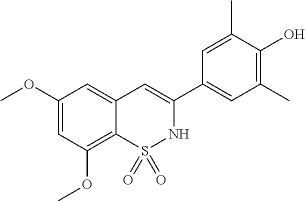

### 6,8-dimethoxy-3-(4-hydroxy-3,5-dimethylphenyl)-2H-1,2-benzothiazine-1,1-dioxide

To a 3-necked, round-bottomed flask was added 3,5-dimethoxytoluene (6.088 g, 40 mmol) and cyclohexane (28 mL) under nitrogen. Dimethyl carbonate (30.3 g, 336 mmol) was added and the reaction mixture was heated at 60° C. Excess chlorosulfonic acid was added over a period of 15 min. The liberated HCl gas was removed by inserting a tube into solid sodium hydroxide. On completion of the addition, the reaction mixture was heated to 70-72° C. for 1 h and then cooled to room temperature. The solid was filtered off and washed with dimethyl carbonate/cyclohexane (1:1, 20 mL). The solid was dried in vacuo to obtain pure material (6.13 g, 66%). To a mixture of the sulfonic acid (product from above, 4.65 g, 20 mmol) and triethyl amine (2.03 g, 2.79 mL) in acetone (40 mL) was added 2,4,6-trichloro-1,3,5-triazine (cyanuric chloride, 3.69 g, 20 mmol). The reaction mixture was heated under reflux for 20 h before being cooled to room temperature. The solution was passed through a Celite pad and evaporated in vacuo to leave a solid, which was filtered off and washed with hexane. The mixture of product and salt of cyanuric hydroxide and triethyl amine (7.58 g) was used for the next step without further purification.

To a 3-necked, round-bottomed flask, equipped with a condenser (acetone-dry ice cooling), was added the mixture from the step above (7.58 g) and acetone (100 mL). The reaction mixture was cooled to −78° C. and ammonia gas was bubbled through the solution for 0.5 h. The reaction mixture was kept standing overnight, allowing slow evaporation of ammonia gas, followed by the evaporation of solvent. Water was added and the product was extracted with DCM. The solvent was dried and evaporated to leave a mixture of solid and a dense liquid. The solid was filtered off and washed with hexane to leave pure sulfonamide (3.23 g, 70%).

To a round-bottomed flask was added 3,5-dimethyl-4-hydroxybenzoic acid (2.99 g, 18 mmol). Anhydrous DMF (20 mL) was added, followed by sodium hydride (1.8 g, 45 mmol). The reaction mixture was stirred at room temperature for 1 h. p-Methoxybenzyl chloride (6.20 g, 39.6 mmol) was added and the mixture was stirred at room temperature overnight (˜20 h). The reaction mixture was poured into water, acidified with 1 N HCl and stirred for 1 h. The precipitated solid was filtered off, washed with water and hexane to obtain pure B-ring building block (6.93 g, 95%).

The B-ring building block (6.93 g, 17.1 mmol) was dissolved in a mixture of methanol (50 mL) and tetrahydrofuran (50 mL). Potassium hydroxide (1.25 g, 22.2 mmol) in water (20 mL) was added. The reaction mixture was refluxed at 70° C. for 24 h. The solvent was evaporated in vacuo. Water was added and the reaction mixture was acidified with 1 N HCl (pH 4-5). The solid was filtered off, washed with water and hexane. The yield was 4.61 g (94%). The product (1.932 g, 6.75 mmol) and the sulfonamide from above (1.04 g, 4.5 mmol) were taken in a 3-necked, round-bottomed flask under nitrogen. Dichloromethane (100 mL) was added with stirring. To this stirred mixture was added N-(3-dimethylaminopropyl)-N′-ethylcarbodiimide hydrochloride (EDCl. HCl, 1.36 g, 7.09 mmol), followed by N,N-dimethylaminopyridine (2.06 g, 16.9 mmol). The reaction mixture was stirred at room temperature for 24 h before being washed with 1 N HCl, 2.5% NaOH and saturated sodium bicarbonate solutions. The organic layers were dried and evaporated in vacuo to leave a residue, which was purified by silica gel (100 g) column chromatography, employing 20-50% ethyl acetate in hexane and 5% methanol in dichloromethane as eluents. Fractions 30-66 were combined to obtain pure materials (1.35 g, 60%). The compound from the step above (0.105 g, 0.21 mmol) was dissolved in tetrahydrofuran under nitrogen and cooled to −78° C. n-Butyllithium was added and the reaction mixture was allowed to warm to room temperature slowly and stirred overnight (˜14 h). TLC showed incomplete conversion. The reaction mixture was quenched with saturated ammonium chloride solution and extracted with ethyl acetate. The solvent was evaporated in vacuo to leave a residue that was purified by silica gel (15 g) column chromatography, employing 20-50% ethyl acetate in hexane as eluents. The product was not pure enough, so another column was used, employing 0.5% methanol in hexane as eluent, and finally preparative TLC was employed to purify the material. The compound from the step above (0.277 g) was dissolved in trifluoroacetic acid (10 mL) under nitrogen and the reaction mixture was refluxed (bath temperature 80° C.) for 4 d. The solvent was evaporated in vacuo and the residue was dissolved in 0.25 N NaOH (20 mL), and acidified with acetic acid. The solid had precipitated out at this point. The solid was filtered off and washed with water, hexane and dried. From one batch, 0.005 g of pure material was isolated. From another batch, 0.060 g compound was isolated, which was not pure enough. This compound was further purified by preparative HPLC to give pure 6,8-dimethoxy-3-(4-hydroxy-3,5-dimethylphenyl)-2H-1,2-benzothiazine-1,1-dioxide (0.010 g). Selected data: MP 246.6-247.4° C.

### Example 13

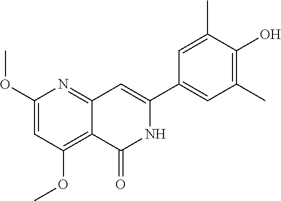 [SMILES: COc1cc(OC)c2c(=O)[nH]c(-c3cc(C)c(O)c(C)c3)cc2n1]

### 7-(4-hydroxy-3,5-dimethylphenyl)-2,4-dimethoxy-1,6-naphthyridin-5(6H)-one

A mixture of malonic acid (20 g, 192 mmol), 2,4,6-trichlorophenol (72 g, 365 mmol), and phosphorus oxychloride (38 mL, 403.2 mmol) was stirred at reflux for 12 h. The reaction mixture was cooled to 70° C. and poured into ice water. The solid was collected by filtration, washed with water, and dried to give malonic acid bis-(2,4,6-trichloro-phenyl) ester (85 g, 95%). A solution of malonic acid bis-(2,4,6-trichloro-phenyl) ester (85 g, 184 mmol) and ethyl 3-aminocrotonate (26.08 g, 201.9 mmol) in bromobenzene (100 mL) was stirred at reflux for 50 min. The reaction mixture was cooled to 50° C. and diluted with EtOAc (260 mL). The solid was collected by filtration, washed with water, and dried to give 4,6-dihydroxy-2-methyl nicotinic acid ethyl ester (31 g, 86%). A solution of 4,6-dihydroxy-2-methyl nicotinic acid ethyl ester (31 g, 157 mmol) in phosphorus oxychloride (60 mL, 629 mmol) was stirred at reflux for 1.5 h. The extra phosphorus oxychloride was removed and the reaction mixture was poured into ice water. The solid was removed by filtration. The filtrate was extracted with dichloromethane (3×100 mL) and concentrated. The residue was further purified by column chromatography, to yield 4,6-dichloro-2-methyl nicotinic acid ethyl ester (16.9 g, 46%). A solution of 4,6-dichloro-2-methyl nicotinic acid ethyl ester (16.9 g, 71.3 mmol) in MeOH (60 mL) was mixed with sodium methoxide (58 mL, 256.68 mmol) and stirred at reflux for 12 h. The reaction was quenched by adding HOAc (50 mL). The mixture was diluted with water (200 mL), extracted with dichloromethane (3×100 mL), and concentrated. The residue was purified by column chromatography (SiO₂, hexanes/EtOAc=6:1), to yield 4,6-dimethoxy-2-methyl nicotinic acid methyl ester (10 g, 67%). A solution of 4,6-dimethoxy-2-methyl nicotinic acid methyl ester (2.6 g, 12.3 mmol), lithium hydroxide (1.06 g, 44.08 mmol) in water (40 mL), MeOH (30 mL) and THF (20 mL) was stirred at reflux for 4 h. The reaction mixture was concentrated to dryness. The residue was mixed with HCl (conc., 20 mL) and was concentrated again on high vacuum to dryness to yield crude 4,6-dimethoxy-2-methyl nicotinic acid (quantitative yield). To a solution of 4,6-dimethoxy-2-methyl nicotinic acid (2.5 g, 12.0 mmol) in dichloromethane (50 mL) and THF (50 mL) at room temperature was added oxalyl chloride (2.57 mL, 29.4 mmol) and DMF (3 drops). The reaction mixture was stirred at room temperature for 0.5 h, concentrated to dryness using a rotary evaporator to afford crude 4,6-dimethoxy-2-methyl nicotinic acid chloride HCl salt (2.8 g, quantitative). A solution of 4,6-dimethoxy-2-methyl nicotinic acid chloride HCl salt (4.8 g, 23.5 mmol) in dichloromethane (100 mL) at room temperature was poured into a beaker of ammonium hydroxide (200 mL). The reaction mixture was stirred at room temperature for 1 h, extracted with dichloromethane (3×100 mL), and concentrated using a rotary evaporator to yield 4,6-dimethoxy-2-methyl-nicotinamide (2.4 g, 52%) as a light yellow solid. A solution of 4-hydroxy-3,5-dimethylbenzonitrile (2.00 g, 13.59 mmol) in DMF (20 mL) at room temperature was mixed with sodium hydride (0.706 g, 17.6 mmol) and stirred for 0.5 h. Benzyl bromide (1.62 mL, 13.59 mmol) was added and the reaction mixture was stirred at room temperature for 24 h. The reaction was quenched by adding water (200 mL), extracted with EtOAc (3×100 mL), and concentrated. The residue was purified by column chromatography to yield 4-benzyloxy-3,5-dimethylbenzonitrile (3.25 g, 100%) as a white solid. To a solution of 4,6-dimethoxy-2-methyl-nicotinamide (1 g, 5.1 mmol) in THF (120 mL) at −20° C. was added n-BuLi (9.6 mL, 15.3 mmol). The reaction was stirred at −20-0° C. for 2.5 h and then was cooled to −78° C. 4-Benzyloxy-3,5-dimethylbenzonitrile (1.21 g, 5.1 mmol) was added, the cooling bath was removed, and the reaction was allowed to warm up gradually to room temperature. After stirring at room temperature for 20 h the reaction was quenched by adding water (100 mL), extracted with dichloromethane (3×100 mL), and concentrated using a rotary evaporator. The residue was further purified by column (SiO₂, Hexanes/EtOAc/MeOH=3:2:1) to yield 7-(4-benzyloxy-3,5-dimethyl-phenyl)-2,4-dimethoxy-[1,6]naphthyridin-5-ylamine (0.4 g, 19%) and 7-(4-benzyloxy-3,5-dimethyl-phenyl)-2,4-dimethoxy-6H-[1,6]naphthyridin-5-one (0.34 g, 16%). A solution of 7-(4-benzyloxy-3,5-dimethyl-phenyl)-2,4-dimethoxy-6H-[1,6]naphthyridin-5-one (0.34 g, 0.82 mmol) in DMF (100 mL) and MeOH (100 mL) was mixed with palladium/carbon (0.1 g) and subjected to hydrogenation (50 psi) for 2 h. The mixture was filtered through a Celite-pad. The filtrate was concentrated on high vacuum to afford 7-(4-hydroxy-3,5-dimethyl-phenyl)-2,4-dimethoxy-6H-[1,6]naphthyridin-5-one (0.23 g, 88%). A solution of 7-(4-hydroxy-3,5-dimethyl-phenyl)-2,4-dimethoxy-6H-[1,6]naphthyridin-5-one (0.23 g, 0.7 mmol) in MeOH (20 mL) and DCM (20 mL) was mixed with HCl in ether (7 mL, 7 mmol) and stirred for 0.5 h. The reaction was concentrated using a rotary evaporator to get a solid residue. The solid was rinsed with DCM, collected by filtration, washed with DCM to yield the HCl salt of 7-(4-hydroxy-3,5-dimethylphenyl)-2,4-dimethoxy-1,6-naphthyridin-5(6H)-one (0.15 g, 59%) as a light yellow solid. Selected data: MS (ES) m/z: 327.06; MP >324° C. at decomposition (HCl salt).

### Example 14

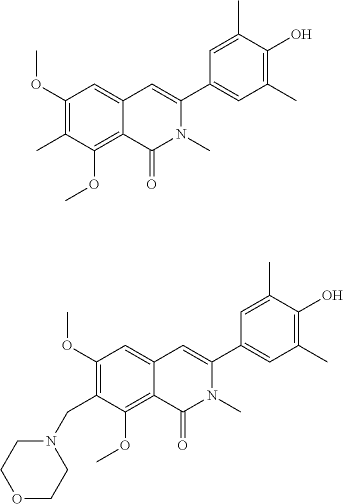 [SMILES: COc1cc2cc(-c3cc(C)c(O)c(C)c3)n(C)c(=O)c2c(OC)c1C ; COc1cc2cc(-c3cc(C)c(O)c(C)c3)n(C)c(=O)c2c(OC)c1CN1CCOCC1]

### 3-(4-hydroxy-3,5-dimethylphenyl)-6,8-dimethoxy-2,7-dimethylisoquinolin-1(2H)-one (left) and 3-(4-hydroxy-3,5-dimethylphenyl)-6,8-dimethoxy-2-methyl-7-(morpholinomethyl)isoquinolin-1(2H)-one (right)

Methyl acetoacetate (69.67 g, 0.6 mol) in dry THF (350 mL) was cooled to −5° C. and sodium hydride (60% in mineral oil, 24.5 g) was added at −5 to 0° C. in 30 min. Diketene (50.4 g) in dry THF (80 mL) was then added drop-wise at 5° C. over 20 min. The resulting solution was allowed to stir for 1 h at −5° C., after which it was allowed to warm to room temperature and stirred overnight. Acetic acid (35 mL) was added and the THF solvent was removed. Water (200 mL) and ethyl acetate (300 mL) was added to the residue, pH was adjusted to 5.0 by adding HCl solution. The organic layer was separated and washed with brine and dried over sodium sulfate. After column purification and recrystallization, compound A (methyl 2,4-dihydroxy-6-methylbenzoate) was obtained (yield: 26.6 g, 24.3%). Sodium hydride (11.2 g, 0.279 mol, 60% in mineral oil) was added to compound A (24.8 g, 0.136 mol) in DMF (150 mL). The reaction temperature was cooled to −30° C. and methyl iodide (21.3 mL, 0.341 mol) was added and the reaction was kept at room temperature overnight. Sodium iodide was filtered off and DMF was removed. The residue was mixed with water (100 mL) and extracted with ethyl acetate. The organic layer was further washed with brine and dried over sodium sulfate. The crude mixture was purified by column chromatography to yield compound B (11.40 g, 39.9%). To a solution of compound B (11.4 g, 0.054 mol) in dry CCl₄ (90 mL) was added N-bromosuccinimide (10.6 g, 0.0596 mol). The mixture was refluxed overnight. CCl₄ was removed. Water (100 mL) was added to the residue and the solid was filtered off and washed with water and a mixture of ethyl acetate (10 mL) and hexane (30 mL) to yield compound C (13.1 g, 83.9%). Compound C (12.5 g, 0.043 mol), chloromethyl methyl ether (81.0 g) and anhydrous zinc chloride (7.0 g, 0.0513 mol) was kept at room temperature overnight. Chloromethyl methyl ether was removed and the residue was mixed with water and the pH was adjusted to 7 by adding sodium bicarbonate. The mixture was extracted with ethyl acetate and the organic layer was washed with brine and dried over sodium sulfate. Compound D (7.39 g, 50.6%) was obtained after column chromatography. Compound D (7.39 g, 0.0218 mol), morpholine (7.62 g, 0.0875 mol), and anhydrous THF (20 mL) were stirred at room temperature overnight. The solvent was evaporated. Water and ethyl acetate were added to the residue, pH was adjusted to 9.0 with sodium bicarbonate. The organic layer was washed with brine and dried with sodium sulfate. Compound E (5.4 g, 63.8%) was obtained after column chromatography. The hydrogenation reaction was carried out at 50 psi with compound E (5.4 g, 0.0139 mol) in THF (100 mL) and triethyl amine (3.9 mL) with Pd/C (10%, 2.6 g) as a catalyst for 2 d. After the catalyst was filtered off, the organic layer was purified by column chromatography to yield compound F (3.20 g, 74.4%) and 1.1 g starting material E. Compound F (3.20 g, 0.0103 mol) was dissolved in ethanol (30 mL) and potassium hydroxide (2.31 g, 0.041 mol) in water (20 mL) was added and the reaction mixture was heated to 100° C. overnight. The solvent was removed, the pH was adjusted to 6.0 and the water was removed. The residue was further dried under high vacuum and the compound was extracted with ethanol to yield compound G (2.95 g, 99%). Compound G (2.80 g, 0.0095 mol) was mixed with thionyl chloride (7.0 mL, 0.0108 mol) and heated to reflux for 1 h. Excess thionyl chloride was removed and the residue was further dried under high vacuum and anhydrous THF (20 mL) was added and methylamine in THF (2.0 M, 30 mL) was added and the reaction was stirred for overnight. THF was removed and pH was adjusted to 8.0-9.0, the mixture was extracted with dichloromethane and dried over sodium sulfate to give compound H (2.50 g, 85.4%).

NaH (1.14 g, 0.0285 mol, 60% in mineral oil) was added to 4-hydroxy-3,5-dimethylbenzonitrile (4.0 g, 0.027 mol) in anhydrous DMF (20 mL), followed by benzyl bromide (3.27 mL, 0.027 mol). The reaction was kept at room temperature overnight. The reaction mixture was poured into water and the solid was filtered off and washed with hexane to yield compound I (5.7 g, 89%). Compound I was used for the next step without further purification.

n-BuLi (1.60 M, 3.3 mL) was added drop-wise to compound H (0.25 g, 0.81 mmol) in anhydrous THF (25 mL) at −10° C. The reaction mixture was kept at 0° C. for 1 h then the cool bath was removed and the reaction mixture was further stirred for 45 min. Compound I (0.192 g, 0.81 mmol) in anhydrous THF (5 mL) was added drop-wise at −10° C. and the reaction was further kept for 30 min; the reaction temperature was increased to room temperature and the reaction mixture was stirred for a further 1 h. Water (20 mL) was added and the mixture was extracted with ethyl acetate. The solvent was removed and the residue was treated with acetic acid at 65° C. for 30 min then purified by column chromatography to yield compound J (0.110 g, 25.9%). Product J (300 mg) in methanol (80 mL) and 10% Pd/C (100 mg) as catalyst was stirred under H₂ (50 psi) for 1 h. The catalyst was filtered off and the solvent was removed. The residue was purified by column chromatography (10% methanol in ethyl acetate) to yield 3-(4-hydroxy-3,5-dimethylphenyl)-6,8-dimethoxy-2,7-dimethylisoquinolin-1(2H)-one (60 mg, 29.8%) and 3-(4-hydroxy-3,5-dimethylphenyl)-6,8-dimethoxy-2-methyl-7-(morpholinomethyl)isoquinolin-1(2H)-one (40 mg, 16.8%). Selected data for 3-(4-hydroxy-3,5-dimethylphenyl)-6,8-dimethoxy-2,7-dimethylisoquinolin-1(2H)-one: MP 246-248° C. Selected data for 3-(4-hydroxy-3,5-dimethylphenyl)-6,8-dimethoxy-2-methyl-7-(morpholinomethyl)isoquinolin-1(2H)-one: MP 224-225° C.

### Example 15

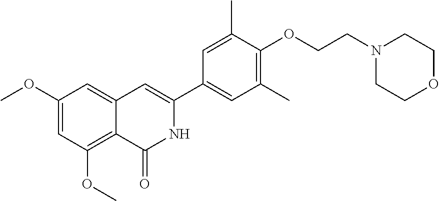 [SMILES: COc1cc(OC)c2c(=O)[nH]c(-c3cc(C)c(OCCN4CCOCC4)c(C)c3)cc2c1]

### 3-(3,5-dimethyl-4-(2-morpholinoethoxy)phenyl)-6,8-dimethoxyisoquinolin-1(2H)-one

(3,5-Dimethoxy-phenyl)-acetic acid (10.0 g, 51 mmol) was dissolved in anhydrous methanol (100 mL) and H₂SO₄ (1 mL) was added drop-wise. The reaction mixture was refluxed overnight and cooled to room temperature. The solvent was removed and the residue was dissolved in ethyl acetate and washed with a NaHCO₃ solution, water and dried (Na₂SO₄). The solvent was evaporated in vacuo to obtain (3,5-dimethoxy-phenyl)-acetic acid methyl ester in 97% (10.4 g) yield. To a solution of (3,5-dimethoxy-phenyl)-acetic acid methyl ester (10.4 g, 49.5 mmol) in dimethyl formamide (40 mL), POCl₃ (5.4 mL, 59.37 mmol) was added at 55° C. After the addition, the reaction mixture was heated at 100° C. for 10 min and then stirred at room temperature overnight. The reaction mixture was poured into ice-water and extracted with ethyl acetate, washed with water, brine, dried over anhydrous Na₂SO₄ and evaporated in vacuo to obtain (2-formyl-3,5-dimethoxy-phenyl)-acetic acid methyl ester (10.0 g, 85%). (2-Formyl-3,5-dimethoxy-phenyl)-acetic acid methyl ester (5.0 g, 21 mmol) was dissolved in CH₃CN (100 mL), NaH₂PO₄ (0.655 g, 5.46 mmol) in water (2 mL) and 30% H₂O₂ (2.3 mL, 21 mmol). The reaction mixture was cooled to 0° C. and a solution of NaO₂Cl (2.65 g, 29.4 mmol) in water (5 mL) was added. The reaction mixture was stirred at room temperature for 4 h before being quenched by the addition of Na₂SO₃ solution. The mixture was acidified with 2 N HCl and extracted with ethyl acetate. The solvent was evaporated in vacuo to obtain 2,4-dimethoxy-6-methoxycarbonylmethyl-benzoic acid (5.25 g, 98%). To a solution of 2,4-dimethoxy-6-methoxycarbonylmethyl-benzoic acid (5.25 g, 20.6 mmol) in methanol (50 mL), a solution of NaOH (4.12 g, 103 mmol) in water (20 mL) was added and the reaction mixture was allowed to stir at room temperature for 3 h. The solvent was removed, diluted with water and acidified with 2 N HCl. The compound was extracted with ethyl acetate, washed with water, brine, dried over anhydrous Na₂SO₄ and evaporated in vacuo to obtain 2-carboxymethyl-4,6-dimethoxy-benzoic acid (4.65 g, 94%). To a suspension of 2-carboxymethyl-4,6-dimethoxy-benzoic acid (4.65 g, 19.4 mmol) in toluene (50 mL) was added acetic anhydride (2.01 mL, 21.3 mmol) and the reaction mixture was heated to reflux for 2 h. After cooling to 0° C., the precipitated solid was filtered off and washed with heptane and hexane to obtain 6,8-dimethoxy-isochroman-1,3-dione (3.56 g, 83%).

To a solution of 3,5-dimethyl-4-hydroxy-benzoic acid (3.0 g, 18.05 mmol) in pyridine (7 mL) was added acetic anhydride (2.05 mL, 21.66 mmol) and the reaction mixture was stirred at room temperature for 16 h. Water was added and the mixture was extracted with ethyl acetate, washed with water, brine and dried over anhydrous Na₂SO₄. The solvent was evaporated in vacuo to obtain 4-acetoxy-3,5-dimethyl-benzoic acid (3.52 g, 94%). To a solution of 4-acetoxy-3,5-dimethyl-benzoic acid (6.02 g, 28.91 mmol) in CH₂Cl₂ (80 mL), oxalyl chloride (5.04 mL, 57.83 mmol) was added slowly, followed by a drop of dimethyl formamide. The reaction mixture was stirred at room temperature for 2 h. The solvent was removed and acetic acid 4-chlorocarbonyl-2,6-dimethyl-phenyl ester was dried under vacuum (6.37 g, 97%). To a solution of N,N,N,N-tetramethyl guanidine (2.77 mL, 22.078 mmol) in CH₃CN (50 mL), a solution of 6,8-dimethoxy-isochroman-1,3-dione (4.46 g, 20.07 mmol) in CH₃CN (100 mL) was added slowly at <0° C. (bath temperature −20° C.) in 30 min. Then, Et₃N was added in one portion, followed by a solution of acetic acid 4-chlorocarbonyl-2,6-dimethyl-phenyl ester (6.37 g, 28.1 mmol) in CH₃CN (50 mL) and stirred for 30 min. at <0° C. The reaction mixture was stirred at room temperature for 16 h, then heated to reflux for 3 h. After cooling to room temperature, the reaction mixture was quenched with 1 N HCl. The precipitated solid was filtered off to give a mixture of acetic acid 4-(6,8-dimethoxy-1,3-dioxo-isochroman-4-carbonyl)-2,6-dimethyl-phenyl ester and acetic acid 4-(6,8-dimethoxy-1-oxo-1H-isochromen-3-yl)-2,6-dimethyl-phenyl ester (6.0 g).

The above mixture (6.0 g) was dissolved in H₂SO₄ (30%, 30 mL) and heated at 100° C. for 2 h. The reaction mixture was cooled to room temperature and the precipitated solid was filtered off to obtain a mixture of acetic acid 4-(6,8-dimethoxy-1-oxo-1H-isochromen-3-yl)-2,6-dimethyl-phenyl ester and 3-(4-hydroxy-3,5-dimethyl-phenyl)-6,8-dimethoxy-isochromen-1-one (5.5 g). The above mixture (5.5 g) was dissolved in methanol (30 mL), K₂CO₃ (3.09 g, 22.4 mmol) and water (10 mL) were added and the reaction mixture was stirred at room temperature for 6 h. The solvent was removed and acidified with dilute HCl. The compound was extracted with ethyl acetate, washed with water, brine and dried over anhydrous Na₂SO₄. The solvent was evaporated in vacuo to leave a residue which was purified by chromatography (silica gel, 230-250 mesh; 2% methanol in dichloromethane) to obtain 3-(4-hydroxy-3,5-dimethyl-phenyl)-6,8-dimethoxy-isochromen-1-one. The yield was 1.462 g.

To a solution of 3-(4-hydroxy-3,5-dimethyl-phenyl)-6,8-dimethoxy-isochromen-1-one (0.875 g, 2.68 mmol) in DMF (5 mL), NaH (0.129 g, 3.22 mmol) was added and the mixture was stirred for 1 h. To the reaction mixture was added 1-chloro-2-iodo-ethane (1.23 mL, 13.4 mmol) and stirring was continued for 16 h. Then the reaction mixture was heated at 80° C. before being quenched with 1 N HCl at room temperature. The crude was purified by column chromatography (silica gel, 230-250 mesh; 2% methanol in dichloromethane). The yield was 0.36 g (35%). The compound 3-[4-(2-chloro-ethoxy)-3,5-dimethyl-phenyl]-6,8-dimethoxy-isochromen-1-one (0.36 g, 0.927 mmol) was dissolved in DMSO (5 mL), morpholine (0.4 mL, 4.63 mmol) and Et₃N (0.64 mL, 4.63 mmol) were added. The reaction mixture was heated at 110° C. for 16 h before being cooled to room temperature. Water was added and the compound was extracted with ethyl acetate. The solvent was evaporated in vacuo to leave a residue, which was purified by chromatography. The yield was 0.128 g (31%). The compound 3-[3,5-dimethyl-4-(2-morpholin-4-yl-ethoxy)-phenyl]-6,8-dimethoxy-isochromen-1-one (0.128 g, 0.29 mmol) and NH₃ (2.0 M solution in ethanol, 30 mL) were mixed in a steel bomb and heated at 130° C. for 16 h. The solvent was removed and the crude compound was purified by chromatography (silica gel, 230-250 mesh). The compound was then converted into the HCl salt of 3-(3,5-dimethyl-4-(2-morpholinoethoxy)phenyl)-6,8-dimethoxyisoquinolin-1(2H)-one (84 mg, 66%). Selected data: MP 196-198° C. (HCl salt).

### Example 16

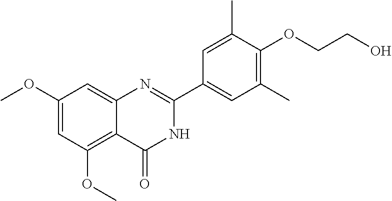 [SMILES: COc1cc(OC)c2c(=O)[nH]c(-c3cc(C)c(OCCO)c(C)c3)nc2c1]

### 2-(4-(2-hydroxyethoxy)-3,5-dimethylphenyl)-5,7-dimethoxyquinazolin-4(3H)-one

A solution of 2-amino-4,6-dimethoxybenzamide (0.60 g, 3.06 mmol) and 4-[2-(tert-butyldimethylsilanoxy)ethoxy]-3,5-dimethylbenzaldehyde (0.856 g, 2.78 mmol) in N,N-dimethyl formamide (20 mL) was stirred at 70° C. for 1 h. Iodine (0.846 g, 3.33 mmol) and potassium carbonate (0.384 g, 2.78 mmol) were added and the reaction mixture was stirred at 70° C. for 16 h. The reaction mixture was poured into ice, and extracted with ethyl acetate. The organic layer was washed with water, brine, and dried over anhydrous Na₂SO₄. Removal of the solvent gave the crude product which was purified by column chromatography to give 2-(4-(2-hydroxyethoxy)-3,5-dimethylphenyl)-5,7-dimethoxyquinazolin-4(3H)-one (444 mg, 39%) as a white solid. Selected data: 229-231° C.

Alternatively, 2-(4-(2-hydroxyethoxy)-3,5-dimethylphenyl)-5,7-dimethoxyquinazolin-4(3H)-one can be synthesized by the following method. In a 2 L dry round-bottom flask with a reflux condenser and magnetic stirrer was placed 3,5-dimethyl-4-hydroxy benzaldehyde (26.9 g, 0.179 mol) in ethanol (350 mL). 2-chloroethanol (87.6 g, 1.074 mol) and K₂CO₃ (99 g, 0.716 mol) were added and the reaction mixture was heated to reflux for 24 h. The reaction mixture was cooled to room temperature and filtered. The solvent was removed under reduced pressure. The crude product was diluted with ethyl acetate and the organic layer was washed with water, brine, and dried over Na₂SO₄. Upon removal of solvent it gave 45 g of crude product. The crude product was purified by column chromatography (silica gel 230-400 mesh; 50% ethyl acetate in hexane as eluent) to give 33.3 g (95%) of product. To a solution of 2-amino-4,6-dimethoxy-benzamide (33.45 g, 0.170 mol) and 4-(2-hydroxy ethoxy)-3,5-dimethyl benzaldehyde (33.3 g, 0.170 mol) in N,N-dimethyl acetamide (300 mL), NaHSO₃ (33.3 g, 0.187 mol) and p-TSA (3.2 g, 17.1 mmol) were added and the reaction mixture was heated at 150° C. for 14 h. The reaction was cooled to room temperature. The solvent was removed under reduced pressure. The residue was diluted with water and stirred for 30 min at room temperature. The solids separated were filtered and dried to give crude product. The crude product was purified by column chromatography (silica gel 230-400 mesh; 5% methanol in CH₂Cl₂ as eluent) to give 2-(4-(2-hydroxyethoxy)-3,5-dimethylphenyl)-5,7-dimethoxyquinazolin-4(3H)-one (33 g, 52%).

### Example 17

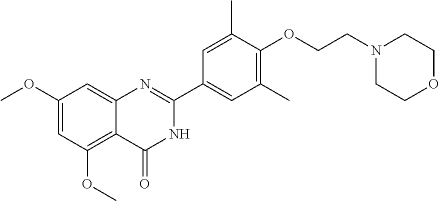 [SMILES: COc1cc(OC)c2c(=O)[nH]c(-c3cc(C)c(OCCN4CCOCC4)c(C)c3)nc2c1]

### 2-(3,5-dimethyl-4-(2-morpholinoethoxy)phenyl)-5,7-dimethoxyquinazolin-4(3H)-one

In a 250 mL round-bottomed flask were placed 3,5-dimethyl-4-hydroxy benzaldehyde (4.0 g, 26.7 mmol), Ph₃P (15.38 g, 58.66 mmol), di-isopropylethylamine (13.78 g, 106.7 mmol) and 2-morpholin-4-yl-ethanol (7.69, 58.7 mmol) in THF (100 mL), then DEAD (11.1 g, 64 mmol) was added drop-wise at room temperature. The reaction mixture was stirred for 3 d at room temperature and water was added and extracted with ethyl acetate. The combined organic layers were washed with water, brine and dried over Na₂SO₄ and concentrated to give crude product. The crude product was purified by column chromatography to give the B-ring building block (3.0 g, 43%).

To a solution of 2-amino-4,6-dimethoxybenzamide (451 mg, 2.3 mmol) and 3,5-dimethyl-4-(2-morpholin-4yl-ethoxy)-benzaldehyde (550 mg, 2.09 mmol) in N,N-dimethyl formamide (20 mL), iodine (636 mg, 2.5 mmol) and potassium carbonate (288 mg, 2.09 mmol) were added and the reaction mixture was stirred at 70° C. for 48 h. The reaction mixture was poured into ice. The mixture was extracted with ethyl acetate. The organic layer was washed with water, brine and dried over anhydrous Na₂SO₄. Removal of the solvent gave the crude product was purified by column chromatography and converted to the hydrochloride salt of 2-(3,5-dimethyl-4-(2-morpholinoethoxy)phenyl)-5,7-dimethoxyquinazolin-4(3H)-one (40 mg, 4%) as an off-white solid. Selected data: MS (ES) m/z: 440.1; MP 185-187° C. (HCl salt).

### Example 18

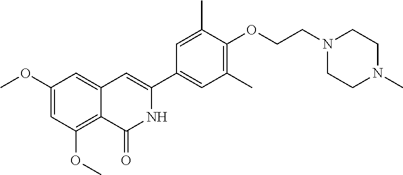

### 3-(3,5-dimethyl-4-(2-(4-methylpiperazin-1-yl)ethoxy)phenyl)-6,8-dimethoxyisoquinolin-1(2H)-one

The compound 3-[4-(2-chloro-ethoxy)-3,5-dimethyl-phenyl]-6,8-dimethoxy-isochromen-1-one (298 mg, 0.767 mmol) was dissolved in DMSO (5 mL) and N-methyl piperazine (388 mg, 3.83 mmol) and Et₃N (392 mg, 3.83 mmol) were added. The reaction mixture was heated at 110° C. for 16 h before being cooled to room temperature. Water was added and the mixture was extracted with ethyl acetate. The solvent was evaporated in vacuo to leave a residue which was purified by column chromatography. The yield was 60 mg (17%). The compound 3-[3,5-dimethyl-4-(2-(4-methyl piperazin-1-yl-ethoxy)-phenyl]-6,8-dimethoxy-isochromen-1-one (60 mg, 0.13 mmol) and NH₃ (2.0 M solution in ethanol, 20 mL) were mixed in a steel bomb and heated at 130° C. for 16 h. The solvent was removed and the crude compound was purified by column chromatography. The compound was then converted to the hydrochloride salt of 3-(3,5-dimethyl-4-(2-(4-methylpiperazin-1-yl)ethoxy)phenyl)-6,8-dimethoxyisoquinolin-1(2H)-one (40 mg, 62%), an off-white solid. Selected data: MS (ES) m/z: 452.1; MP 195-198° C. (HCl salt).

### Example 19

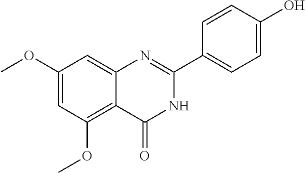 [SMILES: COc1cc(OC)c2c(=O)[nH]c(-c3ccc(O)cc3)nc2c1]

### 2-(4-hydroxyphenyl)-5,7-dimethoxyquinazolin-4(3H)-one

2-Amino-4,6-dimethoxy-benzamide (328 mg, 1.67 mmol), 4-hydroxybenzaldehyde (204 mg, 1.67 mmol), K₂CO₃ (231 mg, 1.67 mmol) and I₂ (508 mg, 2.0 mmol) were mixed in DMF (10 mL) and the reaction mixture was heated at 70° C. for 5 h. It was cooled to room temperature and poured into crushed ice. The solid was collected and purified by column chromatography (silica gel 230-400 mesh; 5% methanol in CH₂Cl₂ as eluent) to give 2-(4-hydroxyphenyl)-5,7-dimethoxyquinazolin-4(3H)-one (60 mg, 12%), as an off-white solid. Selected data: MS (m/z): 299.05; MP 303-305° C.

### Example 20

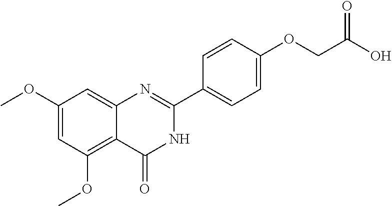 [SMILES: COc1cc(OC)c2c(=O)[nH]c(-c3ccc(OCC(=O)O)cc3)nc2c1]

### 2-(4-(5,7-dimethoxy-4-oxo-3,4-dihydroquinazolin-2-yl)phenoxy)acetic acid

2-(4-(5,7-Dimethoxy-4-oxo-3,4-dihydroquinazolin-2-yl)phenoxy)acetic acid was synthesized from 2-amino-4,6-dimethoxy-benzamide and (4-formyl phenoxy)acetic acid, using the method described for 2-(4-hydroxyphenyl)-5,7-dimethoxyquinazolin-4(3H)-one. 2-(4-(5,7-Dimethoxy-4-oxo-3,4-dihydroquinazolin-2-yl)phenoxy)acetic acid (135 mg, 21%) was isolated as an off-white solid. Selected data: MS (m/z): 357.04; MP 287-290° C.

### Example 21

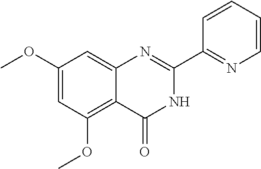 [SMILES: COc1cc(OC)c2c(=O)[nH]c(-c3ccccn3)nc2c1]

### 5,7-dimethoxy-2-(pyridin-2-yl)quinazolin-4(3H)-one

To a solution of 2-amino-4,6-dimethoxybenzamide (0.15 g, 0.764 mmol) in N,N-dimethyl acetamide (5 mL) were added 2-pyridine carboxaldehyde (0.082 g, 0.764 mmol), sodium hydrogen sulphite (58.5%, 0.15 g, 0.84 mmol), and p-toluenesulfonic acid (15 mg, 0.0764 mmol). The reaction mixture was stirred at 150° C. overnight. The mixture was cooled to room temperature. Water (40 mL) was added and the reaction mixture was extracted with dichloromethane (2×50 mL). The combined organic layers were washed with water and dried over anhydrous Na₂SO₄. The solvent was removed and the crude compound was purified by column chromatography (silica gel 230-400 mesh; 1% methanol in CH₂Cl₂ as eluent) to give 5,7-dimethoxy-2-(pyridin-2-yl)quinazolin-4(3H)-one (0.077 g, 36%) as a white solid. 5,7-dimethoxy-2-(pyridin-2-yl)quinazolin-4(3H)-one was converted to the corresponding hydrochloride. Selected data: MS (m/z): 284.0; MP 215-217° C. (hydrochloride).

### Example 22

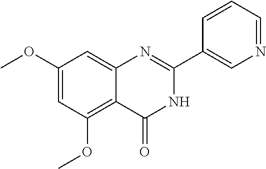 [SMILES: COc1cc(OC)c2c(=O)[nH]c(-c3cccnc3)nc2c1]

### 5,7-dimethoxy-2-(pyridin-3-yl)quinazolin-4(3H)-one

5,7-Dimethoxy-2-(pyridin-3-yl)quinazolin-4(3H)-one was synthesized from 2-amino-4,6-dimethoxybenzamide and 3-pyridine carboxaldehyde, using the method described for 5,7-dimethoxy-2-(pyridin-2-yl)quinazolin-4(3H)-one. 5,7-Dimethoxy-2-(pyridin-3-yl)quinazolin-4(3H)-one (105 mg, 48%) was isolated as a white solid. Selected data: MS (m/z): 284.0; MP 257-259° C. (hydrochloride).

### Example 23

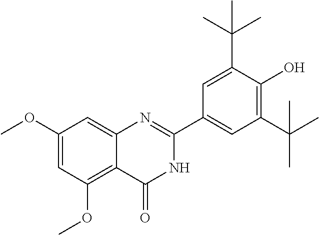 [SMILES: COc1cc(OC)c2c(=O)[nH]c(-c3cc(C(C)(C)C)c(O)c(C(C)(C)C)c3)nc2c1]

### 2-(3,5-di-tert-butyl-4-hydroxyphenyl)-5,7-dimethoxyquinazolin-4(3H)-one

2-(3,5-di-tert-butyl-4-hydroxyphenyl)-5,7-dimethoxyquinazolin-4(3H)-one was synthesized from 2-amino-4,6-dimethoxybenzamide and 3,5-di-tert-butyl-4-hydroxybenzaldehyde, using the method described for 5,7-dimethoxy-2-(pyridin-2-yl)quinazolin-4(3H)-one. 2-(3,5-di-tert-butyl-4-hydroxyphenyl)-5,7-dimethoxyquinazolin-4(3H)-one (130 mg, 41%) was isolated as a light yellow solid. Selected data: MS (m/z): 411.17; MP 229.7-230.5° C.

### Example 24

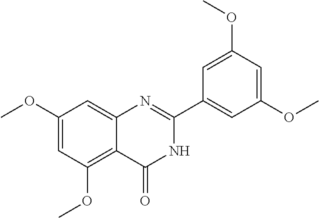

### 2-(3,5-dimethoxyphenyl)-5,7-dimethoxyquinazolin-4(3H)-one

2-(3,5-dimethoxyphenyl)-5,7-dimethoxyquinazolin-4(3H)-one was synthesized from 2-amino-4,6-dimethoxybenzamide and 3,5-dimethoxybenzaldehyde, using the method described for 5,7-dimethoxy-2-(pyridin-2-yl)quinazolin-4(3H)-one. 2-(3,5-dimethoxyphenyl)-5,7-dimethoxyquinazolin-4(3H)-one (120 mg, 46%) was isolated as a yellow solid. Selected data: MS (m/z): 343.05; MP 270-272° C.

### Example 25

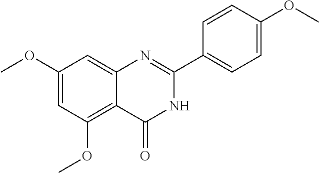

### 5,7-dimethoxy-2-(4-methoxyphenyl)quinazolin-4(3H)-one

5,7-Dimethoxy-2-(4-methoxyphenyl)quinazolin-4(3H)-one was synthesized from 2-amino-4,6-dimethoxybenzamide and 4-methoxy benzaldehyde, using the method described for 5,7-dimethoxy-2-(pyridin-2-yl)quinazolin-4(3H)-one. 5,7-Dimethoxy-2-(4-methoxyphenyl)quinazolin-4(3H)-one (106 mg, 44%) was isolated as an off-white solid. Selected data: MS (m/z): 312.99; MP 276-277° C.

### Example 26

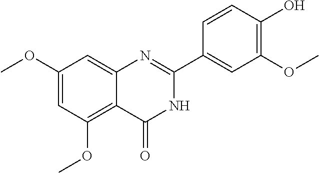

### 2-(4-hydroxy-3-methoxyphenyl)-5,7-dimethoxyquinazolin-4(3H)-one

2-(4-Hydroxy-3-methoxyphenyl)-5,7-dimethoxyquinazolin-4(3H)-one was synthesized from 2-amino-4,6-dimethoxybenzamide and 4-hydroxy-3-methoxybenzaldehyde, using the method described for 5,7-dimethoxy-2-(pyridin-2-yl)quinazolin-4(3H)-one. 2-(4-Hydroxy-3-methoxyphenyl)-5,7-dimethoxyquinazolin-4(3H)-one (90 mg, 36%) was isolated as a white solid. Selected data: MS (m/z): 329.06; MP 294-296° C.

### Example 27

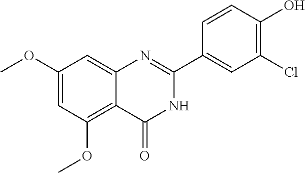 [SMILES: COc1cc(OC)c2c(=O)[nH]c(-c3ccc(O)c(Cl)c3)nc2c1]

### 2-(3-chloro-4-hydroxyphenyl)-5,7-dimethoxyquinazolin-4(3H)-one

2-(3-Chloro-4-hydroxyphenyl)-5,7-dimethoxyquinazolin-4(3H)-one was synthesized from 2-amino-4,6-dimethoxybenzamide and 3-chloro-4-hydroxybenzaldehyde, using the method described for 5,7-dimethoxy-2-(pyridin-2-yl)quinazolin-4(3H)-one. 2-(3-Chloro-4-hydroxyphenyl)-5,7-dimethoxyquinazolin-4(3H)-one (75 mg, 30%) was isolated as a yellow solid. Selected data: MS (m/z): 333.03; MP 279-281° C.

### Example 28

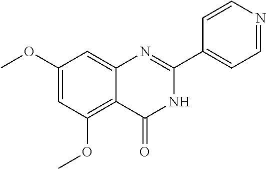 [SMILES: COc1cc(OC)c2c(=O)[nH]c(-c3ccncc3)nc2c1]

### 5,7-dimethoxy-2-(pyridin-4-yl)quinazolin-4(3H)-one

5,7-Dimethoxy-2-(pyridin-4-yl)quinazolin-4(3H)-one was synthesized from 2-amino-4,6-dimethoxybenzamide and 4-pyridine carboxaldehyde, using the method described for 5,7-dimethoxy-2-(pyridin-2-yl)quinazolin-4(3H)-one. 5,7-Dimethoxy-2-(pyridin-4-yl)quinazolin-4(3H)-one (142 mg, 63%) was isolated as a pale brown solid and then converted to the corresponding hydrochloride (yellow solid). Selected data: MS (m/z): 284.06; MP 294-295° C. (hydrochloride).

### Example 29

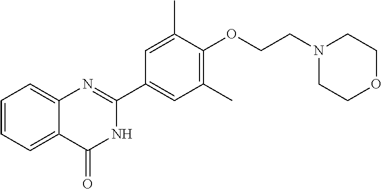 [SMILES: Cc1cc(-c2nc3ccccc3c(=O)[nH]2)cc(C)c1OCCN1CCOCC1]

### 2-(3,5-dimethyl-4-(2-morpholinoethoxy)phenyl)quinazolin-4(3H)-one

To a solution of 4-hydroxy-3,5-dimethyl benzaldehyde (3.0 g, 20 mmol) in anhydrous THF (100 mL), triphenyl phosphene (10.49 g, 40 mmol), 4-(2-hydroxyethyl)morpholine (5.25 g, 40 mmol) and N,N-diisopropylethylamine (7.76 g, 60 mmol) were added. To this stirred solution was added diethylazodicarboxylate (6.97 g, 40 mmol). The reaction mixture was stirred at room temperature overnight under nitrogen and diluted with ethyl acetate (200 mL). The organic layer was washed with water and brine and dried over anhydrous Na₂SO₄. The solvent was removed under reduced pressure. The crude material was purified by column chromatography (silica gel 230-400 mesh; 0-3% methanol in CH₂Cl₂ as eluent) to give 3,5-dimethyl-4-(2-morpholin-4-yl-ethoxy)benzaldehyde (1.66 g, 32%) as an oil.

To a solution of 2-amino benzamide (136 mg, 1.0 mmol) in N,N-dimethyl acetamide (5 mL) were added 3,5-dimethyl-4-(2-morpholin-4-yl-ethoxy)benzaldehyde (263 mg, 1.0 mmol), sodium hydrogen sulphite (58.5%) (196 mg, 1.1 mmol) and p-toluenesulfonic acid (19 mg, 0.1 mmol). The reaction mixture was stirred at 150° C. overnight. Water (40 mL) was added. The formed solid was filtered off, washed with water and a small amount of methanol and dried under vacuum to give the title compound (190 mg, 50%) as an off-white solid. To a solution of the above compound (174 mg, 0.458 mmol) in 2:1 anhydrous CH₂Cl₂-methanol (15 mL) was added 1.0 M solution of hydrogen chloride in ether (1.5 mL) and the reaction mixture was stirred at room temperature for 16 h. The solvent was removed under reduced pressure. The residue was triturated with 10% methanol in anhydrous ether to give the hydrochloride of 2-(3,5-dimethyl-4-(2-morpholinoethoxy)phenyl)quinazolin-4(3H)-one (187 mg, 98%), as an off-white solid. Selected data: MS (ES) m/z: 380.10; MP 300-302° C. (hydrochloride).

### Example 30

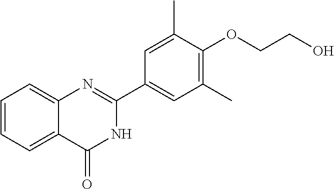 [SMILES: Cc1cc(-c2nc3ccccc3c(=O)[nH]2)cc(C)c1OCCO]

### 2-(4-(2-hydroxyethoxy)-3,5-dimethylphenyl)quinazolin-4(3H)-one

A mixture of anthranilamide (0.15 g, 1.10 mmol), 4-[2(tert-butyl-dimethyl-silanyloxy)-ethoxy]-3,5-dimethyl-benzaldehyde (0.340 g, 1.101 mmol), sodium hydrogensulfite (0.126 g, 1.101 mmol) and p-toluenesulfonic acid (20 mg) in N,N-dimethyl acetamide (5 mL) was stirred at 150° C. for 3 h under nitrogen. The reaction mixture was cooled to room temperature and diluted with water (20 mL). The solid was collected by filtration, washed with water (10 mL×3) and dried under high vacuum to provide desired compound (328 mg, 70%), as a white solid. A solution of the above described compound (0.316 g, 0.745 mmol) in THF (3 mL) was cooled to 0° C. under nitrogen and TBAF (1.5 mL, 1.49 mmol) was added, followed by stirring at room temperature for 1 h. The reaction mixture was diluted with cold water (30 mL), the white precipitate was filtered off, washed with water (15 mL×3) and MeOH (20 mL×3) and dried under high vacuum, to afford 2-(4-(2-hydroxyethoxy)-3,5-dimethylphenyl)quinazolin-4(3H)-one (150 mg, 65%), as a white solid. Selected data: MS (ES) m/z: 311.04; MP 260-261° C.

### Example 31

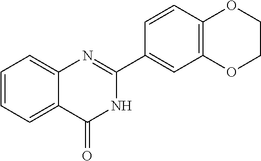 [SMILES: O=c1[nH]c(-c2ccc3c(c2)OCCO3)nc2ccccc12]

### 2-(2,3-dihydrobenzo[b][1,4]dioxin-6-yl)quinazolin-4(3H)-one

2-(2,3-Dihydrobenzo[b][1,4]dioxin-6-yl)quinazolin-4(3H)-one was synthesized from anthranilamide and 4-(pyrimidin-2-yloxy)-benzaldehyde, using the method described for 5,7-dimethoxy-2-(pyridin-2-yl)quinazolin-4(3H)-one. 2-(2,3-Dihydrobenzo[b][1,4]dioxin-6-yl)quinazolin-4(3H)-one (222 mg, 72%) was isolated as a light beige solid. Selected data: MS (m/z): 280.98; MP 267-268° C. (decomposed).

### Example 32

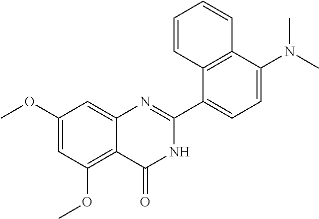

### 2-(4-(dimethylamino)naphthalen-1-yl)-5,7-dimethoxyquinazolin-4(3H)-one

2-(4-(Dimethylamino)naphthalen-1-yl)-5,7-dimethoxyquinazolin-4(3H)-one was synthesized from 2-amino-4,6-dimethoxybenzamide and 4-dimethylamino-1-naphthaldehyde, using the method described for 5,7-dimethoxy-2-(pyridin-2-yl)quinazolin-4(3H)-one. 2-(4-(Dimethylamino)naphthalen-1-yl)-5,7-dimethoxyquinazolin-4(3H)-one (75 mg, 26%) was isolated as a yellow solid. Selected data: MS (m/z): 376.07; MP 269-271° C.

### Example 33

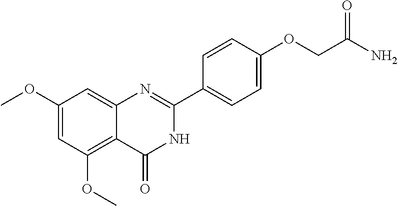 [SMILES: COc1cc(OC)c2c(=O)[nH]c(-c3ccc(OCC(N)=O)cc3)nc2c1]

### 2-(4-(5,7-dimethoxy-4-oxo-3,4-dihydroquinazolin-2-yl)phenoxy)acetamide

2-Amino-4,6-dimethoxy-benzamide (150 mg, 0.764 mmol), 2-(4-formyl-phenoxy)-acetamide (137 mg, 0.764 mmol), sodium hydrogen sulfite (150 mg, 58.5%) and p-toluenesulfonic acid monohydrate (15 mg) in N,N-dimethyl acetamide (15 mL) were heated to 150° C. overnight. N,N-dimethyl acetamide was removed under vacuum and the residue was poured into water (50 mL). The solid was filtered off and washed with methanol to yield 2-(4-(5,7-dimethoxy-4-oxo-3,4-dihydroquinazolin-2-yl)phenoxy)acetamide (74 mg, 27.2%). Selected data: MS (m/z): 356.09; MP 309-311° C. HPLC purity: 88.57%.

### Example 34

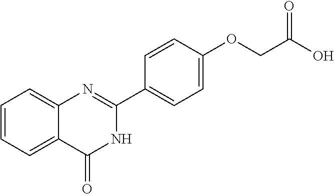 [SMILES: O=C(O)COc1ccc(-c2nc3ccccc3c(=O)[nH]2)cc1]

### 2-(4-(4-oxo-3,4-dihydroquinazolin-2-yl)phenoxy)acetic acid

2-(4-(4-Oxo-3,4-dihydroquinazolin-2-yl)phenoxy)acetic acid was synthesized from anthranilamide and 4-formyl phenoxy acetic acid, using the method described for 5,7-dimethoxy-2-(pyridin-2-yl)quinazolin-4(3H)-one. 2-(4-(4-Oxo-3,4-dihydroquinazolin-2-yl)phenoxy)acetic acid (800 mg, 73%) was isolated as a white solid. Selected data: MS (m/z): 296.98; MP 285-287° C.

### Example 35

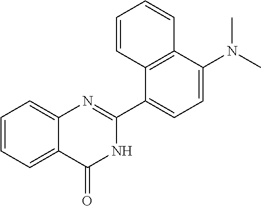 [SMILES: CN(C)c1ccc(-c2nc3ccccc3c(=O)[nH]2)c2ccccc12]

### 2-(4-(dimethylamino)naphthalen-1-yl)quinazolin-4(3H)-one

2-(4-(Dimethylamino)naphthalen-1-yl)quinazolin-4(3H)-one was synthesized from anthranilamide and 4-dimethylamino-naphthalene-1-carbaldehyde, using the method described for 5,7-dimethoxy-2-(pyridin-2-yl)quinazolin-4(3H)-one. 2-(4-(Dimethylamino)naphthalen-1-yl)quinazolin-4(3H)-one (240 mg, 69%) was isolated as a pale yellow solid. Selected data: MS (m/z): 316.08; MP 224-226° C.

### Example 36

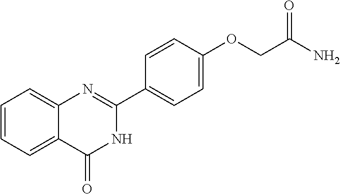 [SMILES: NC(=O)COc1ccc(-c2nc3ccccc3c(=O)[nH]2)cc1]

### 2-(4-(4-oxo-3,4-dihydroquinazolin-2-yl)phenoxy)acetamide

2-(4-(4-Oxo-3,4-dihydroquinazolin-2-yl)phenoxy)acetamide was synthesized from anthranilamide and 2-(4-formyl-phenoxy)-acetamide, using the method described for 5,7-dimethoxy-2-(pyridin-2-yl)quinazolin-4(3H)-one. 2-(4-(4-Oxo-3,4-dihydroquinazolin-2-yl)phenoxy)acetamide (183 mg, 56%) was isolated as a light beige solid. Selected data: MS (m/z): 295.97; MP 277.5-278.5° C.

### Example 37

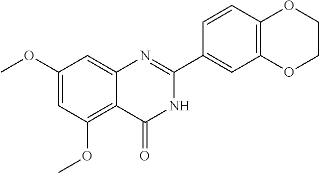 [SMILES: COc1cc(OC)c2c(=O)[nH]c(-c3ccc4c(c3)OCCO4)nc2c1]

### 2-(2,3-dihydrobenzo[b][1,4]dioxin-6-yl)-5,7-dimethoxyquinazolin-4(3H)-one

2-(2,3-Dihydrobenzo[b][1,4]dioxin-6-yl)-5,7-dimethoxyquinazolin-4(3H)-one was synthesized from 2-amino-4,6-dimethoxybenzamide and 2,3-dihydro-benzo[1,4]dioxine-6-carbaldehyde, using the method described for 5,7-dimethoxy-2-(pyridin-2-yl)quinazolin-4(3H)-one. 2-(2,3-Dihydrobenzo[b][1,4]dioxin-6-yl)-5,7-dimethoxyquinazolin-4(3H)-one (120 mg, 46%) was isolated as a yellow solid. Selected data: MS (m/z): 341.03; MP 307.5-309.6° C.

### Example 38

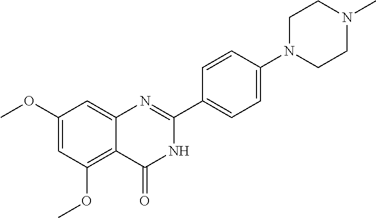

### 5,7-dimethoxy-2-(4-(4-methylpiperazin-1-yl)phenyl)quinazolin-4(3H)-one

A solution of 4-(4-formyl-phenyl)-piperazine-1-carboxylic acid tert-butyl ester (1.3 g, 4.47 mmol) in THF (50 mL) was mixed with LAH (0.7 g, 17.87 mmol) and stirred at reflux for 14 h. The reaction was quenched at room temperature by adding KOH aqueous (14 N, 20 mL). The supernatant was decanted and combined with DCM washings, then diluted with water (50 mL). The mixture was extracted with DCM (3×50 mL) followed by concentration using a rotary evaporator to give [4-(4-methyl-piperazin-1-yl)-phenyl]-methanol (0.82 g, 89%). To a solution of DMSO (0.56 mL, 7.96 mmol) in DCM (50 mL) at −78° C. was added oxalyl chloride (0.7 mL, 7.96 mmol) and the resulting mixture was stirred at −78° C. for 0.5 h. A solution of [4-(4-methyl-piperazin-1-yl)-phenyl]-methanol (0.82 g, 3.98 mmol) in DCM (20 mL) was slowly added. The reaction was stirred at −78° C. for 1.5 h. Triethylamine (1.7 mL, 11.94 mmol) was added and the reaction was allowed to gradually warm up to room temperature. After stirring for 4 h the reaction was quenched by adding sodium bicarbonate aqueous (1 N, 50 mL). The mixture was extracted with DCM (3×50 mL) followed by concentration to afford a residue, which was further purified by column chromatography to yield 4-(4-methyl-piperazin-1-yl)-benzaldehyde (0.5 g, 61%).

5,7-Dimethoxy-2-(4-(4-methylpiperazin-1-yl)phenyl)quinazolin-4(3H)-one was synthesized from 2-amino-4,6-dimethoxybenzamide and 4-(4-methyl-piperazin-1-yl)-benzaldehyde, using the method described for 5,7-dimethoxy-2-(pyridin-2-yl)quinazolin-4(3H)-one. 5,7-Dimethoxy-2-(4-(4-methylpiperazin-1-yl)phenyl)quinazolin-4(3H)-one (120 mg, 41%) was converted to the corresponding hydrochloride (a yellow solid). Selected data: MS (m/z): 381.11; MP 252.4-254.2° C. (di-hydrochloride).

### Example 39

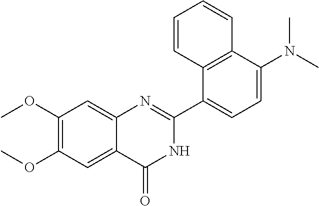

### 2-(4-(dimethylamino)pyridin-1-yl)-6,7-dimethoxyquinazolin-4(3H)-one

A solution of 4,5-dimethoxy-2-nitrobenzamide (10 g, 44.24 mmol) in MeOH (260 mL) was mixed with palladium/carbon (2 g) and subjected to hydrogenation (50 psi) for 20 h. The reaction mixture was filtered through a Celite pad, concentrated to yield 8.7 g of 2-amino-4,5-dimethoxybenzamide (100%).

2-(4-(Dimethylamino)naphthalen-1-yl)-6,7-dimethoxyquinazolin-4(3H)-one was synthesized from 2-amino-4,5-dimethoxy-benzamide and 4-Dimethylamino-naphthalene-1-carbaldehyde, using the method described for 5,7-dimethoxy-2-(pyridin-2-yl)quinazolin-4(3H)-one. 2-(4-(dimethylamino)naphthalen-1-yl)-6,7-dimethoxy-quinazolin-4(3H)-one (159 mg, 56%) was isolated as a white solid. Selected data: MS (m/z): 376.13; MP 235.5-236.5° C.

### Example 40

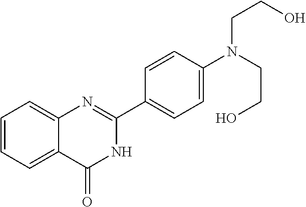 [SMILES: O=c1[nH]c(-c2ccc(N(CCO)CCO)cc2)nc2ccccc12]

2-(4-(bis(2-hydroxyethyl)amino)phenyl)quinazolin-4(3H)-one

2-(4-(Bis(2-hydroxyethyl)amino)phenyl)quinazolin-4(3H)-one was synthesized from anthranilamide and 4-[bis-(2-hydroxy-ethyl)-amino]-benzaldehyde, using the method described for 5,7-dimethoxy-2-(pyridin-2-yl)quinazolin-4(3H)-one. 2-(4-(Bis(2-hydroxyethyl)amino)phenyl)quinazolin-4(3H)-one (150 mg, 42%) was isolated as a brown solid. Selected data: MS (m/z): 326.03; MP 228-230° C.

### Example 41

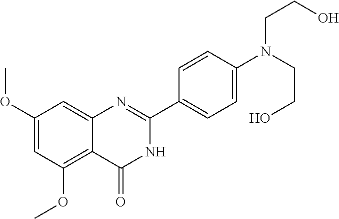 [SMILES: COc1cc(OC)c2c(=O)[nH]c(-c3ccc(N(CCO)CCO)cc3)nc2c1]

### 2-(4-(bis(2-hydroxyethyl)amino)phenyl)-5,7-dimethoxyquinazolin-4(3H)-one

2-(4-(Bis(2-hydroxyethyl)amino)phenyl)-5,7-dimethoxyquinazolin-4(3H)-one was synthesized from 2-amino-4,6-dimethoxybenzamide and 4-[bis-(2-hydroxy-ethyl)-amino]-benzaldehyde, using the method described for 5,7-dimethoxy-2-(pyridin-2-yl)quinazolin-4(3H)-one. 2-(4-(bis(2-hydroxyethyl)amino)phenyl)-5,7-dimethoxy-quinazolin-4(3H)-one (120 mg, 41%) was isolated as a yellow solid. Selected data: MS (m/z): 386.15; MP 249-251° C.

### Example 42

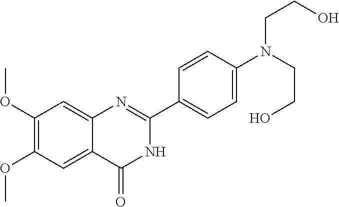 [SMILES: COc1cc2nc(-c3ccc(N(CCO)CCO)cc3)[nH]c(=O)c2cc1OC]

### 2-(4-(bis(2-hydroxyethyl)amino)phenyl)-6,7-dimethoxyquinazolin-4(3H)-one

2-(4-(Bis(2-hydroxyethyl)amino)phenyl)-6,7-dimethoxyquinazolin-4(3H)-one was synthesized from 2-amino-4,5-dimethoxy-benzamide and 4-(N,N-bis(2-hydroxyethyl)amino)benzaldehyde, using the method described for 5,7-dimethoxy-2-(pyridin-2-yl)quinazolin-4(3H)-one. 2-(4-(Bis(2-hydroxyethyl)amino)phenyl)-6,7-dimethoxyquinazolin-4(3H)-one (72 mg, 24%) was isolated as a yellow solid. Selected data: MS (m/z): 386.15; MP 268-270° C.

### Example 43

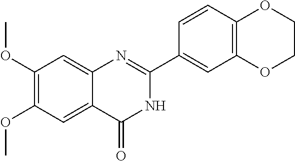 [SMILES: COc1cc2nc(-c3ccc4c(c3)OCCO4)[nH]c(=O)c2cc1OC]

### 2-(2,3-dihydrobenzo[b][1,4]dioxin-6-yl)-6,7-dimethoxyquinazolin-4(3H)-one

2-(2,3-Dihydrobenzo[b][1,4]dioxin-6-yl)-6,7-dimethoxyquinazolin-4(3H)-one was synthesized from 2-amino-4,5-dimethoxybenzamide and 2,3-dihydro-benzo[1,4]dioxine-6-carbaldehyde, using the method described for 5,7-dimethoxy-2-(pyridin-2-yl)quinazolin-4(3H)-one. 2-(2,3-Dihydrobenzo[b][1,4]dioxin-6-yl)-6,7-dimethoxy-quinazolin-4(3H)-one (180 mg, 69%) was isolated as a light yellow solid. Selected data: MS (m/z): 341.03; MP 316.4-318.2° C.

### Example 44

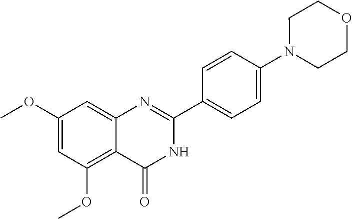 [SMILES: COc1cc(OC)c2c(=O)[nH]c(-c3ccc(N4CCOCC4)cc3)nc2c1]

### 5,7-dimethoxy-2-(4-morpholinophenyl)quinazolin-4(3H)-one

A solution of 4-iodobenzaldehyde (1 g, 4.31 mmol) in MeOH (50 mL) was mixed with trimethyl orthoformate (4 mL, 36.10 mmol) and p-toluenesulfonic acid (5 mg). The reaction was stirred at room temperature for 3 h and then quenched by adding excess of sodium bicarbonate solid and stirred for 1 h. The solid was removed by filtration and the filtrate was concentrated to yield 1-dimethoxymethyl-4-iodo-benzene (1.2 g, 100%). A mixture of 1-dimethoxymethyl-4-iodo-benzene (1.2 g, 4.31 mmol), cesium carbonate (1.4 g, 4.31 mmol), morpholine (0.375 g, 4.31 mmol), and palladium tetrakis(triphenyl)phosphine (0.25 g, 0.216 mmol) in toluene (60 mL) and tert-butanol (10 mL) was thoroughly degassed and stirred at 110° C. for 28 h. The reaction was quenched by adding water (50 mL), extracted with DCM (3×100 mL), concentrated to afford a solid residue. Purification by column chromatography left 4-(4-dimethoxymethyl-phenyl)-morpholine (0.61 g, 60%). A solution of 4-(4-dimethoxymethyl-phenyl)-morpholine (0.61 g, 2.58 mmol) in THF (20 mL) was mixed with HCl in ether (10 mL, 10 mmol) and stirred at room temperature for 2 h. The reaction mixture was then neutralized with 1 N sodium bicarbonate aqueous to pH 9 and extracted with DCM (3×100 mL), to afford 4-morpholin-4-yl-benzaldehyde (0.37 g, 75%).

A mixture of 2-amino-4,6-dimethoxybenzamide (0.15 g, 0.765 mmol), 4-morpholin-4-yl-benzaldehyde (0.15 g, 0.765 mmol), sodium hydrogensulfite (0.136 g, 0.765 mmol) and p-toluenesulfonic acid (10 mg) in N,N-dimethyl acetamide (10 mL) was stirred at 155° C. for 14 h. The reaction mixture was cooled to room temperature and diluted with water (50 mL). The solid was collected by filtration, washed with water and MeOH to yield 5,7-dimethoxy-2-(4-morpholinophenyl)quinazolin-4(3H)-one (0.109 g, 39%).

A solution of 5,7-dimethoxy-2-(4-morpholinophenyl)quinazolin-4(3H)-one (0.109 g, 0.297 mmol) in DCM (5 mL) and MeOH (5 mL) was mixed with HCl in ether (3 mL, 3 mmol), stirred for 1.5 h, concentrated. The solid formed was rinsed with Hexanes, collected by filtration and washed with hexanes and DCM to yield the hydrochloride (0.115 g, 95%) as a brown solid. Selected data: MS (m/z): 368.13; MP 217.5-219.4° C. (hydrochloride).

### Example 45

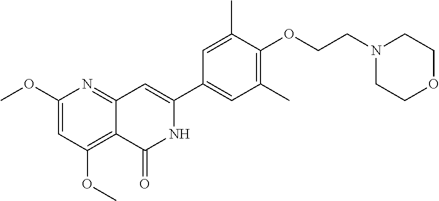 [SMILES: COc1cc(OC)c2c(=O)[nH]c(-c3cc(C)c(OCCN4CCOCC4)c(C)c3)cc2n1]

### 7-(3,5-dimethyl-4-(2-morpholinoethoxy)phenyl)-2,4-dimethoxy-1,6-naphthyridin-5(6H)-one

Malonic acid (41.62 g, 0.4 mol), 2,4,6-trichlorophenol (157.96 g, 0.8 mol) and POCl₃ (134.9 g, 80.6 mL) were mixed in a flask and stirred under reflux overnight. The reaction mixture was cooled to 70° C. and poured into ice-water. The solid was filtered off, washed with water and dried (183.73 g, quantitative). The compound from above (183.73 g, 0.4 mol), ethyl 3-aminocrotonate (51.7 g, 0.4 mol) and bromobenzene (200 mL) were mixed. The reaction mixture was heated to reflux for 4 h and then stirred at room temperature overnight, diluted with ethyl acetate and filtered off. The solid was washed with ethyl acetate to obtain a light-yellow solid (107.7 g). The solid from above (107.7 g, 0.4 mol) was dissolved in POCl₃ (300 mL, 2.5 mol) and the reaction mixture was refluxed for 2 h. POCl₃ was removed and the residue was poured into water, and extracted with DCM. The solvent was removed to obtain a crude compound (73.02 g) which was used for the next step without further purification. The compound (73.02 g, 0.31 mol) was dissolved in methanol and sodium methoxide solution in methanol (25%) was added and the mixture was refluxed overnight (˜14 h). The reaction mixture was quenched with acetic acid. DCM was added and the solvent was evaporated to leave a crude product (64.43 g), which was used for the next step without further purification. The compound (64.0 g) was dissolved in a mixture of methanol and THF. To this mixture was added lithium hydroxide (63.7 g, 1.52 mol) in water. The reaction mixture was refluxed for 3 d. The solvent was removed and conc. HCl (160 mL) was added and the mixture was concentrated. The residue was freeze dried. The crude salt (69.1 g) was used for the next step without further purification. The salt (34.6 g, 0.148 mol) was dissolved in DCM and oxalyl chloride (37.6 g, 25.8 mL) was added, followed by DMF (0.5 mL). The reaction mixture was stirred under nitrogen overnight. The solvent was evaporated in vacuo to obtain the crude acid chloride, which was used for the next step without further purification. The acid chloride was dissolved in DCM and ammonia gas was passed through the solution for 30 min. The reaction mixture was stirred overnight. Water was added and the solid was filtered off and washed with DCM. A small portion of pure A-ring building block (5 g) was isolated and crude materials (20 g) were saved.

To a solution of 4-hydroxy-3,5-dimethylbenzonitrile (5.04 g, 34.3 mmol) and PPh₃ (18.1 g, 68.6 mmol) in anhydrous THF (200 mL), were added 4-(2-hydroxyethyl)-morpholine (9.01 g, 68.6 mmol) and isopropylethylamine. To this stirred solution was added DEAD (11.95 g, 68.6 mmol) and the reaction mixture was stirred at room temperature overnight. THF was removed and ethyl acetate was added. The mixture was washed with water and brine. The crude was dissolved in DCM and washed with 1 N HCl. The aqueous layer was basified with 5% NaOH and saturated NaHCO₃ solution. The mixture was extracted with ethyl acetate and concentrated. The crude was dissolved in ether and hydrogen chloride in ether was added. The solvent was decanted off, dissolved in water, basified with solid NaHCO₃ and NaHCO₃ solution, extracted with ethyl acetate, and concentrated. The crude was purified by silica gel (100 g) column chromatography, employing 30-50% ethyl acetate in hexane as eluents to give the desired B-ring building block (0.455 g).

The A-ring building block (0.344 g, 1.75 mmol) was dissolved in anhydrous THF (50 mL) and cooled to −78° C. n-Butyllithium (3.3 mL, 5.25 mmol of 1.6 M in hexane) was added drop-wise and the temperature was increased to −20° C. for 40 min, to −10° C. for 1 h, and to −5 to −2° C. for 40 min, before the reaction mixture was cooled again to −78° C. and the B-ring building block (0.455 g, 1.75 mmol) in acetonitrile (10 mL) was added quickly. The reaction mixture was stirred at room temperature overnight (˜20 h). The dark brown solution was quenched with acetic acid and refluxed for 1 h. Water was added and extracted with DCM. The crude was purified by silica gel (50 g) column chromatography, using hexane (500 mL), hexane:ethyl acetate (1:1, 750 mL), and then hexane:ethyl acetate:methanol (3:2:1) as eluents, to give 7-(3,5-dimethyl-4-(2-morpholinoethoxy)phenyl)-2,4-dimethoxy-1,6-naphthyridin-5(6H)-one (100 mg, 13%) as an off-white solid. Selected data: MS (m/z): 440.28; MP 212.5-212.9° C.

### Example 46

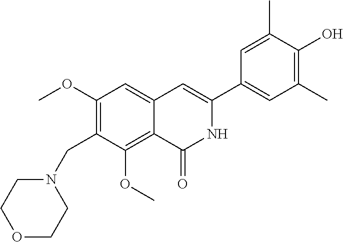 [SMILES: COc1cc2cc(-c3cc(C)c(O)c(C)c3)[nH]c(=O)c2c(OC)c1CN1CCOCC1]

### 3-(4-hydroxy-3,5-dimethyl phenyl)-6,8-dimethoxy-7-(morpholinomethyl)isoquinolin-1(2H)-one

Methyl acetoacetate (69.67 g, 0.6 mol) in dry THF (350 mL) was cooled to −5° C. and sodium hydride in mineral oil (24.5 g, 60%) was added at −5 to 0° C. over 30 min. Diketene (50.4 g) in dry THF (80 mL) was added drop-wise at 5° C. over 20 min. The resulting solution was allowed to stir for 1.0 h at −5° C., after which it was allowed to warm to room temperature and stir overnight. Acetic acid (35 mL) was added and the THF solvent was removed. Water (200 mL) and ethyl acetate (300 mL) were added to the residue and the pH was adjusted to 5.0 by addition of HCl solution. The organic layer was separated and washed with brine and dried over sodium sulfate. After column purification and recrystallization, compound A (26.6 g, 24.3%) was obtained.

Sodium hydride in mineral oil (11.2 g, 0.279 mol, 60%) was added to compound A (24.8 g, 0.136 mol) in DMF (150 mL). The reaction was cooled to −30° C. and methyl iodide (21.3 mL, 0.341 mol) was added and the reaction was kept at room temperature overnight. Sodium iodide was filtered off and DMF was removed. The residue was mixed with water (100 mL) and extracted with ethyl acetate. The organic layer was washed with brine and dried over sodium sulfate. The crude mixture was purified by column chromatography to yield compound B (11.40 g, 39.9%). To a solution of compound B (11.4 g, 0.054 mole) in dry CCl₄ (90 mL) was added N-bromosuccinimide (10.6 g, 0.0596 mol). The mixture was refluxed overnight and CCl₄ solvent was removed. Water (100 mL) was added to the residue. After stirring for a while the solid was filtered off and washed with water, ethyl acetate (10 mL) and hexane (30 mL) to yield compound (13.1 g, 83.9%). Compound C (12.5 g, 0.043 mol), chloromethyl methyl ether (81.0 g) and anhydrous zinc chloride (7.0 g, 0.051 mol) were kept at room temperature overnight. Chloromethyl methyl ether was removed and the residue was mixed with water and the pH was adjusted to 7.0 using sodium bicarbonate. The mixture was extracted with ethyl acetate. The organic layer was washed with brine and dried over sodium sulfate. Compound D (7.39 g, 50.6%) was obtained after column chromatography. A solution of compound D (7.39 g, 0.022 mol), morpholine (7.62 g, 0.088 mol) and anhydrous THF (20 mL) was kept at room temperature overnight. The solvent was evaporated. Water and ethyl acetate were added to the residue, and pH was adjusted to 9.0 with sodium bicarbonate. The organic layer was washed with brine and dried over sodium sulfate, and concentrated. Compound E (5.4 g, 63.8%) was obtained after column chromatography. The hydrogenation reaction was carried out at 50 psi with compound E (5.4 g, 0.014 mol) in THF (100 mL) and triethyl amine (3.9 mL) with 10% Pd/C (2.6 g) as a catalyst for 2 d. After the catalyst was filtered off, the organic layer was purified by column chromatography to yield product F (3.20 g, 74.4%). Compound F (3.20 g, 0.0103 mol) was dissolved in ethanol (30 mL) and potassium hydroxide (2.31 g, 0.041 mol) in water (20 mL) was added and the reaction mixture was heated to 100° C. overnight. The solvent was removed, pH was adjusted to 6.0 and the water was removed. The residue was further dried under high vacuum and the compound was extracted with ethanol to yield compound G (2.95 g, 99%). Compound G (1.80 g, 6.1 mmol) with thionyl chloride (3 mL, 0.0411 mol) was refluxed for 1 h before the excess thionyl chloride was removed and the residue was dried under high vacuum. Anhydrous THF (20 mL) was added and ammonia gas was bubbled into the reaction mixture for 2 h. THF was removed and pH was adjusted to 8.0-9.0. The mixture was extracted with dichloromethane and dried over sodium sulfate to give compound H (1.30 g, 72.4%).

NaH in mineral oil (1.14 g, 0.0285 mol, 60%) was added to 4-hydroxy-3,5-dimethylbenzonitrile (4.0 g, 0.027 mol) in anhydrous DMF (20 mL) followed by benzyl bromide (3.27 mL, 0.027 mol). The reaction was kept at room temperature overnight. The reaction mixture was poured into water and the solid was filtered off and washed with hexane to yield Compound I (5.7 g, 89%). Compound I was used for the next step reaction without further purification. BuLi (1.60 M, 10.2 mL) was added drop-wise to compound H (0.8 g, 2.72 mmol) in anhydrous THF (25 mL) at −10° C. The reaction mixture was kept at 0° C. for one h before the cooling bath was removed. The reaction mixture was stirred for 45 minutes. Compound I (0.65 g, 2.72 mmol) in anhydrous THF (5 mL) was added drop-wise at −10° C. and the reaction was continued for a further 45 min. Water (20 mL) was added. The mixture was extracted with ethyl acetate. The solvent was removed and the residue was purified by column chromatography to yield compound J (0.180 g, 12.8%). Compound J (180 mg) in methanol (80 mL) was hydrogenated at 50 psi for 3 h, using 10% Pd/C as the catalyst. The catalyst and solvent were removed and the residue was purified by column chromatography to yield 3-(4-hydroxy-3,5-dimethylphenyl)-6,8-dimethoxy-7-(morpholinomethyl)isoquinolin-1(2H)-one (28 mg, 18.8%) as a white solid. Selected data: MS (m/z): 424.21; MP 158-161° C.

### Example 47

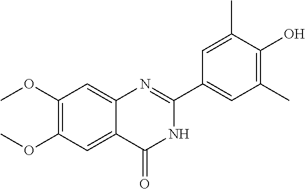

### 2-(4-hydroxy-3,5-dimethylphenyl)-6,7-dimethoxyquinazolin-4(3H)-one

To a solution of 2-amino-4,5-dimethoxybenzamide (0.157 g, 0.8 mmol) in N,N-dimethylacetamide (5 mL) were added 3,5-dimethyl-4-hydroxybenzaldehyde (0.120 g, 0.8 mmol), sodium hydrogen sulphite (58.5%, 0.156 g, 0.88 mmol) and p-toluenesulfonic acid (15 mg, 0.08 mmol). The reaction mixture was stirred at 150° C. for 3 h. The reaction mixture was cooled to room temperature and water (40 mL) was added. A white precipitate was formed and filtered off, washed with water and a small amount of methanol and dried under vacuum to give 2-(4-hydroxy-3,5-dimethylphenyl)-6,7-dimethoxyquinazolin-4(3H)-one (0.230 g, 88% yield) as an off-white solid. Selected data: MS (ES) m/z: 327.12; MP >300° C.

### Example 48

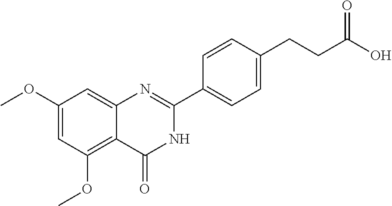 [SMILES: COc1cc(OC)c2c(=O)[nH]c(-c3ccc(CCC(=O)O)cc3)nc2c1]

### 3-(4-(5,7-dimethoxy-4-oxo-3,4-dihydroquinazolin-2-yl)phenyl)propanoic acid

To a solution of 4-iodobenzaldehyde (0.116 g, 0.5 mmol), acrolein diethylacetal (0.3 mL, 1.5 mmol), tetra-n-butylammonium chloride (0.139 g, 0.5 mmol) and triethylamine in anhydrous dimethylformamide (2 mL), palladium acetate (0.003 g, 0.015 mmol) was added. The reaction mixture was heated at 90° C. and stirred for 16 h. The reaction mixture was diluted with 2 N hydrochloric acid and extracted with diethyl ether. The solvent was evaporated in vacuo to leave a residue which was purified by column chromatography (silica gel) employing 1-5% ethyl acetate in hexane as eluents to obtain 3-(4-formyl-phenyl)-propionic acid ethyl ester (0.734 g).

To a round-bottomed flask were added 2-amino-4,6-dimethoxy-benzamide (0.161 g, 0.82 mmol), 3-(4-formyl-phenyl)-propionic acid ethyl ester (0.170 g, 0.82 mmol), sodium bisulfite (0.160 g, 0.902 mmol), p-toluenesulfonic acid (0.016 g, 0.082 mmol) and N,N-dimethylacetamide (10 mL). The reaction mixture was refluxed at 155° C. for 16 h before being cooled to room temperature. Water was added and the precipitated solid was filtered off and washed with water and methanol to obtain 3-[4-(5,7-dimethoxy-4-oxo-3,4-dihydro-quinazolin-2-yl)-phenyl]-propionic acid ethyl ester (0.304 g, 97%). The compound 3-[4-(5,7-dimethoxy-4-oxo-3,4-dihydro-quinazolin-2-yl)-phenyl]-propionic acid ethyl ester (0.304 g, 0.795 mmol) was taken up in a 1:1 mixture of THF and methanol (6 mL). A solution of potassium hydroxide (0.089 g, 1.59 mmol) in water (6 mL) was added to the reaction mixture and stirred at room temperature for 16 h. The solvent was removed and the reaction mixture was acidified with 1 N hydrochloric acid. The precipitated solid was filtered off and washed with water and methanol. The solid was further washed with methanol to obtain 3-(4-(5,7-dimethoxy-4-oxo-3,4-dihydroquinazolin-2-yl)phenyl)propanoic acid (0.143 g, 51%). Selected data: MS (ES) m/z: 355.0; MP 250.6-251.1° C.

### Example 49

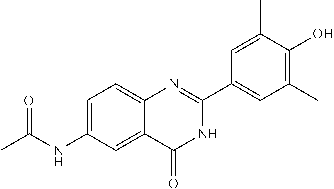 [SMILES: CC(=O)Nc1ccc2nc(-c3cc(C)c(O)c(C)c3)[nH]c(=O)c2c1]

### N-(2-(4-hydroxy-3,5-dimethylphenyl)-4-oxo-3,4-dihydroquinazolin-6-yl)acetamide

To a round-bottomed flask were added 2-amino-5-nitro-benzamide (0.681 g, 3.76 mmol), 4-hydroxy-3,5-dimethyl-benzaldehyde (0.565 g, 3.76 mmol), sodium bisulfite (0.747 g, 4.2 mmol), p-toluenesulfonic acid, monohydrate (0.072 g, 0.376 mmol) and N,N-dimethylacetamide (60 mL). The reaction mixture was refluxed at 155° C. for 16 h before being cooled to room temperature. Water was added and the precipitated solid was filtered off, washed with water and methanol to obtain a crude which was purified by column chromatography (silica gel (50 g) employing 1-20% methanol in dichloromethane as eluents, to obtain 2-(4-hydroxy-3,5-dimethyl-phenyl)-6-nitro-3H-quinazolin-4-one (0.220 g, 19%). The compound 2-(4-hydroxy-3,5-dimethyl-phenyl)-6-nitro-3H-quinazolin-4-one (0.220 g, 0.71 mmol) was hydrogenated in dimethyl formamide (20 mL) using palladium on activated carbon (0.076 g, 0.071 mmol) at room temperature for 14 h. The solvent was evaporated and the crude was purified by column chromatography (silica gel 25 g) employing 1-5% methanol in dichloromethane as eluents to obtain 6-amino-2-(4-hydroxy-3,5-dimethyl-phenyl)-3H-quinazolin-4-one (0.132 g). The compound 6-amino-2-(4-hydroxy-3,5-dimethyl-phenyl)-3H-quinazolin-4-one was dissolved in pyridine under nitrogen. Acetic anhydride was added at room temperature and stirred for 4 h. Pyridine was removed and the residue was dried. Methanol was added to the flask and a solution of potassium carbonate in water was added and stirred for 4 h. The solvent was removed, acidified with 1 N hydrochloric acid and the precipitated solid was filtered off and dried to obtain N-(2-(4-hydroxy-3,5-dimethylphenyl)-4-oxo-3,4-dihydroquinazolin-6-yl)acetamide (0.037 g, 17%). Selected data: MS (ES) m/z: 324.1; MP 336.5° C. (decomposed).

### Example 50

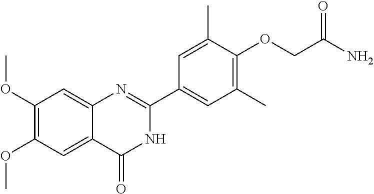 [SMILES: COc1cc2nc(-c3cc(C)c(OCC(N)=O)c(C)c3)[nH]c(=O)c2cc1OC]

### 2-(4-(6,7-dimethoxy-4-oxo-3,4-dihydroquinazolin-2-yl)-2,6-dimethylphenoxy)acetamide

A solution of 4-hydroxy-3,5-dimethoxybenzaldehyde (1.0 g, 6.66 mmol) in DMF (10 mL) was cooled to 0° C. under nitrogen. NaH (0.4 g, 10 mmol, 60% in oil) was added portion-wise. The reaction was stirred for 30 min, then 2-bromoacetamide (0.918 g, 6.66 mmol) was added and stirring was continued for 36 h at room temperature. The DMF was removed under reduced pressure and water (50 mL) was added. The mixture was extracted with EtOAc (50 mL×3). The combined organic layers were washed with an aqueous solution of NaOH (50 mL, 10%), washed with water (50 mL) and brine solution (50 mL) and dried over MgSO₄ and concentrated to give 0.6 g of crude intermediate, which was purified by flash column chromatography to provide the desired intermediate (366 mg, 26%), as a white solid. A mixture of 2-amino-4,5-dimethoxybenzamide (0.2 g, 1.019 mmol), 2-(4-formyl-2,6-dimethyl-phenoxy acetamide (0.211 g, 1.019 mmol), sodium hydrogensulfite (0.116 g, 1.121 mmol) and p-toluenesulfonic acid (20 mg) in N,N-dimethyl acetamide (5 mL) was stirred at 150° C. for 16 h under nitrogen. The reaction mixture was cooled to room temperature and water (50 mL) was added. The white precipitate was filtered off and washed with cold water (30 mL×2) and dried under high vacuum to provide 2-(4-(6,7-dimethoxy-4-oxo-3,4-dihydroquinazolin-2-yl)-2,6-dimethylphenoxy)acetamide (300 mg, 76%) as a off white solid. Selected data: MS (ES) m/z: 384.1 (M+1); MP 354-356° C.

### Example 51

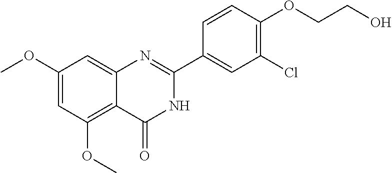 [SMILES: COc1cc(OC)c2c(=O)[nH]c(-c3ccc(OCCO)c(Cl)c3)nc2c1]

### 2-(3-chloro-4-(2-hydroxyethoxy)phenyl)-5,7-dimethoxyquinazolin-4(3H)-one

A mixture of 3-chloro-4-hydroxy-benzaldehyde (227 mg, 1.45 mmol), (2-bromoethoxy)-tert-butyldimethylsilane (347 mg, 1.45 mmol), cesium carbonate (709 mg, 2.18 mmol) and DMSO (2 mL) was stirred at 80° C. for 17 h. The reaction mixture was cooled to room temperature and water (50 mL) was added. The resulting precipitate was filtered off, washed with water, air-dried, dissolved in a small amount of ethyl acetate and purified by column chromatography. 4-[2-(tert-Butyl-dimethyl-silanyloxy)-ethoxy]-3-chloro-benzaldehyde was obtained as a white solid (yield: 267 mg, 58%). To a 100 mL round-bottomed flask was added 2-amino-4,6-dimethoxy-benzamide (166 mg, 0.85 mmol), 4-[2-(tert-Butyl-dimethyl-silanyloxy)-ethoxy]-3-chloro-benzaldehyde (267 mg, 0.85 mmol), p-toluenesulfonic acid monohydrate (21 mg, 0.11 mmol), sodium hydrogensulfite (216 mg, 1.2 mmol) and dimethylacetamide (5 mL). The mixture was stirred in a 150° C. oil bath under nitrogen for 17 h. After cooling to room temperature, water (50 mL) was added. The precipitate was filtered off, washed with water and air-dried. The crude product was purified by column chromatography to give 2-(3-chloro-4-(2-hydroxyethoxy)phenyl)-5,7-dimethoxyquinazolin-4(3H)-one (45 mg, 23%). Selected data: MS (ES) m/z: 377.03; MP 287-288° C. (decomposed).

### Example 52

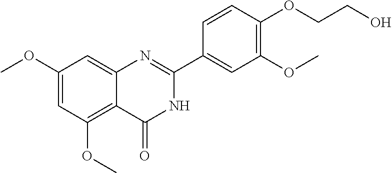 [SMILES: COc1cc(OC)c2c(=O)[nH]c(-c3ccc(OCCO)c(OC)c3)nc2c1]

### 2-(4-(2-hydroxyethoxy)-3-methoxyphenyl)-5,7-dimethoxyquinazolin-4(3H)-one

NaH (0.12 g, 0.0050 mol, 60% in mineral oil) was added to 4-hydroxy-3-methoxylbenzalde (0.636 g, 4.18 mmol) in anhydrous DMF (15 mL) and then (2-bromoethoxy)-tert-butyl-dimethylsilane (1.0 g, 4.18 mmol) was added and the reaction was kept at room temperature overnight. The reaction mixture was poured into water. The mixture was extracted with dichloromethane and the combined organic layers were passed through a column to yield 4-[2-(tert-butyl-dimethyl-silanyloxy)-ethoxy]-3-methoxybenzaldehyde (170 mg, 13%). 2-Amino-4,6-dimethoxy-benzamide (101 mg, 0.515 mmol), 4-[2-(tert-butyl-dimethyl-silanyloxy)-ethoxy]-3-methoxybenzaldehyde (160 mg, 0.515 mmol), sodium hydrogen sulfite (100 mg, 58.5%) and p-toluenesulfonic acid monohydrate (10 mg) were mixed with N,N-dimethyl acetamide (15 mL) and heated to 150° C. for 16 h. N,N-dimethyl acetamide was removed under vacuum and the residue was poured into water (50 mL). The solid was filtered off and further purified by column chromatography to yield 2-(4-(2-hydroxyethoxy)-3-methoxyphenyl)-5,7-dimethoxyquinazolin-4(3H)-one (15 mg, 7.8%). Selected data: MS (ES) m/z: 373.1; MP 246-248° C.

### Example 53

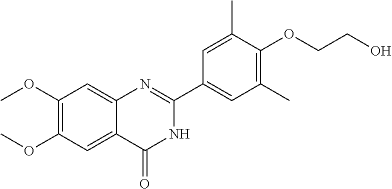 [SMILES: COc1cc2nc(-c3cc(C)c(OCCO)c(C)c3)[nH]c(=O)c2cc1OC]

### 2-(4-(2-hydroxyethoxy)-3,5-dimethylphenyl)-6,7-dimethoxyquinazolin-4(3H)-one

To a solution of 2-amino-5,6-dimethoxy-benzamide (200 mg, 1.01 mmol) and 4-[2-(tert-butyldimethylsilanoxy)ethoxy]-3,5-dimethylbenzaldehyde (314 mg, 1.01 mmol) in N,N-dimethyl acetamide (10 mL), NaHSO₃ (199 mg, 1.12 mmol) and p-TSA (19 mg, 0.1 mmol) were added and the reaction mixture was heated at 150° C. for 3 h, cooled to room temperature and poured into water. The solid was collected and washed with methanol to give 280 mg of mixture products. To a solution of the above mixture (280 mg, 0.578 mmol) in THF (20 mL), TBAF (150 mg, 0.578 mmol) was added at 0° C. and allowed to stir at room temperature for 3 h. The reaction mixture was quenched by addition of water. The organic layer was separated and the aqueous layer was extracted with ethyl acetate. The combined organic layers were washed with water, brine and dried over Na₂SO₄. The solvent was removed to give crude product. The crude product was purified by column chromatography (silica gel 230-400 mesh; 2% methanol in CH₂Cl₂ as eluent) to give 2-(4-(2-hydroxyethoxy)-3,5-dimethylphenyl)-6,7-dimethoxyquinazolin-4(3H)-one (135 mg, 63%). Selected data: MS (ES) m/z: 371.1; MP >300° C.

### Example 54

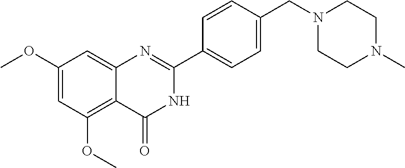

### 5,7-dimethoxy-2-(4-((4-methylpiperazin-1-yl)methyl)phenyl)quinazolin-4(3H)-one

To a solution of 4-bromomethyl-benzoic acid ethyl ester (4.0 g, 16.46 mmol) in THF (30 mL), N-methyl piperazine (3.29 g, 32.92 mmol) was added and the reaction mixture was stirred for 48 h at room temperature. Then, the reaction mixture was diluted with water and the mixture was extracted with ethyl acetate. The combined organic layers were washed well with water, brine, and dried over Na₂SO₄. The solvent was removed to give 4.0 g of crude product in 93% yield. Lithium aluminum hydride (0.771 g, 20.32 mmol) was taken in a 3-neck dry flask and THF was added on cooling. A solution of 4-(4-methyl piperazin-1-ylmethyl)-benzoic acid ethyl ester (4.0 g, 15.26 mmol) in THF (10 mL) was added slowly on cooling. After completion of addition, the reaction mixture was heated at reflux for 3 h. The reaction mixture was cooled to 0° C. and 10% NaOH solution was added, followed by water. The organic layer was separated and the aqueous layer was extracted with ethyl acetate. The combined organic layer was washed well with water, brine and dried over Na₂SO₄. The solvent was removed to give 2.4 g of crude product in 67% yield.

A 3-neck flask with anhydrous CH₂Cl₂ (100 mL) was cooled to the −78° C. Then, oxalyl chloride (1.66 g, 13.09 mmol) and DMSO (1.7 g, 21.8 mmol) were added at −78° C. and stirred for 15 min at −78° C. The solution of (4-(4-methyl piperazin-1-ylmethyl)phenyl)-methanol (2.4 g, 10.9 mmol) in CH₂Cl₂ (10 mL) was added at −78° C. and stirred at −78° C. for 1 h. Then Et₃N (4.41 g, 43.63 mmol) was added at −78° C. The reaction mixture was allowed to come to room temperature. Water was added and the organic layer was separated. The aqueous layer was extracted with CH₂Cl₂. The combined organic layer was washed with water, brine and dried over Na₂SO₄. Then, solvent was removed to give 2.23 g of crude product in 94% yield.

To a solution of 2-amino-4,6-dimethoxy-benzamide (150 mg, 0.76 mmol) and 4-(4-methyl piperazin-1-ylmethyl)benzaldehyde (166 mg, 0.76 mmol) in N,N-dimethyl acetamide (10 mL), NaHSO₃ (149 mg, 0.84 mmol) and p-TSA (319 mg, 1.68 mmol) were added and the reaction mixture was heated at 150° C. for 3 h. The mixture was cooled to room temperature and water was added and neutralized by addition of NaHCO₃. The solvent was removed under reduced pressure to give the crude product. The crude was purified by column chromatography (silica gel 230-400 mesh; 4% NH₃ in methanol/CH₂Cl₂ as eluent) to give the product 5,7-dimethoxy-2-(4-((4-methylpiperazin-1-yl)methyl)phenyl)quinazolin-4(3H)-one as a free base, which was converted to the hydrochloride salt (115 mg, 35%). Selected data: MS (ES) m/z: 395.2; MP 275-277° C. (hydrochloride).

### Example 55

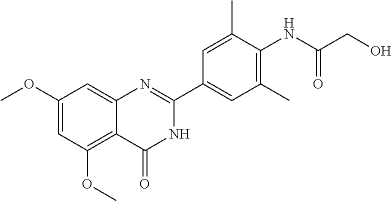 [SMILES: COc1cc(OC)c2c(=O)[nH]c(-c3cc(C)c(NC(=O)CO)c(C)c3)nc2c1]

### N-(4-(5,7-dimethoxy-4-oxo-3,4-dihydroquinazolin-2-yl)-2,6-dimethylphenyl)-2-hydroxyacetamide

A mixture of 2,6-dimethyl-phenylamine (0.62 mL, 5.0 mmol), DMSO (100 mL), conc. Aqueous HCl (36.5-38%, 5.0 mL), and dried CuCl₂ was stirred at 90° C. under nitrogen for 5 h. The reaction was quenched with water. The pH of the mixture was adjusted to −8 using a 10% sodium hydroxide solution. The mixture was extracted with ether (3×100 mL). The solution was dried over Na₂SO₄ and concentrated to dryness. The resulting brown oil was dissolved in dichloromethane (anhydrous, 20 mL) and N-ethyldiisopropylamine (DIPEA, 1.0 mL, 5.8 mmol) was added. The mixture was cooled to 0° C., acetoxyacetyl chloride (0.8 mL, 7.4 mmol) was added slowly. The mixture was stirred at room temperature under nitrogen for 17 h. The mixture was concentrated to dryness and purified by column chromatography. Acetic acid (4-formyl-2,6-dimethyl-phenylcarbamoyl)-methyl ester was obtained as yellow/beige solid (96 mg). A mixture of acetic acid (4-formyl-2,6-dimethyl-phenylcarbamoyl)-methyl ester (96 mg, 0.38 mmol), 2-amino-4,6-dimethoxy-benzamide (74 mg, 0.38 mmol), p-toluenesulfonic acid monohydrate (21 mg, 0.11 mmol), sodium hydrogensulfite (96 mg, 0.53 mmol) and dimethylacetamide (3 mL) was stirred in a 150° C. oil bath under nitrogen for 17 h. After cooling to room temperature, water (50 mL) was added. The precipitate was filtered off and washed with water. The filtrate was extracted with dichloromethane, dried over Na₂SO₄, purified by column chromatography, using (1) 5% methanol/dichloromethane, and (2) 10% methanol/dichloromethane as eluents. Acetic acid [4-(5,7-dimethoxy-4-oxo-3,4-dihydro-quinazolin-2-yl)-2,6-dimethyl-phenylcarbamoyl]-methyl ester was obtained as a beige solid (70 mg, 43%). Acetic acid [4-(5,7-dimethoxy-4-oxo-3,4-dihydro-quinazolin-2-yl)-2,6-dimethyl-phenylcarbamoyl]-methyl ester (70 mg, 0.16 mmol) was dissolved in methanol/dichloromethane (10 mL) and a solution of potassium carbonate (442 mg, 20 mmol) in water was added. The solution was stirred at room temperature for 17 h. 2 N HCl was added to adjust the reaction mixture pH to −8. The mixture was then concentrated under reduced pressure. The resulting precipitate was filtered off, washed with water, air-dried, then washed with ether and dried, leaving N-(4-(5,7-dimethoxy-4-oxo-3,4-dihydroquinazolin-2-yl)-2,6-dimethylphenyl)-2-hydroxyacetamide (30 mg, 49%) as a light brown solid. Selected data: MS (ES) m/z: 384.1; MP 190-192° C.

### Example 56

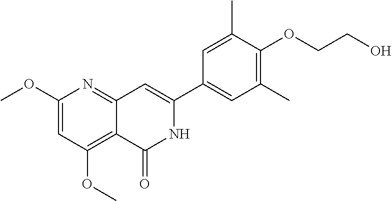 [SMILES: COc1cc(OC)c2c(=O)[nH]c(-c3cc(C)c(OCCO)c(C)c3)cc2n1]

### 7-(4-(2-hydroxyethoxy)-3,5-dimethylphenyl)-2,4-dimethoxy-1,6-naphthyridin-5(6H)-one

2-[4-(5-amino-2,4-dimethoxy-[1,6]naphthyridin-7-yl)-2,6-dimethyl-phenoxyl]-ethanol (0.302 g, 0.82 mmol) in water (5 mL) and conc. Hydrochloric acid (3 mL) were mixed with stirring. The reaction mixture was cooled to 0° C. and a solution of sodium nitrite (0.305 g, 4.42 mmol) in water (3 mL) was added dropwise. The reaction mixture was stirred at 0° C. for 40 min. To the reaction mixture was added 1 N hydrochloric acid (10 mL) and heated at 55° C. for 50 min and then stirred at room temperature overnight. The reaction mixture was extracted with dichloromethane and the aqueous layer was basified with aqueous 5% NaOH and saturated NaHCO₃ solution. Water was evaporated and the organic compound was washed with a dichloromethane/methanol solution and concentrated to leave a crude which was purified by silica gel (50 g) column chromatography, employing 50% ethyl acetate in hexane and hexane/ethyl acetate/methanol (3:2:1) as eluent, to obtain 7-(4-(2-hydroxyethoxy)-3,5-dimethylphenyl)-2,4-dimethoxy-1,6-naphthyridin-5(6H)-one (0.080 g, 26%). Selected data: MS (ES) m/z: 371.1; MP 224.9-225.4° C.

### Example 57

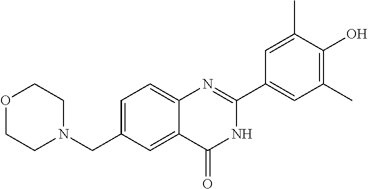 [SMILES: Cc1cc(-c2nc3ccc(CN4CCOCC4)cc3c(=O)[nH]2)cc(C)c1O]

### 2-(4-hydroxy-3,5-dimethylphenyl)-6-(morpholinomethyl)quinazolin-4(3H)-one

2-Amino-5-morpholino-4-ylmethyl-benzamide hydrochloride salt (200 mg, 0.649 mmol), 4-hydroxy-3,5-dimethylbenzalde (97.4 mg, 0.649 mmol), sodium hydrogen sulfite (127 mg, 58.5%), and p-toluenesulfonic acid monohydrate (10 mg) in N,N-dimethyl acetamide (10 mL) were heated to 150° C. for 6 h. N,N-dimethyl acetamide was removed under vacuum. The residue was poured into water (50 mL) and dichloromethane was used to extract the compound, which was further purified by column chromatography to yield 30 mg free base of 2-(4-hydroxy-3,5-dimethylphenyl)-6-(morpholinomethyl)quinazolin-4(3H)-one. The base was treated with 1.0 M HCl to give the corresponding hydrochloride (36 mg, 11.68%). Selected data: MS (ES) m/z: 366.1; mp 284-286° C. (hydrochloride).

### Example 58

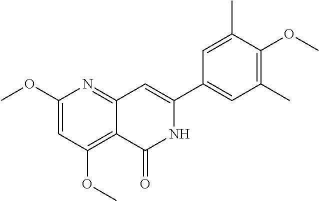

### 2,4-dimethoxy-7-(4-methoxy-3,5-dimethylphenyl)-1,6-naphthyridin-5(6H)-one

To a solution of 4,6-dimethoxy-2-methyl nicotinamide (2.0 g, 10.2 mmol) in THF (80 mL), n-butyl lithium (19.12 mL, 30.6 mmol, 1.6 M solution in hexane) was added slowly under nitrogen at −78° C. After completion of addition the mixture was stirred for 1 h at 0° C. Then cooled to −78° C. and a solution of 4-methoxy benzonitrile (1.65 g, 10.2 mmol) in THF (10 mL) was added quickly. The cooling bath was removed and the reaction mixture was allowed to warm to room temperature and stirred for 16 h at room temperature. Saturated NH₄Cl solution was added with cooling. The organic layer was washed with water, brine, dried over Na₂SO₄ and concentrated to give crude product. The crude product was purified by chromatography using 50% ethyl acetate in hexane and then 2% methanol in ethyl acetate to give 2,4-dimethoxy-7-(4-methoxy-3,5-dimethylphenyl)-1,6-naphthyridin-5(6H)-one (410 mg, 12%), as a yellow solid. Selected data: MS (ES) m/z: 341.1; mp 262-263° C. (at decomposition).

### Example 59

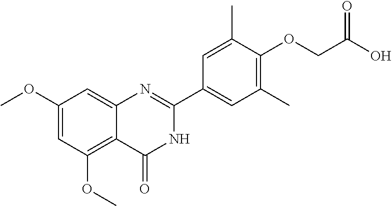 [SMILES: COc1cc(OC)c2c(=O)[nH]c(-c3cc(C)c(OCC(=O)O)c(C)c3)nc2c1]

### 2-(4-(5,7-dimethoxy-4-oxo-3,4-dihydroquinazolin-2-yl)-2,6-dimethylphenoxy)acetic acid

A solution of sodium hydroxide (2.53 g, 63.25 mmol) in water (65 mL) was added to a mixture of bromoacetic acid (5.27 g, 37.95 mmol) and 3,5-dimethyl-4-hydroxy-benzaldehyde (1.9 g, 12.65 mmol) in water (30 mL). The reaction mixture was stirred at 100° C. for 24 h. The solution was acidified to pH ˜2 with conc. HCl. The brown solid was filtered off, washed with water, dried under vacuum, and purified by column chromatography to give (4-formyl-2,6-dimethyl-phenoxy)-acetic acid as a light brown solid (0.40 g). To a solution of 2-amino-4,6-dimethoxybenzamide (0.150 g, 0.764 mmol) in N,N-dimethyl acetamide (5 mL) were added (4-formyl-2,6-dimethyl-phenoxy)-acetic acid (0.159 g, 0.764 mmol), sodium hydrogen sulphite (58.5%, 0.150 g, 0.84 mmol) and p-toluenesulfonic acid (15 mg, 0.0764 mmol). The reaction mixture was stirred at 150° C. for 3 h. it was then cooled to room temperature and water (40 mL) was added. A yellow precipitate was formed and filtered off, washed with water and a small amount of methanol. Triturated with 10% methanol in ether to give 0.084 g of compound, which was further purified by preparative HPLC to give 2-(4-(5,7-dimethoxy-4-oxo-3,4-dihydroquinazolin-2-yl)-2,6-dimethylphenoxy)acetic acid (47 mg, 13%) as a white solid. Selected data: MS (ES) m/z: 384.0; MP 270-272° C.

### Example 60

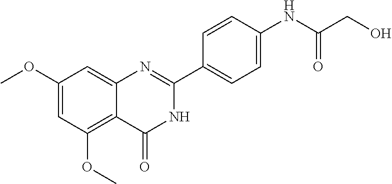 [SMILES: COc1cc(OC)c2c(=O)[nH]c(-c3ccc(NC(=O)CO)cc3)nc2c1]

### N-(4-(5,7-dimethoxy-4-oxo-3,4-dihydroquinazolin-2-yl)phenyl)-2-hydroxyacetamide

To a solution of 4-aminobenzaldehyde (1 g, 8.52 mmol) at 0° C. under nitrogen atmosphere were added triethylamine (2.3 mL, 16.5 mmol), 4-dimethylaminopyridine (0.1 g, 0.82 mmol) and acetoxyacetyl chloride (1.77 mL, 16.5 mmol). The reaction mixture was allowed to warm up to room temperature and was stirred for 2.5 h. Triethylamine (1.15 mL, 8.25 mmol) and acetoxyacetyl chloride (0.88 mL, 8.25 mmol) were added and the reaction mixture was stirred for 1 h more. The reaction mixture was poured into a 1 M hydrochloric acid solution (60 mL), then extracted with methylene chloride (20 mL×3) and the combined organic layers were washed with saturated aqueous sodium bicarbonate solution and dried over anhydrous sodium sulfate. The crude solid (3.17 g) was purified by flash column chromatography to provide pure acetic acid (4-formyl-phenylcarbamoyl)-methyl ester (1.14 g, 62% yield) as an orange solid. A mixture of 2-amino-4,6-dimethoxy-benzamide (0.15 g, 0.76 mmol), Acetic acid (4-formyl-phenylcarbamoyl)-methyl ester (0.169 g, 0.76 mmol), sodium hydrogensulfite (0.087 g, 0.84 mmol) and p-toluenesulfonic acid (15 mg, 0.076 mmol) in N,N-dimethyl acetamide (5 mL) was stirred at 150° C. for 4.5 h under nitrogen. The reaction mixture was cooled to room temperature and diluted with cold water (60 mL) to obtain a yellow solid. The yellow solid was filtered off, washed with cold water (20 mL×2), methanol and dried under vacuum to provide crude compound (230 mg, 75%).

The yellow solid was triturated with ether and methanol to provide acetic acid [4-(5,7-dimethoxy-4-oxo-3,4-dihydro-quinazolin-2-yl)-phenylcarbamoyl]-methyl ester (112 mg, 37%). To a solution of acetic acid [4-(5,7-dimethoxy-4-oxo-3,4-dihydro-quinazolin-2-yl)-phenylcarbamoyl]-methyl ester (0.23 g, 0.59 mmol) in THF/methanol mixture (3.5 mL/3.5 mL) was added potassium carbonate (0.41 g, 2.95 mmol). The reaction mixture was heated at reflux overnight and the solvent was concentrated under vacuum and diluted with water (60 mL) to obtain a precipitate. The yellow solid was filtered, washed with water (20 mL), methanol and dried under vacuum to provide crude compound. The yellow solid was triturated with ether and methanol to provide the desired compound N-(4-(5,7-dimethoxy-4-oxo-3,4-dihydroquinazolin-2-yl)phenyl)-2-hydroxyacetamide (55 mg, 55%). Selected data: MS (ES) m/z: 356.1; mp 318-319° C.

### Example 61

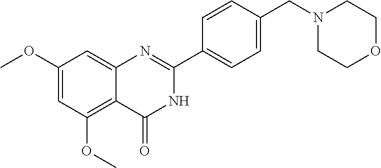 [SMILES: COc1cc(OC)c2c(=O)[nH]c(-c3ccc(CN4CCOCC4)cc3)nc2c1]

### 5,7-dimethoxy-2-(4-(morpholinomethyl)phenyl)quinazolin-4(3H)-one

To a solution of 4-bromoethyl-benzoic acid ethyl ester (4.0 g, 16.46 mmol) in THF (30 mL), morpholine (2.87 g, 32.92 mmol) was added and the reaction mixture was stirred for 48 h at room temperature. The reaction mixture was diluted with water and the product was extracted with ethyl acetate. The combined organic layers were washed with water, brine, and dried over Na₂SO₄. The solvent was removed to give 3.4 g of crude product in 83% yield.

LAH (0.571 g, 15.05 mmol) was added to a 3-neck dry flask and THF (50 mL) was added on cooling. A solution of 4-morpholin-4-ylmethyl)-benzoic acid ethyl ester (3.0 g, 12.04 mmol) in THF (10 mL) was added slowly on cooling. After completion of addition, the reaction mixture was heated at reflux for 3 h. The reaction mixture was cooled to 0° C. and a 10% NaOH solution was added carefully followed by water. The organic layer was separated and the aqueous layer was extracted with ethyl acetate. The combined organic layers were washed with water, brine and dried over Na₂SO₄. The solvent was removed to give (4-morpholin-4-ylmethyl phenyl)methanol (2.0 g, 80%). To the 3-flask anhydrous CH₂Cl₂ (100 mL) was added and cooled to −78° C. Oxalyl chloride (1.47 g, 11.59 mmol) and DMSO (1.5 g, 19.32 mmol) were added at −78° C. The reaction mixture was stirred for 15 min at −78° C. A solution of (4-morpholin-4-ylmethyl phenyl)methanol (2.0 g, 9.66 mmol) in CH₂Cl₂ (10 mL) was added at −78° C. and the mixture was stirred at −78° C. for 1 h. Then, Et₃N (3.9 g, 38.64 mmol) was added. The reaction mixture was allowed to come at room temperature. Water was added and the organic layer was isolated. The aqueous layer was extracted with CH₂Cl₂. The combined organic layers were washed with water, brine and dried over Na₂SO₄. Then solvent was removed to give crude 4-morpholin-4-ylmethyl benzaldehyde (1.6 g, 81%).

To a solution of 2-amino-4,6-dimethoxy-benzamide (150 mg, 0.76 mmol) and 4-morpholin-4-ylmethyl benzaldehyde (156 mg, 0.76 mmol) in N,N-dimethyl acetamide (10 mL), NaHSO₃ (150 mg, 0.84 mmol) and p-TSA (174 mg, 0.91 mmol) were added and the reaction mixture was heated at 150° C. for 5 h. The reaction mixture was cooled to room temperature, water was added and the mixture was neutralized with NaHCO₃. The solvent was removed under reduced pressure to give the crude product, which was purified by column chromatography to give 5,7-dimethoxy-2-(4-(morpholinomethyl)phenyl)quinazolin-4(3H)-one, which was converted to the hydrochloride salt (165 mg, 51%). Selected data: MS (ES) m/z: 382.07; MP 206-208° C. (at decomposition).

### Example 62

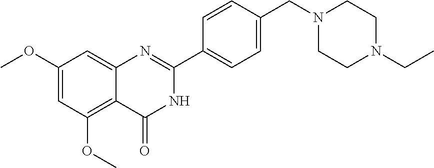 [SMILES: CCN1CCN(Cc2ccc(-c3nc4cc(OC)cc(OC)c4c(=O)[nH]3)cc2)CC1]

### 2-(4-((4-ethylpiperazin-1-yl)methyl)phenyl)-5,7-dimethoxyquinazolin-4(3H)-one

To a solution of 4-bromoethyl-benzoic acid ethyl ester (4.0 g, 16.46 mmol) in THF (30 mL), N-ethyl piperazine (3.76 g, 32.92 mmol) was added and the reaction mixture was stirred for 16 h at room temperature. The reaction mixture was diluted with water and the product was extracted with ethyl acetate. The combined organic layers were washed with water, brine, and dried over Na₂SO₄. The solvent was removed to give 4.61 g of crude 4-(4-ethyl piperazin-1-ylmethyl)-benzoic acid ethyl ester (100% yield). LAH (0.792 g, 20.86 mmol) was taken up in a 3-neck dry flask and THF (60 mL) was added on cooling. A solution of 4-(4-ethyl piperazin-1-ylmethyl)-benzoic acid ethyl ester (4.61 g, 16.69 mmol) in THF (10 mL) was added slowly on cooling. After completion of addition, the reaction mixture was heated at reflux for 2 h. The reaction mixture was cooled to 0° C., 10% NaOH solution was added, and then water was added. The organic layer was separated and the aqueous layer was extracted with ethyl acetate. The combined organic layers were washed with water, brine and dried over Na₂SO₄. The solvent was removed to give 2.78 g of crude (4-(4-ethyl piperazin-1-ylmethyl)phenyl)-methanol in 78% yield. To a 3-neck flask containing anhydrous CH₂Cl₂ (100 mL) cooled to the −78° C. oxalyl chloride (1.8 g, 14.25 mmol) and DMSO (1.85 g, 23.76 mmol) were added and the mixture was stirred for 15 min at −78° C. The solution of (4-(4-ethyl piperazin-1-ylmethyl)phenyl)-methanol (2.78 g, 11.88 mmol) in CH₂Cl₂ (10 mL) was added at −78° C. and stirred at −78° C. for 1 h. Then Et₃N (4.8 g, 47.52 mmol) was added at −78° C. The reaction mixture was allowed to come to room temperature. Water was added and the organic layer was separated. The aqueous layer was extracted with CH₂Cl₂. The combined organic layers were washed with water, brine and dried over Na₂SO₄. Then, solvent was removed to give crude 4-(4-ethyl piperazin-1-ylmethyl)benzaldehyde (2.5 g, 91%).

To a solution of 2-amino-4,6-dimethoxy-benzamide (150 mg, 0.76 mmol) and 4-(4-ethyl piperazin-1-ylmethyl)benzaldehyde (177 mg, 0.76 mmol) in N,N-dimethyl acetamide (10 mL), NaHSO₃ (150 mg, 0.84 mmol) and p-TSA (319 mg, 1.68 mmol) were added and the reaction mixture was heated at 150° C. for 5 h. The reaction mixture was cooled to room temperature, water was added and the mixture was neutralized with NaHCO₃. The solvent was removed under reduced pressure to give the crude product, which was purified by column chromatography to give 2-(4-((4-ethylpiperazin-1-yl)methyl)phenyl)-5,7-dimethoxy-quinazolin-4(3H)-one (87 mg, 27%), which was converted to the hydrochloride salt. Selected data: MS (ES) m/z: 409.11; MP 278-280° C. (at decomposition).

### Example 63

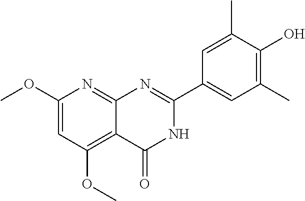

### 2-(4-hydroxy-3,5-dimethylphenyl)-5,7-dimethoxypyrido[2,3-d]pyrimidin-4(3H)-one

A mixture of dimethyl acetone-1,3-dicarboxylate (200 g, 1.15 mol), cyanamide (48.3 g, 1.15 mol), and Ni(acac)₂ (14.75 g, 0.0574 mol) in dioxane (200 mL) was heated to reflux for 16 h and then cooled to room temperature. The precipitate was filtered off, and the solid was mixed with methanol (200 mL) and stirred for 30 min and filtered again to give 93 g product (44% yield). In a 1 L flask with a reflux condenser was added the product from step one (93.0 g, 0.505 mol) and POCl₃ (425 mL) and the reaction mixture was heated to reflux for 35 min. POCl₃ (300 mL) was evaporated under vacuum. The residue was poured into ice and water (400 mL), which was neutralized with KOH to pH 6-7. The precipitate was filtered off and extracted with ethyl acetate (2×300 mL). The organic solution was concentrated and purified by column chromatography to give methyl 2-amino-4,6-dichloropyridine-3-carboxylate (22.5 g, 20.1%). In a 500 mL flask with reflux condenser was added methyl 2-amino-4,6-dichloropyridine-3-carboxylate (22.5 g, 0.101 mol) and 25 wt % sodium methoxide in methanol (88 mL, 0.407 mol), together with methanol (20 mL). The mixture was heated to reflux for 5 h then cooled to room temperature. Acetic acid (15 mL) was added to the mixture and the pH was adjusted to ˜7.0. Methanol was removed and the residue was poured into water (100 mL). The precipitated solid was filtered off and rinsed with water (3×200 mL) to give methyl 2-amino-4,6-dimethoxypyridine-3-carboxylate (18.5 g, 86.4%). In a 500 mL flask with a reflux condenser was added methyl 2-amino-4,6-dimethoxypyridine-3-carboxylate (18.5 g, 0.0872 mol), potassium hydroxide (19.5 g, 0.349 mol) in water (80 mL) and ethanol (100 mL). The mixture was heated to 80° C. for 16 h. The solvent was removed and aqueous HCl was used to adjust pH to 6.0. The water was removed by lyophilization. The obtained solid was extracted with methanol to yield 2-amino-4,6-dimethoxy-nicotinic acid in quantitative yield. 2-Amino-4,6-dimethoxy-nicotinic acid (17.2 g, 0.0872 mol) was added to THF (110 mL). 1-[3-(dimethylamino)propyl]-3-ethylcarbodiimide hydrochloride (21.73 g, 0.113 mol), 1-hydroxybenzotriazole hydrate (12.96 g, 0.0959 mol) and 4-methyl morpholine (9.7 g, 0.0959 mol) were then added to the suspension. After stirring for 10 min at room temperature, 50% v/v ammonium hydroxide (18.3 g, 0.262 mol) was added. The reaction mixture was kept at room temperature for 16 h. THF was removed and the residue was poured into cold water (100 mL). The precipitate was filtered off and further washed with cold water to yield 5.3 g of the pure desired compound. The aqueous solution was further extracted with dichloromethane (3×150 mL) to yield 8.4 g crude product, which was further purified by column chromatography to give a total of 10.8 g (62.8%) of 2-amino-4,6-dimethoxy-nicotinamide.

To a solution of 2-amino-4,6-dimethoxy-nicotinamide (1.40 g, 7.1 mmol) and 4-hydroxy-3,5-dimethylbenzaldehyde (1.07 g, 7.1 mmol) in N,N-dimethyl acetamide (20 mL), NaHSO₃ (1.39 g, 7.81 mmol) and p-TSA (0.675 g, 3.55 mmol) were added and the reaction mixture was heated at 150° C. overnight. The solvent was removed under reduced pressure. The residue was diluted with water and the solid was collected and further washed with methanol. The crude product was purified by column chromatography (silica gel 230-400 mesh; 2% methanol in CH₂Cl₂ as eluent) to give 2-(4-hydroxy-3,5-dimethylphenyl)-5,7-dimethoxypyrido[2,3-d]pyrimidin-4(3H)-one (0.92 g, 39.6%). Selected data: MS (ES) m/z: 328.07; MP 297-299° C.

### Example 64

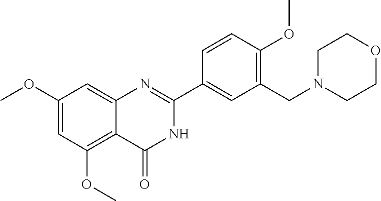 [SMILES: COc1cc(OC)c2c(=O)[nH]c(-c3ccc(OC)c(CN4CCOCC4)c3)nc2c1]

### 5,7-dimethoxy-2-(4-methoxy-3-(morpholinomethyl)phenyl)quinazolin-4(3H)-one

5,7-Dimethoxy-2-(4-methoxy-3-(morpholinomethyl)phenyl)quinazolin-4(3H)-one was synthesized from 2-amino-4,6-dimethoxybenzamide and 4-methoxy-3-morpholin-4-ylmethyl-benzaldehyde, using the method described for 5,7-dimethoxy-2-(pyridin-2-yl)quinazolin-4(3H)-one. 5,7-Dimethoxy-2-(4-methoxy-3-(morpholinomethyl)phenyl)quinazolin-4(3H)-one (65 mg, 28%) was isolated as a light yellow solid. Selected data: MS (m/z): 412.07; MP 282.7-284.5° C.

### Example 65

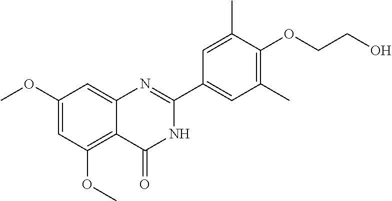 [SMILES: COc1cc(OC)c2c(=O)[nH]c(-c3cc(C)c(OCCO)c(C)c3)nc2c1]

### 2-(4-(2-hydroxyethoxy)-3,5-dimethyl phenyl)-5,7-dimethoxypyrido[2,3-d]pyrimidin-4(3H)-one

To a solution of 2-amino-4,6-dimethoxy-nicotinamide (1.07 g, 5.42 mmol) and 4-[2-(tert-butyldimethylsilanoxy)ethoxy]-3,5-dimethylbenzaldehyde (1.67 g, 5.42 mmol) in N,N-dimethyl acetamide (25 mL), NaHSO₃ (1.06 g, 5.97 mmol) and p-TSA (1.14 g, 5.97 mmol) were added and the reaction mixture was heated at 150° C. for 16 h, cooled to room temperature and poured into water. The solid was collected to give 3.25 g of crude product. To a solution of the crude product (3.25 g, 6.70 mmol) in THF (50 mL), TBAF (3.5 g, 13.4 mmol) was added at 0° C. and the mixture was stirred at room temperature for 1 h. The reaction mixture was quenched with water. The organic layer was separated and the aqueous layer was extracted with ethyl acetate. The combined organic layers were washed with water, brine and dried over Na₂SO₄. The solvent was removed, and the crude was purified by column chromatography (silica gel 230-400 mesh; 2% methanol in CH₂Cl₂ as eluent) to give 2-(4-(2-hydroxyethoxy)-3,5-dimethylphenyl)-5,7-dimethoxypyrido[2,3-d]pyrimidin-4(3H)-one (132 mg, 6%). Selected data: MS (ES) m/z: 371.99; MP 255-256° C.

### Example 66

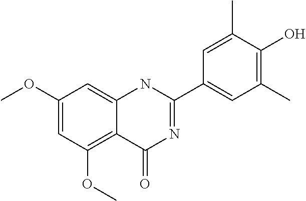 [SMILES: COc1cc(OC)c2c(=O)nc(-c3cc(C)c(O)c(C)c3)[nH]c2c1]

### 2-(4-hydroxy-3,5-dimethylphenyl)-5,7-dimethoxy-1-methylquinazolin-4(1H)-one

2-Amino-4,6-dimethoxybenzamide (0.5 g, 2.55 mmol) and methyl iodide (0.17 mL, 2.81 mmol) were mixed in a closed bomb and heated at 110° C. for 14 h. The compound was washed with a dichloromethane-methanol mixture. After removing the solvent, the crude was purified by silica gel column chromatography (40 g) employing 1-5% methanol in dichloromethane to give 2,4-dimethoxy-6-methylamino-benzamide (0.027 g, 50.4%).

The compound 3,5-dimethyl-4-hydroxybenzoic acid (5.04 g, 30.33 mmol) was mixed with pyridine (20 mL). Acetic anhydride (3.72 g, 36.4 mmol) was added and the mixture was stirred at room temperature for 4 h. The solvent was evaporated in vacuo to obtain 4-acetoxy-3,5-dimethyl-benzoic acid in quantitative yield (6.33 g). The compound 4-acetoxy-3,5-dimethyl-benzoic acid (0.36 g, 1.73 mmol)) was dissolved in dichloromethane (5 mL) and oxalyl chloride (0.3 mL, 3.46 mmol) was added dropwise, followed by 1 drop of DMF. The reaction mixture was stirred at room temperature under nitrogen for 2 h. The solvent was evaporated in vacuo to obtain acetic acid 4-chlorocarbonyl-2,6-dimethyl-phenyl ester in quantitative yield (0.392 g).

A solution of 2,4-dimethoxy-6-methylamino-benzamide (0.28 g, 1.33 mmol) in pyridine (10 mL) was added to acetic acid 4-chlorocarbonyl-2,6-dimethyl-phenyl ester (1.1 eq.) and stirred at room temperature for 14 h. The solvent was removed and the reaction mixture was acidified with 1 N HCl and extracted with ethyl acetate. The solvent was removed and the crude was purified by silica gel column chromatography (40 g) employing 1% methanol in dichloromethane to give acetic acid 4-(5,7-dimethoxy-1-methyl-4-oxo-1,4-dihydro-quinazolin-2-yl)-2,6-dimethyl-phenyl ester (0.34 g, 67%). Acetic acid 4-(5,7-dimethoxy-1-methyl-4-oxo-1,4-dihydro-quinazolin-2-yl)-2,6-dimethyl-phenyl ester (0.34 g, 0.89 mmol) was dissolved in ethanol (5 mL), 5% aqueous NaOH solution (10 mL) was added dropwise and the mixture was stirred at room temperature for 1.5 h. The compound was extracted with ethyl acetate and washed with ether to give 2-(4-hydroxy-3,5-dimethylphenyl)-5,7-dimethoxy-1-methylquinazolin-4(1H)-one (0.13 g, 43%). Selected data: MS (ES) m/z: 340.17; MP 188.5-189.1° C.

### Example 67

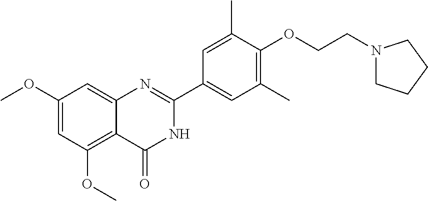 [SMILES: COc1cc(OC)c2c(=O)[nH]c(-c3cc(C)c(OCCN4CCCC4)c(C)c3)nc2c1]

### 2-(3,5-dimethyl-4-(2-(pyrrolidin-1-yl)ethoxy)phenyl)-5,7-dimethoxyquinazolin-4(3H)-one

A solution of 3,5-dimethoxy-4-hydroxybenzaldehyde (3 g, 20 mmol) and 1-(2-chloro-ethyl)-pyrrolidine hydrochloride (3.74 g, 22 mmol) in DMF (50 mL) was mixed with sodium hydride (2.24 g, 56 mmol) and potassium iodide (0.73 g, 4.4 mmol). The reaction mixture was stirred at room temperature for 2 h and then at 80° C. for an additional 2 h. The reaction was quenched with water (50 mL), extracted with EtOAc (3×100 mL), concentrated to afford an oily residue. Purification by column chromatography to yield 3.4 g of 3,5-dimethyl-4-(2-pyrrolidin-1-yl-ethoxy)-benzaldehyde (70%). A mixture of 2-amino-4,6-dimethoxy-benzamide (0.2 g, 1.02 mmol), 3,5-dimethyl-4-(2-pyrrolidin-1-yl-ethoxy)-benzaldehyde (0.251 g, 1.02 mmol), sodium hydrogensulfite (0.181 g, 1.02 mmol) and p-toluenesulfonic acid (0.234 g, 1.224 mmol) in N,N-dimethyl acetamide (10 mL) was stirred at 155° C. for 2 h. The reaction mixture was cooled to room temperature, diluted with water (50 mL), extracted with EtOAc (3×50 mL), and concentrated to afford a solid residue. The solid was further purified by column chromatography to yield about 40 mg impure product. This same reaction was repeated three times on the same scale and the impure product after each column was combined and subjected to one final column to yield 2-(3,5-dimethyl-4-(2-(pyrrolidin-1-yl)ethoxy)phenyl)-5,7-dimethoxyquinazolin-4(3H)-one (76 mg, 4%) as a light yellow solid. Selected data: MS (ES) m/z: 424.04; MP 181.0-183.2° C.

### Example 68

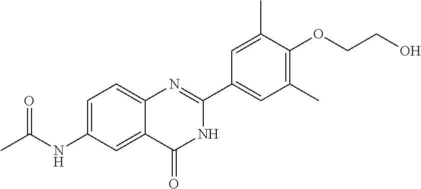 [SMILES: CC(=O)Nc1ccc2nc(-c3cc(C)c(OCCO)c(C)c3)[nH]c(=O)c2c1]

### N-(2-(4-(2-hydroxyethoxy)-3,5-dimethylphenyl)-4-oxo-3,4-dihydroquinazolin-6-yl)acetamide

To a solution of 2-amino-5-nitro-benzamide (680 mg, 3.75 mmol) and 4-[2-(tert-butyldimethylsilanoxy)ethoxy]-3,5-dimethylbenzaldehyde (1.16 g, 3.75 mmol) in N,N-dimethyl acetamide (35 mL), NaHSO₃ (736 mg, 4.14 mmol) and p-TSA (71 mg, 0.375 mmol) were added and the reaction mixture was heated at 150° C. for 5 h. The solvent was evaporated under reduced pressure. The residue was diluted with water and the solids were filtered off to give crude product (590 mg, 44%). To a solution of above crude product (490 mg, 1.38 mmol) in DMF (20 mL) and MeOH (20 mL), Pd—C (100 mg, 10%) was added and the reaction mixture was hydrogenated for 4 h at room temperature at 30 psi H₂. The reaction mixture was filtered and the solvent was evaporated to give crude product. The crude was purified by column chromatography (silica gel 230-400 mesh; 4% methanol in CH₂Cl₂ as eluent) to give 6-amino-2-(4-(2-hydroxy ethoxy)-3,5-dimethyl phenyl)-3H-quinazolin-4-one (190 mg, 42% yield). To a solution of 6-amino-2-(4-(2-hydroxy ethoxy)-3,5-dimethyl phenyl)-3H-quinazolin-4-one (95 mg, 0.29 mmol) in pyridine (5 mL), acetic anhydride (108 mg, 0.73 mmol) was added and the mixture was stirred for 16 h at room temperature. The solvent was removed and the solids were dissolved in a mixture of MeOH (10 mL) and THF (10 mL) (compound was partially soluble). Then K₂CO₃ (100 mg, 0.73 mmol) was added and the reaction mixture was stirred for 3 h at room temperature. The solvent was removed and the crude was purified by column chromatography (silica gel 230-400 mesh; 5% methanol in CH₂Cl₂ as eluent) to give N-(2-(4-(2-hydroxyethoxy)-3,5-dimethylphenyl)-4-oxo-3,4-dihydroquinazolin-6-yl)acetamide (65 mg, 60%). Selected data: MS (ES) m/z: 368.09; MP >300° C.

### Example 69

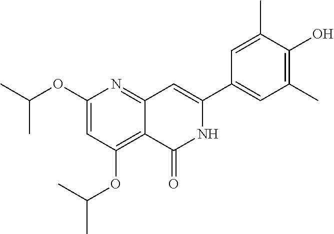 [SMILES: Cc1cc(-c2cc3nc(OC(C)C)cc(OC(C)C)c3c(=O)[nH]2)cc(C)c1O]

### 7-(4-hydroxy-3,5-dimethylphenyl)-2,4-diisopropoxy-1,6-naphthyridin-5(6H)-one

Malonic acid (5.27 g, 51 mmol), 2,4,6-trichlorophenol (20 g, 100 mmol) and phosphorus oxychloride (17.17 g, 112 mmol) were stirred under nitrogen atmosphere at reflux for 12 h. The reaction mixture was cooled to 70° C. and poured into ice water. The formed precipitate was collected, washed with water and dried under vacuum to provide the desired malonic acid bis-(2,4,6-trichloro-phenyl) ester as a white solid (23.37 g, quantitative yield). To a mixture of malonic acid bis-(2,4,6-trichloro-phenyl) ester (23.37 g, 50.5 mmol) and ethyl-3-aminocrotonate (6.38 mL, 50.5 mmol) under nitrogen atmosphere was added bromobenzene (5 mL). The reaction mixture was heated under reflux for 2.5 h then cooled to room temperature and diluted with ethyl acetate. The formed precipitate was filtered off, washed several times with ethyl acetate and dried under vacuum to afford the desired 4,6-dihydroxy-2-methyl-nicotinic acid ethyl ester as a yellow solid (13.04 g, quantitative yield). To a mixture of 4,6-dihydroxy-2-methyl-nicotinic acid ethyl ester (12.93 g, 65.57 mmol) in N,N-dimethylformamide (550 mL) and potassium carbonate (27.18 g, 196.71 mmol) under nitrogen atmosphere was added dropwise isopropyl iodide (19.65 mL, 196.71 mmol). The resulting slurry was vigorously stirred at room temperature overnight and then filtered to remove insoluble salts. The filtrate was diluted with water (300 mL) and extracted with ethyl acetate (4×400 mL). The combined organic layers were washed with brine, dried over sodium sulfate and evaporated to afford the desired 4,6-diisopropoxy-2-methyl-nicotinic acid ethyl ester as an oil which solidified on standing (15.24 g, 82.6%). To a solution of 4,6-diisopropoxy-2-methyl-nicotinic acid ethyl ester (15.24 g, 54.2 mmol) in methanol (70 mL) was added sodium hydroxide in water (70 mL). The reaction mixture was heated under reflux for 48 h. The solvent was removed under reduced pressure and concentrated hydrochloric acid was added (20 mL). The solvent was evaporated to provide the desired 4,6-diisopropoxy-2-methyl-nicotinic acid as a white salt (26.91 g, theoretical mass: 13.73 g). To a solution of 4,6-diisopropoxy-2-methyl-nicotinic acid salt (13.73 g, 54.2 mmol) in methylene chloride (160 mL) under nitrogen atmosphere was added oxalyl chloride (9.46 mL, 108.4 mmol) followed by N,N-dimethylformamide (1 mL). The reaction mixture was stirred overnight then the solvent was evaporated to obtain the desired crude acid chloride, which was used for the next step without further purification. To 50% v/v ammonia hydroxide (500 mL) at room temperature was added dropwise a solution of the crude 4,6-diisopropoxy-2-methyl-nicotinoyl chloride in methylene chloride (400 mL). The reaction mixture was stirred for 3.5 h. The solution was separated and the aqueous layer was extracted with methylene chloride (100 mL×8). The combined organic layers were dried over sodium sulfate and evaporated to afford a crude solid (6.94 g). The crude was purified by flash column chromatography to provide pure 4,6-diisopropoxy-2-methyl-nicotinamide as an orange solid (3.0 g, 21.9%). To a solution of 4,6-diisopropoxy-2-methyl-nicotinamide (0.3 g, 1.18 mmol) in THF (5 mL) under nitrogen was added 1.6 M n-BuLi solution in hexanes (3 mL, 4.75 mmol) at −20° C. The reaction mixture was allowed to warm-up to room temperature and left to stir for 2 h. The reaction was then cooled to −20° C. and a solution of 4-benzyloxy-3,5-dimethyl-benzonitrile in THF (5 mL) was added dropwise. The reaction mixture was allowed to warm to room temperature and was left to stir for 20 h. Water and acetic acid were added until pH ˜0.5. The solution was heated to 55° C. for 3 h then cooled to room temperature, diluted with ethyl acetate, separated and the aqueous layer was extracted with ethyl acetate. The combined organic layers were washed with brine, dried over sodium sulfate and evaporated under reduced pressure to provide crude orange oil (1.02 g). The crude was purified by flash column chromatography to provide pure 7-(4-benzyloxy-3,5-dimethyl-phenyl)-2,4-diisopropoxy-6H-[1,6]naphthyridin-5-one as a yellow solid (0.10 g, 17.9%). To a solution of 7-(4-benzyloxy-3,5-dimethyl-phenyl)-2,4-diisopropoxy-[1,6]naphthyridin-5-ylamine (0.10 g, 0.21 mmol) in methanol (4 mL) was added palladium on charcoal catalyst (0.06 g, 0.54 mmol). The reaction mixture was stirred under 1 atmosphere pressure of hydrogen for 20 h and diluted with methanol and filtered through a Celite pad. The solvent was evaporated under reduced pressure to provide a crude solid (0.077 g) which was triturated with ether followed by methanol to afford the desired compound 7-(4-hydroxy-3,5-dimethylphenyl)-2,4-diisopropoxy-1,6-naphthyridin-5(6H)-one (35 mg, 43.2%). Selected data: MS (ES) m/z: 383.08; MP 206-208° C.

### Example 70

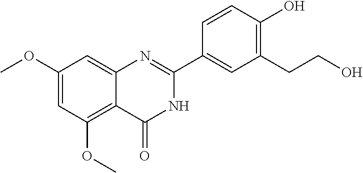 [SMILES: COc1cc(OC)c2c(=O)[nH]c(-c3ccc(O)c(CCO)c3)nc2c1]

### 2-(4-hydroxy-3-(2-hydroxyethyl)phenyl)-5,7-dimethoxyquinazolin-4(3H)-one

To a solution of 3-bromo-4-hydroxybenzaldehyde (5 g, 2.44 mmol) in acetone (100 mL) under nitrogen atmosphere was added potassium carbonate (50 6 g, 36.6 mmol). The slurry mixture was cooled to 0° C. and chloromethyl ether (9.25 mL, 12.2 mmol) was added dropwise. The ice bath was removed and the mixture was heated at 70° C. for 2.5 h. After cooling to room temperature, excess potassium carbonate was filtered off and the acetone evaporated under reduced pressure. The residue was dissolved in ethyl acetate (300 mL) and water (100 mL) was added. The organic layer was separated, washed with 0.5 N sodium hydroxide solution (100 mL×2) followed by brine and dried over sodium sulfate and concentrated to give a crude oil (6.69 g), which was purified by Flash Column Chromatography on 230-400 mesh silica gel (40-63 μm particle size) eluted with EtOAc/hexane: 2/3 to provide pure 3-bromo-4-methoxymethoxy-benzaldehyde, as an oil (4.46 g, 73.2%). To a solution of 3-bromo-4-methoxymethoxy-benzaldehyde (4.4 g, 17.9 mmol) and vinyltributyl tin (5.8 mL, 19.7 mmol) in toluene (130 mL) under nitrogen atmosphere was added an catalytic amount of tetrakis(triphenylphosphine) palladium (0.79 mg, 0.68 mmol). The resulting mixture was heated at 100° C. overnight, cooled to room temperature and a saturated potassium fluoride solution (30 mL) was added. The solution was stirred for 30 min then diluted with ethyl acetate, separated, and the aqueous layer was extracted with ethyl acetate. The combined organic layers were washed with brine, dried over sodium sulfate and evaporated under reduced pressure to provide crude yellow oil (4.6 g). The crude was purified by flash column chromatography to give pure 4-methoxymethoxy-3-vinyl-benzaldehyde as a yellow oil (1.95 g, 56.5%). To a solution of 4-methoxymethoxy-3-vinyl-benzaldehyde (1.8 g, 9.46 mmol) in THF (25 mL) under nitrogen was added borane dimethyl sulfide complex at 0° C. The solution was allowed to warm to room temperature and was stirred for 18 h. The reaction mixture was quenched at 0° C. with methanol (12 mL), hydrogen peroxide solution (8 mL) and 4 N sodium hydroxide solution (12 mL). The mixture was vigorously stirred at room temperature for 12 h and was diluted with ethyl acetate. The aqueous layer was extracted with ethyl acetate. The combined organic layers were washed with brine, dried over sodium sulfate and evaporated under reduced pressure to provide crude oil (3.2 g). The crude was purified by flash column chromatography to give pure 2-(5-hydroxymethyl-2-methoxymethoxy-phenyl)-ethanol (1.19 g, 59.5%). The mixture of 2-(5-hydroxymethyl-2-methoxymethoxy-phenyl)-ethanol (0.78 g, 3.69 mmol) and magnesium dioxide (0.086 g, 0.99 mmol) in chloroform (12 mL) was heated at 80° C. for 3 h under nitrogen. The reaction mixture was cooled to room temperature and was diluted with chloroform and filtered through a Celite pad to give the desired 3-(2-hydroxy-ethyl)-4-methoxymethoxy-benzaldehyde (0.63 g, 81.3%), which was used without further purification.

A mixture of 2-amino-4,6-dimethoxy-benzamide (0.25 g, 1.27 mmol), 3-(2-hydroxy-ethyl)-4-methoxymethoxy-benzaldehyde (0.268 g, 1.27 mmol), sodium hydrogensulfite (0.146 g, 1.4 mmol) and p-toluenesulfonic acid (0.025 g, 0.127 mmol) in N,N-dimethyl acetamide (8 mL) was stirred at 150° C. overnight under nitrogen atmosphere. The reaction mixture was cooled to room temperature, the solvent evaporated under reduced pressure. Water (70 mL) was added to obtain a solid. The yellow solid was filtered off, washed with water and dried under vacuum to provide crude 2-[3-(2-hydroxy-ethyl)-4-methoxymethoxy-phenyl]-5,7-dimethoxy-3H-quinazolin-4-one (0.182 g, 36.7%) which was used as such in the next step. A solution of 2-[3-(2-hydroxy-ethyl)-4-methoxymethoxy-phenyl]-5,7-dimethoxy-3H-quinazolin-4-one (0.18 g), 50% acetic acid solution (4 mL) and catalytic amount of concentrated sulfuric acid (0.02 mL) was heated at 70° C. for 2.5 h. After cooling to room temperature the reaction mixture was diluted with water (30 mL) to obtain a solid. The solid was filtered off, washed with water and dried under high vacuum to provide crude solid (0.135 g, 85%). The crude was purified by flash column chromatography to give pure 2-(4-hydroxy-3-(2-hydroxyethyl)phenyl)-5,7-dimethoxyquinazolin-4(3H)-one (0.035 g. 8% over 2 steps). Selected data: MS (ES) m/z: 343.0; MP 249-250.3° C.

### Example 71

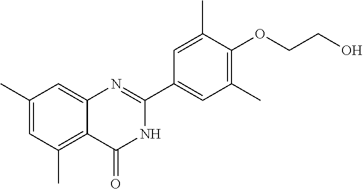 [SMILES: Cc1cc(C)c2c(=O)[nH]c(-c3cc(C)c(OCCO)c(C)c3)nc2c1]

### 2-(4-(2-hydroxyethoxy)-3,5-dimethylphenyl)-5,7-dimethylquinazolin-4(3H)-one

To a solution of 4,6-dimethyl-2-nitroaniline (3 g, 18.07 mmol) in acetic acid (20 mL) and 6 N HCl (60 mL) at 0° C. was added a solution of sodium nitrite (2.18 g, 31.62 mmol) in water (5 mL). The reaction mixture was stirred at 0° C. for 30 min after completion of addition and copper (I) cyanide (3.24 g, 3 mmol) was added pinch by pinch. The resulting mixture was stirred at 0° C. for 5 h and at room temperature for an additional 2 h. The mixture was passed through a Celite pad, extracted with EtOAc (3×100 mL), and concentrated using a rotary evaporator to afford a solid residue. The solid was further purified by column (SiO₂, hexanes/EtOAc=7:1) to yield 2-chloro-1,5-dimethyl-3-nitro-benzene (2.6 g, 81%) as a light yellow solid. A solution of 2-chloro-1,5-dimethyl-3-nitro-benzene (2.6 g, 15.7 mmol) and copper (I) cyanide (7.05 g, 78.3 mmol) in DMAC (20 mL) was stirred at reflux for 14 h. The reaction mixture was cooled to room temperature, quenched by adding water (30 mL), filtered through a Celite pad, extracted with EtOAc (3×100 mL), and concentrated using a rotary evaporator to afford a solid residue. The solid was further purified by column (SiO₂, hexanes/EtOAc=6:1) to yield 0.64 g of 2,4-dimethyl-6-nitro-benzonitrile (23%). A solution of 2,4-dimethyl-6-nitro-benzonitrile (1.1 g, 6.24 mmol) in MeOH (20 mL) and water (10 mL) was mixed with hydrogen peroxide (10 mL), DMSO (10 mL) and potassium hydroxide (0.636 g, 11.36 mmol). The reaction mixture was stirred at 60° C. for 3 h, diluted with water (100 mL), extracted with EtOAc (3×100 mL), and concentrated using a rotary evaporator to afford 4,6-dimethyl-2-nitrobenzamide (0.52 g, 43%). A solution of 4,6-dimethyl-2-nitrobenzamide (0.52 g, 2.68 mmol) in MeOH (30 mL) was mixed with palladium carbon (0.25 g). The resulting suspension was stirred at room temperature under hydrogen for 14 h. The mixture was passed through a Celite pad, concentrated using a rotary evaporator to afford 2-amino-4,6-dimethyl benzamide (0.42 g, 95%).

A mixture of 2-amino-4,6-dimethyl benzamide (0.2 g, 1.22 mmol), 4-[2-(tert-butyl-dimethyl-silanyloxy)-ethoxy]-3,5-dimethyl-benzaldehyde (0.376 g, 1.22 mmol), sodium hydrogensulfite (0.22 g, 1.22 mmol) and p-toluenesulfonic acid (0.116 g, 0.61 mmol) in N,N-dimethyl acetamide (10 mL) was stirred at 155° C. for 14 h. The reaction mixture was cooled to room temperature and diluted with water (50 mL). The solid crashed out and was collected by filtration to afford impure product. The solid was re-dissolved in THF (30 mL) and mixed with TBAF in THF (5 mL, 5 mmol). The reaction mixture was stirred at room temperature for 14 h and concentrated using a rotary evaporator to afford an oily residue. Further purification by column (SiO₂, EtOAc/DCM/MeOH=12:4:1) yielded an off-white solid. This solid was diluted with MeOH (10 mL) to make a slurry. The solid was collected by filtration and washed with MeOH to afford 2-(4-(2-hydroxyethoxy)-3,5-dimethylphenyl)-5,7-dimethylquinazolin-4(3H)-one (98 mg, 24%) as a white solid. Selected data: MS (ES) m/z: 339.10; MP 259.6-261.2° C.

### Example 72

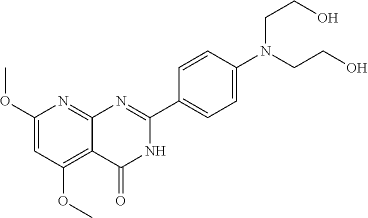 [SMILES: COc1cc(OC)c2c(=O)[nH]c(-c3ccc(N(CCO)CCO)cc3)nc2n1]

### 2-(4-(bis(2-hydroxyethyl)amino)phenyl)-5,7-dimethoxy-pyrido[2,3-d]pyrimidin-4(3H)one

To a solution of 2-amino-4,6-dimethoxy-nicotinamide (300 mg, 1.52 mmol) and 4-(bis-(2-hydroxyethyl)amino)-benzaldehyde (318 mg, 1.52 mmol) in N,N-dimethylacetamide (10 mL) were added NaHSO₃ (297 mg, 1.67 mmol) and p-TSA (376 mg, 1.98 mmol) and the reaction mixture was heated at 150° C. for 4 h, cooled to room temperature, and concentrated under reduced pressure. The residue was diluted with water and the solid was filtered off to give the crude product. The crude product was purified by column chromatography to give 2-(4-(bis(2-hydroxyethyl)amino)phenyl)-5,7-dimethoxypyrido[2,3-d]pyrimidin-4(3H)-one (60 mg, 10%). Selected data: MS (ES) m/z: 387.05; MP 277-279° C.

### Example 73

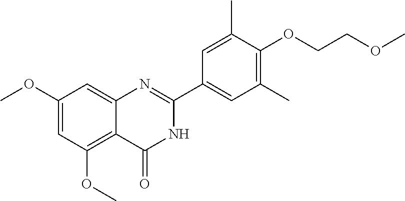

### 5,7-dimethoxy-2-(4-(2-methoxyethoxy)-3,5-dimethylphenyl)quinazolin-4(3H)-one

To a solution of 3,5-dimethyl-4-hydroxy benzaldehyde (2.0 g, 13.33 mmol) in DMF was added NaH (640 mg, 16.0 mmol, 60% in oil) and the mixture was stirred for 1 h at room temperature. A solution of 1-bromo-2-methoxy ethane (1.85 g, 13.33 mmol) was added and the mixture was stirred for 72 h at room temperature. The reaction mixture was quenched by addition of saturated NH₄Cl solution and diluted with water. The product was extracted with ethyl acetate. The combined organic layers were washed with water, brine and dried over Na₂SO₄. Upon removal of solvent, it gave 2.1 g of 4-(2-methoxy ethoxy)-3,5-dimethyl benzaldehyde (76 yield). To a solution of 2-amino-4,6-dimethoxy-benzamide (200 mg, 1.02 mmol) and 4-(2-methoxy ethoxy)-3,5-dimethyl benzaldehyde (212 mg, 1.02 mmol) in N,N-dimethyl acetamide (10 mL), NaHSO₃ (199 mg, 1.12 mmol) and p-TSA (22 mg, 0.102 mmol) were added and the reaction mixture was heated at 150° C. for 3 h. Cooled to room temperature and the solvent was evaporated under reduced pressure. The residue was diluted with water and the solid was collected to give the crude product. The crude product was purified by chromatography using 2% MeOH in CH₂Cl₂ to give 5,7-dimethoxy-2-(4-(2-methoxyethoxy)-3,5-dimethylphenyl)quinazolin-4(3H)-one (170 mg, 43%). Selected data: MS (ES) m/z: 385.10; MP 201-202° C.

### Example 74

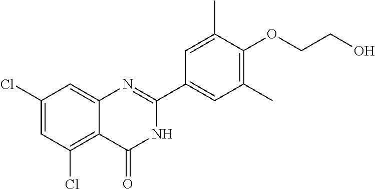 [SMILES: Cc1cc(-c2nc3cc(Cl)cc(Cl)c3c(=O)[nH]2)cc(C)c1OCCO]

### 5,7-dichloro-2-(4-(2-hydroxyethoxy)-3,5-dimethylphenyl)quinazolin-4(3H)-one

To a solution of 2-amino-4,6-dichloro-benzoic acid (0.5 g, 2.43 mmol) in THF (22 mL) under nitrogen atmosphere was added successively N-(3-Dimethylaminopropyl)-N′-ethylcarbodiimide hydrochloride (0.51 g, 2.67 mmol), N-hydroxybenzotriazole (0.36 g, 2.67 mmol) and N-methylmorpholine (0.3 mL, 2.67 mmol). The mixture was stirred for 1.5 h before a 50% ammonium hydroxide solution (1.03 mL, 14.58 mmol) was added. The mixture was stirred overnight. The solvent was evaporated under reduced pressure, water (20 mL) was added and the solution was extracted with EtOAc (50 mL×2). The combined organic layers were washed with water, brine, dried over sodium sulfate and evaporated under reduced pressure to provide crude yellow solid (0.45 g). The crude product was triturated with ether to give pure 2-amino-4,6-dichloro-benzamide (0.41 g, 82%). A mixture of 2-amino-4,6-dichloro-benzamide (0.2 g, 0.97 mmol), 4-[2-(tert-butyl-dimethyl-silanyloxy)-ethoxy]-3,5-dimethyl-benzaldehyde (0.3 g, 0.97 mmol), sodium hydrogensulfite (0.11 g, 1.05 mmol) and p-toluenesulfonic acid (0.093 g, 0.48 mmol) in N,N-dimethyl acetamide (8 mL) was stirred at 150° C. overnight under nitrogen atmosphere. The reaction mixture was cooled to room temperature, the solvent was evaporated under reduced pressure, then water (70 mL) was added and the precipitate was collected, and washed with water, dried under vacuum and triturated with ether to provide the crude mixture of 2-{4-[2-(tert-butyl-dimethyl-silanyloxy)-ethoxy]-3,5-dimethyl-phenyl}-5,7-dichloro-3H-quinazolin-4-one and 5,7-dichloro-2-[4-(2-hydroxy-ethoxy)-3,5-dimethyl-phenyl]-3H-quinazolin-4-one (0.298 g), which was used as such in the next step. To the above described mixture (0.298 g, 0.59 mmol) in tetrahydrofurane (5 mL) was added tetrabutylammonium fluoride (2.35 mL, 2.35 mmol) under nitrogen atmosphere. The reaction mixture was stirred overnight before the solvent was evaporated under reduced pressure and water was added to obtain a precipitate. The solid was filtered off, washed with water, dried under vacuum and triturated with ether to provide crude yellow solid (0.226 g, 98%). The crude was purified twice by flash column chromatography to give pure 5,7-dichloro-2-(4-(2-hydroxyethoxy)-3,5-dimethylphenyl)quinazolin-4(3H)-one (0.069 g, 19%). Selected data: MS (ES) m/z: 378.92, 380.88, 382.89; MP 260.8-262.6° C.

### Example 75

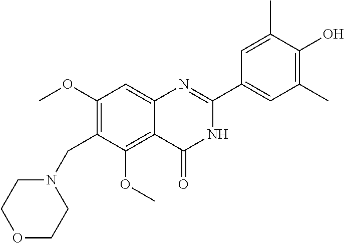 [SMILES: COc1cc2nc(-c3cc(C)c(O)c(C)c3)[nH]c(=O)c2c(OC)c1CN1CCOCC1]

### 2-(4-hydroxy-3,5-dimethylphenyl)-5,7-dimethoxy-6-(morpholinomethyl)quinazolin-4(3H)-one

To a solution of 2,6-dimethoxytoluene (50 g, 328.5 mmol) in ether (450 mL) was added freshly prepared dioxane dibromide in ether over 0.5 h. The mixture was stirred at room temperature for an additional 1.5 h and poured into a beaker containing water (500 mL). The aqueous layer was discarded and the ether layer was washed sequentially with water (2×500 mL), sodium bicarbonate (saturated aqueous) (2×500 mL), dried over sodium sulfate, and concentrated using a rotary evaporator to afford 76 g of 3-bromo-2,6-dimethoxytoluene as a colorless oil (100%). A cooling well was used to collect 300 mL of ammonia at −78° C., which was mixed with potassium (0.5 g) and ferric nitrate (0.5 g). Additional potassium (14.2 g, 364 mmol) was added at −78° C. portion-wise. The solution was stirred at −78° C. for 15 min. To this solution was slowly added 3-bromo-2,6-dimethoxytoluene (42 g, 182 mmol) in THF (100 mL). The resulting mixture was stirred at −78° C. for 3 h and then 0° C. for 1 h. The reaction was quenched by adding water (150 mL) and extracted with DCM (3×200 mL) to get a brown oil as the crude product. It was further purified by column chromatography to yield 22.1 g of 3,5-dimethoxy-4-methylaniline (73%). A solution of 3,5-dimethoxy-4-methylaniline (22.1 g, 132.3 mmol) in dioxane (380 mL) and water (380 mL) was mixed with potassium carbonate (45.6 g, 330.8 mmol) and (Boc)₂O (34.6 g 158.8 mmol) and stirred at room temperature for 14 h. The reaction mixture was then extracted with DCM (3×100 mL) and concentrated using a rotary evaporator. The resulting solid residue was purified by column chromatography. A mixture of DCM-hexanes (20 mL-300 mL) was used to make a slurry and the solid was collected by filtration and washed with hexanes to provide 28.6 g of (3,5-dimethoxy-4-methyl-phenyl)-carbamic acid tert-butyl ester (81%). A solution of (3,5-dimethoxy-4-methyl-phenyl)-carbamic acid tert-butyl ester (28.6 g, 107.1 mmol) in carbon tetrachloride (450 mL) was mixed with NBS (19.05 g, 107.1 mmol) and AIBN (1.55 g, 9.37 mmol) and the mixture was stirred at 80° C. with the light on for 2 h. The reaction was quenched by adding water (150 mL) and extracted with DCM (3×100 mL), and concentrated to afford a solid residue. Further purification by column chromatography yielded 34.9 g of (2-bromo-3,5-dimethoxy-4-methyl-phenyl)-carbamic acid tert-butyl ester (94%). A solution of (2-bromo-3,5-dimethoxy-4-methyl-phenyl)-carbamic acid tert-butyl ester (34.9 g, 100.9 mmol) in carbon tetrachloride (450 mL) was mixed with NBS (21.5 g, 121.0 mmol) and AIBN (1.55 g, 9.37 mmol) and was stirred at 80° C. with the light on for 4 h. The reaction was then quenched by adding water (150 mL) and extracted with DCM (3×100 mL), and concentrated to afford a solid residue. Further purification by column chromatography yielded 39 g of (2-bromo-4-bromomethyl-3,5-dimethoxy-phenyl)-carbamic acid tert-butyl ester (91%). A solution of (2-bromo-4-bromomethyl-3,5-dimethoxy-phenyl)-carbamic acid tert-butyl ester (39 g, 91.8 mmol) in THF (600 mL) was mixed with morpholine (45 mL, 515.0 mmol) and stirred at room temperature for 7 h. The reaction was diluted with water (300 mL), extracted with DCM (3×200 mL), and concentrated using a rotary evaporator. The residue was further purified by column (SiO₂, DCM/MeOH=20:1) to provide 35 g of (2-bromo-3,5-dimethoxy-4-morpholin-4-ylmethyl-phenyl)-carbamic acid tert-butyl ester (88%). A solution of (2-bromo-3,5-dimethoxy-4-morpholin-4-ylmethyl-phenyl)-carbamic acid tert-butyl ester (3 g, 6.94 mmol) in THF (150 mL) was mixed with NaH (0.333 g, 8.33 mmol) and stirred at room temperature for 1.5 h. The resulting mixture was cooled to −78° C. and mixed with nBuLi (3.33 mL, 8.33 mmol). The reaction was stirred for 1.5 h at −78° C. before addition of t-BuLi (8.16 mL, 13.88 mmol). The reaction was stirred at −78° C. for 1 h and carbon dioxide gas was then bubbled through for 8 h allowing the temperature to rise gradually to room temperature. The reaction was quenched by adding water (0.5 mL, 27.8 mmol) and concentrated using a rotary evaporator. The solid residue was made into slurry in minimal amount of MeOH and the solid was filtered off. The filtrate was then concentrated using a rotary evaporator and the solid was made into a slurry again in MeOH and filtered. After repeating two to three times, the filtrate was concentrated to yield 1.1 g of impure 6-tert-butoxycarbonylamino-2,4-dimethoxy-3-morpholin-4-ylmethyl-benzoic acid (40% crude yield).

A solution of 6-tert-butoxycarbonylamino-2,4-dimethoxy-3-morpholin-4-ylmethyl-benzoic acid (1.8 g, 4.54 mmol), EDCl.HCl (1.31 g, 6.82 mmol), HOBt (1.23 g, 9.09 mmol), and triethylamine (3.3 mL, 23.7 mmol) in THF (50 mL) was stirred at room temperature for 1 h. Ammonium hydroxide (50% aqueous, 10 mL) was then added to the reaction mixture. The resulting mixture was stirred at room temperature for 6 h. The reaction was quenched by adding water (50 mL), extracted with DCM (3×100 mL), and concentrated using a rotary evaporator. The residue was further purified by column (SiO₂, DCM/MeOH/EtOAc=2:1:4) to provide 0.9 g of (2-carbamoyl-3,5-dimethoxy-4-morpholin-4-ylmethyl-phenyl)-carbamic acid tert-butyl ester (50%). A solution of (2-carbamoyl-3,5-dimethoxy-4-morpholin-4-ylmethyl-phenyl)-carbamic acid tert-butyl ester (0.9 g, 2.74 mmol) in HOAc (20 mL) and 12 N HCl aqueous (20 mL) was stirred at 50° C. for 1 h and then concentrated to dryness using a rotary evaporator. The residue was mixed with saturated sodium bicarbonate aqueous (40 mL), extracted with DCM (3×100 mL), and concentrated. The residue was further purified by column (SiO₂, DCM/MeOH/EtOAc=3:2:3) to provide 0.6 g of 6-amino-2,4-dimethoxy-3-morpholin-4-ylmethyl-benzamide (89%). A mixture of 6-amino-2,4-dimethoxy-3-morpholin-4-ylmethyl-benzamide (0.6 g, 2.03 mmol), 3,5-dimethyl-4-hydroxy benzaldehyde (0.61 g, 4.06 mmol), sodium hydrogensulfite (1.24 g, 7.0 mmol) and p-toluenesulfonic acid (1.14 g, 6 mmol) in N,N-dimethyl acetamide (20 mL) was stirred at 115° C. for 6 h. The reaction mixture was cooled to room temperature, diluted with water (50 mL), extracted with EtOAc, and concentrated. Purification by column chromatography afforded a solid residue, which was made into slurry in a mixed solvent of DCM-hexanes (3 mL-20 mL). The slurry was filtered and washed with hexanes to provide 2-(4-hydroxy-3,5-dimethylphenyl)-5,7-dimethoxy-6-(morpholinomethyl)quinazolin-4(3H)-one (56 mg, 6.6%) as a light yellow solid. Selected data: MS (ES) m/z: 426.0; MP 237.0-239.1° C.

### Example 76

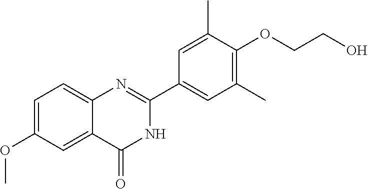 [SMILES: COc1ccc2nc(-c3cc(C)c(OCCO)c(C)c3)[nH]c(=O)c2c1]

### 2-(4-(2-hydroxyethoxy)-3,5-dimethylphenyl)-6-methoxyquinazolin-4(3H)-one

Following the method described for 6-bromo-2-(4-(2-hydroxyethoxy)-3,5-dimethylphenyl)quinazolin-4(3H)-one, 2-(4-(2-hydroxyethoxy)-3,5-dimethylphenyl)-6-methoxyquinazolin-4(3H)-one was made from 2-amino-5-methoxybenzamide and 4-(2-(tert-butyldimethylsilyloxy)ethoxy)-3-methylbenzaldehyde in 4% yield and isolated as a white solid. Selected data: ¹H NMR (300 MHz, DMSO-d₆) δ 12.28 (s, 1H), 7.88 (s, 2H), 7.68 (d, J=8.90 Hz, 1H), 7.53 (d, J=2.95 Hz, 1H), 7.43 (dd, J=8.90, 2.98 Hz, 1H), 4.89 (t, J=5.52 Hz, 1H), 3.92-3.80 (m, 5H), 3.73 (q, J=5.09, 5.09, 4.97 Hz, 2H), 2.32 (s, 6H); MS (APCI) m/z 341 [M+H]⁺.

### Example 77

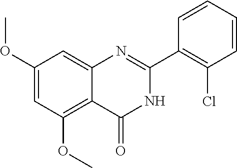 [SMILES: COc1cc(OC)c2c(=O)[nH]c(-c3ccccc3Cl)nc2c1]

### 2-(2-chlorophenyl)-5,7-dimethoxyquinazolin-4(3H)-one

A mixture of 2-chlorobenzaldehyde (0.0430 g, 306 mmol), 2-amino-4,6-dimethoxybenzamide (0.0600 g, 0.306 mmol), NaHSO₃ (94%, 0.0474 g, 0.428 mmol), and p-TsOH.H₂O (0.0175 g, 0.0918 mmol) in DMA (3.06 mL) was heated at 140° C. for 16 h. The mixture was cooled and chromatographed on silica gel, fractions containing the product were combined, concentrated under vacuum, diluted with EtOAc (300 mL), washed with water (3×75 mL), brine (75 mL), dried over sodium sulfate, filtered and concentrated under vacuum to provide 2-(2-chlorophenyl)-5,7-dimethoxyquinazolin-4(3H)-one (0.0377 g, 39%) as a yellow solid. Selected data: ¹H NMR (300 MHz, DMSO-d₆) δ 12.14 (s, 1H), 7.65-7.40 (m, 4H), 6.72 (d, J=2.29 Hz, 1H), 6.59 (d, J=2.30 Hz, 1H), 3.87 (s, 3H), 3.85 (s, 3H); MS (APCI) m/z 317 [M+H]⁺.

### Example 78

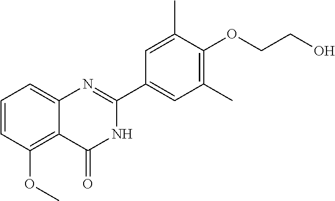 [SMILES: COc1cccc2nc(-c3cc(C)c(OCCO)c(C)c3)[nH]c(=O)c12]

### 2-(4-(2-hydroxyethoxy)-3,5-dimethylphenyl)-5-methoxyquinazolin-4(3H)-one

Following the method described for 6-bromo-2-(4-(2-hydroxyethoxy)-3,5-dimethylphenyl)quinazolin-4(3H)-one, 2-(4-(2-Hydroxyethoxy)-3,5-dimethylphenyl)-5-methoxyquinazolin-4(3H)-one was made from 2-amino-6-methoxybenzamide (made from the corresponding amino acid in two steps) and 4-(2-(tert-butyldimethylsilyloxy)ethoxy)-3-methylbenzaldehyde in 77% yield and isolated as a white solid. Selected data: ¹H NMR (300 MHz, DMSO-d₆) δ ppm 11.96 (s, 1H), 7.89 (s, 2H), 7.68 (t, J=8.20 Hz, 1H), 7.23 (d, J=7.89 Hz, 1H), 6.98 (d, J=8.19 Hz, 1H), 4.89 (t, J=5.53 Hz, 1H), 3.94-3.65 (m, 7H), 2.31 (s, 6H); MS (APCI) m/z 341 [M+H]⁺.

### Example 79

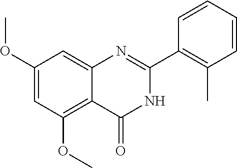 [SMILES: COc1cc(OC)c2c(=O)[nH]c(-c3ccccc3C)nc2c1]

### 5,7-dimethoxy-2-o-tolylquinazolin-4(3H)-one

A mixture of 2-amino-4,6-dimethoxybenzamide (0.060 g, 0.306 mmol), 2-methylbenzaldehyde (0.037 g, 0.306 mmol), NaHSO₃ (0.032 g, 0.306 mmol), and p-TsOH.H₂O (0.00370 g, 0.021 mmol) in DMA (5.00 mL) was heated at 60° C. overnight. The mixture was cooled to room temperature, water (50.0 mL) and EtOAc (50.0 mL) was added. The layers were separated and the organic layer was washed with water (2×50 mL), brine (50 mL), dried and concentrated. The crude solid was purified via CombiFlash provide 5,7-dimethoxy-2-o-tolylquinazolin-4(3H)-one (0.025 g, 28%) as yellow solid. Selected data: ¹H NMR (300 MHz, CDCl₃) δ 9.51 (s, 1H), 7.53 (dd, J=5.92, 3.07 Hz, 1H), 7.46-7.36 (m, 1H), 7.32 (dd, J=9.04, 4.60 Hz, 2H), 6.81 (d, J=2.29 Hz, 1H), 6.49 (d, J=2.28 Hz, 1H), 3.95 (s, J=7.48 Hz, 3H), 3.94-3.88 (s, 3H), 2.51 (s, 3H); MS (APCI) m/z 297 [M+H]⁺.

### Example 80

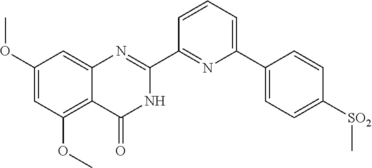 [SMILES: COc1cc(OC)c2c(=O)[nH]c(-c3cccc(-c4ccc(S(C)(=O)=O)cc4)n3)nc2c1]

### 5,7-dimethoxy-2-(6-(4-(methylsulfonyl)phenyl)pyridin-2-yl)quinazolin-4(3H)-one

Following the procedure described above for 2-(2-chlorophenyl)-5,7-dimethoxyquinazolin-4(3H)-one, 5,7-dimethoxy-2-(6-(4-(methylsulfonyl)phenyl)pyridin-2-yl)quinazolin-4(3H)-one was made from 6-(4-(methylsulfonyl)phenyl)picolinaldehyde and 2-amino-4,6-dimethoxybenzamide in 38% as a yellow solid. Selected data: ¹H NMR (300 MHz, DMSO-d₆) δ 11.75 (s, 1H), 8.69 (d, J=8.38 Hz, 2H), 8.46 (d, J=7.72 Hz, 1H), 8.33 (d, J=7.75 Hz, 1H), 8.22 (t, J=7.84 Hz, 1H), 8.08 (d, J=8.37 Hz, 2H), 6.85 (s, 1H), 6.63 (s, 1H), 3.95 (s, 3H), 3.90 (s, 3H), 3.4 (s, 3H); MS (APCI) m/z 438 [M+H]⁺.

### Example 81

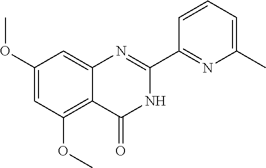 [SMILES: COc1cc(OC)c2c(=O)[nH]c(-c3cccc(C)n3)nc2c1]

### 5,7-dimethoxy-2-(6-methylpyridin-2-yl)quinazolin-4(3H)-one

Following the method described for 2-(2-chlorophenyl)-5,7-dimethoxyquinazolin-4(3H)-one, 5,7-dimethoxy-2-(6-methylpyridin-2-yl)quinazolin-4(3H)-one was made from 6-methylpicolinaldehyde and 2-amino-4,6-dimethoxybenzamide in 33% yield and isolated as an off-white solid. Selected data: ¹H NMR (300 MHz, DMSO-d₆) δ 11.00 (s, 1H), 8.21 (d, J=7.74 Hz, 1H), 7.95 (t, J=7.75 Hz, 1H), 7.52 (d, J=7.62 Hz, 1H), 6.82 (d, J=2.33 Hz, 1H), 6.60 (d, J=2.31 Hz, 1H), 3.92 (s, 3H), 3.87 (s, 3H), 2.62 (s, 3H); MS (APCI) m/z 298 [M+H]⁺.

### Example 82

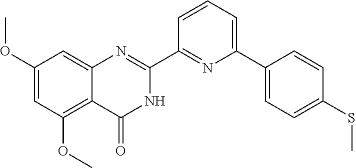 [SMILES: COc1cc(OC)c2c(=O)[nH]c(-c3cccc(-c4ccc(SC)cc4)n3)nc2c1]

### 5,7-dimethoxy-2-(6-(4-(methylthio)phenyl)pyridin-2-yl)quinazolin-4(3H)-one

Following the procedure described above for 2-(2-chlorophenyl)-5,7-dimethoxyquinazolin-4(3H)-one, 5,7-dimethoxy-2-(6-(4-(methylthio)phenyl)pyridin-2-yl)quinazolin-4(3H)-one was made from 6-(4-(methylthio)phenyl)picolinaldehyde and 2-amino-4,6-dimethoxybenzamide in 39% as a white solid. Selected data: ¹H NMR (300 MHz, DMSO-d₆) δ 11.51 (s, 1H), 8.39-8.30 (m, 3H), 8.23-8.05 (m, 2H), 7.46-7.37 (m, 2H), 6.84 (d, J=2.33 Hz, 1H), 6.62 (d, J=2.33 Hz, 1H), 3.92 (s, 3H), 3.88 (s, 3H), 2.55 (s, 3H); MS (APCI) m/z 406 [M+H]⁺.

### Example 83

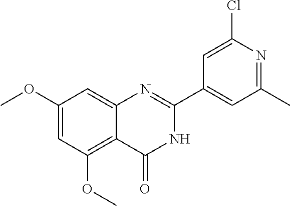

### 2-(2-chloro-6-methylpyridin-4-yl)-5,7-dimethoxyquinazolin-4(3H)-one

Following the method described for 5,7-dimethoxy-2-(4-methoxy-3,5-dimethylphenyl)quinazolin-4(3H)-one, 2-(2-chloro-6-methylpyridin-4-yl)-5,7-dimethoxyquinazolin-4(3H)-one was synthesized from 2-amino-4,6-dimethoxybenzamide and 2-chloro-6-methylisonicotinoyl chloride in 75% yield as a white solid. Selected data: ¹H NMR (300 MHz, CDCl₃) δ 10.95 (s, 1H), 7.90 (s, 2H), 6.74 (d, J=2.33 Hz, 1H), 6.51 (d, J=2.32 Hz, 1H), 3.88 (s, 3H), 3.86 (s, 3H), 2.29 (s, 3H); MS (APCI) m/z 332 [M+H]⁺.

### Example 84

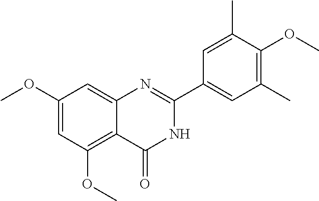

### 5,7-dimethoxy-2-(4-methoxy-3,5-dimethylphenyl)quinazolin-4(3H)-one

To a solution of 4-methoxy-3,5-dimethylbenzoic acid (0.100 g, 0.555 mmol) in CH₂Cl₂ (2.77 mL) cooled to 0-5° C. was added oxalyl chloride (67.8 μL, 0.777 mmol) followed by drop-wise addition of DMF (4.3 μL, 0.056 mmol). The mixture was stirred for 50 min, the volatiles were removed under vacuum, and the crude acid chloride was used immediately without further purification.

To a mixture of 2-amino-4,6-dimethoxybenzamide (0.0990 g, 0.555 mmol) and pyridine (44.9 μL, 0.555 mmol) in THF (2.02 mL) was added dropwise a solution of the acid chloride (crude residue described above) in THF (925 μL). After 16 h, the mixture was diluted with EtOAc (300 mL), washed with saturated aqueous NH₄Cl (3×75 mL), saturated aqueous NaHCO₃ (3×75 mL), and brine (75 mL). The insoluble yellow solid was isolated by filtration to provide the amide (0.150 g, 83%). A mixture of the amide (0.148 g, 0.413 mmol) and 2 M NaOH (7.00 mL) was heated at 85° C. for 19 h, cooled to 5° C., and neutralized with 4 M HCl in dioxanes. The white solid was filtered and rinsed with acetone to provide 5,7-dimethoxy-2-(4-methoxy-3,5-dimethylphenyl)quinazolin-4(3H)-one (0.144 g, 100%). Selected data: ¹H NMR (300 MHz, CDCl₃) δ 11.00 (s, 1H), 7.90 (s, 2H), 6.74 (d, J=2.33 Hz, 1H), 6.51 (d, J=2.32 Hz, 1H), 3.88 (s, 3H), 3.86 (s, 3H), 3.72 (s, 3H), 2.29 (s, 6H); MS (APCI) m/z 341 [M+H]⁺.

### Example 85

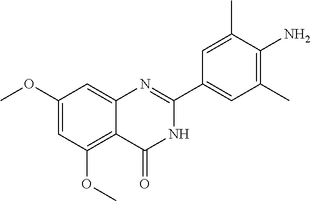

### 2-(4-amino-3,5-dimethylphenyl)-5,7-dimethoxyquinazolin-4(3H)-one

To a solution of 3,5-dimethyl-4-nitrobenzoic acid (1.00 g, 5.12 mmol) in CH₂Cl₂ (25.6 mL) cooled to 0-5° C. was added oxalyl chloride (0.626 mL, 7.17 mmol) followed by dropwise addition of DMF (39.8 μL). The mixture was stirred for 2 h, the volatiles were removed under vacuum, and the crude acid chloride was used immediately without further purification. To a mixture of 2-amino-4,6-dimethoxybenzamide (0.913 g, 4.65 mmol) and pyridine (414 μL, 5.12 mmol) in THF (18.6 mL) was added dropwise a solution of the acid chloride (crude residue described above) in THF (8.53 mL). After 16 h, the mixture was diluted with EtOAc (500 mL), washed with saturated aqueous NH₄Cl (3×100 mL), saturated aqueous NaHCO₃ (3×100 mL), and brine (100 mL). The insoluble yellow solid was isolated by filtration to provide the amide (1.51 g, 87%). A mixture of the amide (1.50 g, 4.03 mmol) and 2 M aqueous NaOH (25.0 mL) was heated at 85° C. for 17 h, then added THF (50 mL) and stirred at reflux for 25 h. The volatiles were removed under vacuum, the mixture was cooled to 5° C., and neutralized with 4 M HCl in dioxanes. After stirring for 30 min, the white solid was filtered and lyophilized from MeCN/H₂O to afford the cyclized compound (1.36 g, 95%). A mixture of the cyclized compound (0.200 g, 0.563 mmol), Na₂S₂O₄ (0.980 g, 5.63 mmol), water (5.00 mL) and MeOH (15.0 mL) was stirred at 70° C. for 2 h. The volatiles were removed under vacuum, then diluted with EtOAc (200 mL), washed with saturated NaHCO₃ (2×100 mL) and brine (75 mL). The organic layer was dried over sodium sulfate, filtered, and the volatiles were removed under vacuum to provide 2-(4-amino-3,5-dimethylphenyl)-5,7-dimethoxyquinazolin-4(3H)-one (0.062 g, 34%) as a yellow solid. Selected data: ¹H NMR (300 MHz, DMSO-d₆) δ 11.45 (s, 1H), 7.78 (s, 2H), 6.66 (d, J=2.25 Hz, 1H), 6.42 (d, J=2.24 Hz, 1H), 5.26 (s, 2H), 3.88 (s, 3H), 3.86 (s, 3H), 2.14 (s, 6H); MS (APCI) m/z 326 [M+H]⁺.

### Example 86

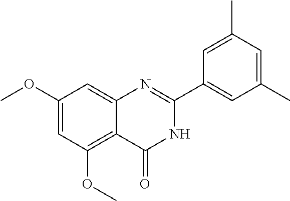

### 2-(3,5-dimethylphenyl)-5,7-dimethoxyquinazolin-4(3H)-one

A mixture of 2-amino-4,6-dimethoxybenzamide (0.0700 g, 0.36 mmol) and 3,5-dimethylbenzoyl chloride (0.112 g, 0.65 mmol) in THF (5.0 mL) was placed in a microwave reactor at 80° C. for 30 min. The THF was removed under reduced pressure, and the residue was purified via CombiFlash chromatography to yield the expected amide. This material was used directly in the next step. A mixture of the amide and H₂O/MeCN (2:1, 5.00 mL) was basified to pH 12 with 2 N NaOH and stirred at 80° C. for 16 h. The mixture was cooled and neutralized with 1 N HCl. The resulting precipitate was collected on a frit, washed with water (5.00 mL) and lyophilized to yield 2-(3,5-dimethylphenyl)-5,7-dimethoxyquinazolin-4(3H)-one (0.0395 g, 31% over two steps) as a white solid. Selected data: ¹H NMR (300 MHz, DMSO-d₆) δ 11.88 (s, 1H), 7.80 (s, 2H), 7.21 (s, 1H), 6.76 (d, J=2.24 Hz, 1H), 6.53 (d, J=2.21 Hz, 1H), 3.89 (s, 3H), 3.85 (s, 3H), 2.35 (s, 6H); MS (APCI) m/z 311 [M+H]⁺.

### Example 87

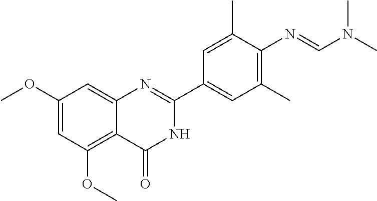

### (E)-N′-(4-(5,7-dimethoxy-4-oxo-3,4-dihydroquinazolin-2-yl)-2,6-dimethylphenyl)-N,N-dimethylformimidamide

To a solution of 2-(4-amino-3,5-dimethylphenyl)-5,7-dimethoxyquinazolin-4(3H)-one (0.096 g, 0.295 mmol) and diisopropylethylamine (61.7 μL, 0.354 mmol), in DMF (2.96 mL) was added dropwise methanesulfonyl chloride (25.2 μL, 0.325 mmol). After stirring at room temperature for 18 h, the mixture was diluted with EtOAc (300 mL), washed with saturated aqueous sodium bicarbonate (2×75 mL), saturated aqueous LiCl (2×75 mL), dried over sodium sulfate, filtered and concentrated under vacuum. The residue was purified over silica gel (12 g, CH₂Cl₂/CH₃OH) to provide (E)-N′-(4-(5,7-dimethoxy-4-oxo-3,4-dihydroquinazolin-2-yl)-2,6-dimethylphenyl)-N,N-dimethylformimidamide (0.0502 g, 45%) as a white solid. Selected data: ¹H NMR (300 MHz, DMSO-d₆) δ 11.68 (s, 1H), 7.87 (s, 2H), 7.40 (s, 1H), 6.72 (d, J=2.31 Hz, 1H), 6.48 (d, J=2.31 Hz, 1H), 3.88 (s, 3H), 3.86 (s, 3H), 2.97 (s, 6H), 2.12 (s, 6H); MS (APCI) m/z 381 [M+H]⁺.

### Example 88

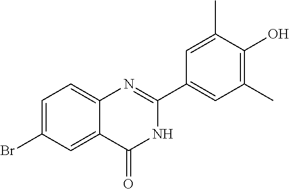 [SMILES: Cc1cc(-c2nc3ccc(Br)cc3c(=O)[nH]2)cc(C)c1O]

### 6-bromo-2-(4-hydroxy-3,5-dimethylphenyl)quinazolin-4(3H)-one

To a solution of 4-hydroxy-3,5-dimethylbenzoic acid (2.00 g, 12.0 mmol) in CH₂Cl₂ (60.2 mL) cooled to 0-5° C. was added oxalyl chloride (1.47 mL, 16.8 mmol) followed by dropwise addition of DMF (93.3 μL, 1.20 mmol). The mixture was stirred for 1.25 h, the volatiles were removed under vacuum to give crude acid chloride, which was used immediately without further purification. A mixture of 2-amino-5-bromobenzamide (1.99 g, 9.23 mmol) and the acid chloride (crude residue described above) in THF (92.3 mL) was stirred at room temperature for 17 h, then heated at reflux for 4 h. The volatiles were removed under vacuum, the residue was triturated with EtOAc, and filtered to afford the amide (3.02 g, 90%) as a yellow solid. A mixture of the amide (3.01 g, 8.29 mmol), 2 M NaOH (20.0 mL), water (40.0 mL), and MeCN (20.0 mL) was heated at reflux for 15 h, cooled to 5° C., and neutralized with 2 M aqueous HCl. After stirring for 30 min, the white solid was filtered, triturated with acetone, and filtered again to afford 6-bromo-2-(4-hydroxy-3,5-dimethylphenyl)quinazolin-4(3H)-one (2.28 g, 80%). Selected data: ¹H NMR (300 MHz, DMSO-d₆) δ 8.18 (d, J=2.29 Hz, 1H), 7.93 (dd, J=8.72, 2.42 Hz, 1H), 7.86 (s, 2H), 7.63 (d, J=8.70 Hz, 1H), 5.75 (s, 1H), 2.24 (s, 6H); MS (APCI) m/z 346 [M+H]⁺.

### Example 89

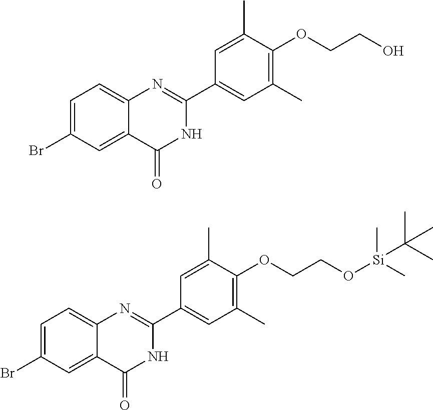 [SMILES: Cc1cc(-c2nc3ccc(Br)cc3c(=O)[nH]2)cc(C)c1OCCO ; Cc1cc(-c2nc3ccc(Br)cc3c(=O)[nH]2)cc(C)c1OCCO[Si](C)(C)C(C)(C)C]

### 6-bromo-2-(4-(2-hydroxyethoxy)-3,5-dimethylphenyl)quinazolin-4(3H)-one (left) and 6-bromo-2-(4-(2-(tert-butyldimethylsilyloxy)ethoxy)-3,5-dimethylphenyl)quinazolin-4(3H)-one (right)

A mixture of 2-amino-5-bromobenzamide (0.100 g, 0.465 mmol), 4-(2-(tert-butyldimethylsilyloxy)ethoxy)-3-methylbenzaldehyde (0.143 g, 0.465 mmol), NaHSO₃ (94%, 0.0515 g, 0.465 mmol), and p-TsOH.H₂O (0.00885 g, 0.0465 mmol) in DMA (5.81 mL) was heated at reflux for 15 min, cooled to room temperature, the water (20 mL) was added. The precipitate was filtered, washed with water, triturated with acetone and filtered again. The crude solid was chromatographed on silica gel (CH₂Cl₂/CH₃OH) to provide 6-bromo-2-(4-(2-hydroxyethoxy)-3,5-dimethylphenyl)quinazolin-4(3H)-one (0.0395 g, 22%) and 6-bromo-2-(4-(2-(tert-butyldimethylsilyloxy)ethoxy)-3,5-dimethylphenyl)quinazolin-4(3H)-one (0.0227 g, 10%) as white solids. Selected data for 6-bromo-2-(4-(2-hydroxyethoxy)-3,5-dimethylphenyl)quinazolin-4(3H)-one: ¹H NMR (300 MHz, DMSO-d₆) δ 12.48 (s, 1H), 8.20 (d, J=2.34 Hz, 1H), 8.01-7.80 (m, 3H), 7.66 (d, J=8.72 Hz, 1H), 4.90 (t, J=5.46 Hz, 1H), 3.85 (t, J=4.87 Hz, 2H), 3.73 (dd, J=10.06, 5.11 Hz, 2H), 2.32 (s, 6H); MS (APCI) m/z 345 [M+H]⁺. Selected data for 6-bromo-2-(4-(2-(tert-butyldimethylsilyloxy)ethoxy)-3,5-dimethylphenyl)quinazolin-4(3H)-one: ¹H NMR (300 MHz, DMSO-d₆) δ 12.49 (s, 1H), 8.20 (d, J=2.34 Hz, 1H), 7.95 (dd, J=8.71, 2.41 Hz, 1H), 7.90 (s, 2H), 7.67 (d, J=8.72 Hz, 1H), 3.90 (m, 4H), 2.32 (s, 6H), 0.90 (s, 9H), 0.09 (s, 6H); MS (APCI) m/z 503 [M+H]⁺.

### Example 90

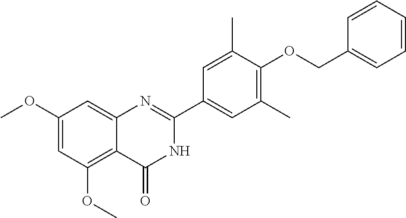 [SMILES: COc1cc(OC)c2c(=O)[nH]c(-c3cc(C)c(OCc4ccccc4)c(C)c3)nc2c1]

### 2-(4-(benzyloxy)-3,5-dimethylphenyl)-5,7-dimethoxyquinazolin-4(3H)-one

Following the method described for 2-(3-tert-butyl-1-methyl-1H-pyrazol-5-yl)-5,7-dimethoxyquinazolin-4(3H)-one, compound 2-(4-(benzyloxy)-3,5-dimethylphenyl)-5,7-dimethoxyquinazolin-4(3H)-one was synthesized from 2-amino-4,6-dimethoxybenzamide and 4-(benzyloxy)-3,5-dimethylbenzoyl chloride in 7% yield as a white solid. Selected data: ¹H NMR (300 MHz, DMSO-d₆) δ 11.84 (s, 1H), 7.93 (s, 2H), 7.57-7.33 (m, 5H), 6.75 (d, J=2.28 Hz, 1H), 6.52 (d, J=2.27 Hz, 1H), 4.88 (s, 2H), 3.88 (s, 3H), 3.86 (s, 3H), 2.31 (s, 6H); MS (APCI) m/z 417 [M+H]⁺.

### Example 91

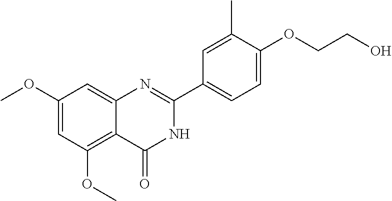 [SMILES: COc1cc(OC)c2c(=O)[nH]c(-c3ccc(OCCO)c(C)c3)nc2c1]

### 2-(4-(2-hydroxyethoxy)-3-methylphenyl)-5,7-dimethoxyquinazolin-4(3H)-one

A mixture of 3-methyl-4-hydroxybenzaldehyde (0.200 g, 1.5 mmol), (2-bromoethoxy)-tert-butyldimethylsilane (0.538 g, 2.25 mmol) and sodium hydride (0.061 g, 2.55 mmol) in DMF (5.00 mL) was stirred open at room temperature for 30 min in a microwave vial. The vial was then capped and heated in the microwave reactor for 1 h at 80° C. Water (55.0 mL) was added to quench. The solution was diluted with 1 N HCl (25.0 mL) and extracted with EtOAc (2×25.0 mL), dried and evaporated. The crude material was purified via CombiFlash to yield the alkylated aldehyde. A mixture of 2-amino-4,6-dimethoxybenzamide (0.167 g, 0.85 mmol), 4-(2-(tert-butyldimethylsilyloxy)ethoxy)-3-methylbenzaldehyde (0.250 g, 0.85 mmol), p-TsOH.H₂O (0.016 g, 0.085 mmol) and NaHSO₃ (0.088 g, 0.85 mmol) in DMA (5.00 mL) was stirred at 155° C. for 90 min. The solution was diluted with EtOAc (150 mL), washed with saturated NaHCO₃ (2×50 mL), 1 N HCl (2×75 mL), brine (50 mL), dried and the solvent was removed under reduced pressure to yield the TBS protected material (0.068 g, 17%) as a tan solid. The crude material was used directly in the next step. The TBS-protected material (0.068 g, 0.144 mmol) and 1 M TBAF in THF (1.00 mL, 7 mmol) was stirred at room temperature for 1 h. The volatiles were removed under vacuum, and the residue diluted with EtOAc (100 mL). The solution was washed with water (2×50.0 mL), brine (50.0 mL), dried and the solvent was removed. The residue was purified via CombiFlash to yield 2-(4-(2-hydroxyethoxy)-3-methylphenyl)-5,7-dimethoxyquinazolin-4(3H)-one (0.024 g, 47%) as an orange solid. Selected data: ¹H NMR (300 MHz, DMSO-d₆) δ 11.81 (s, 1H), 8.05 (m, 2H), 7.05 (d, 1H, J=8.3 Hz), 6.72 (d, 1H, J=2.2 Hz), 6.50 (d, 1H, J=2.2 Hz), 4.87 (t, 1H, J=5.5 Hz), 4.09 (t, 2H, J=4.9 Hz), 3.89 (s, 3H), 3.84 (s, 3H), 3.76 (dd, 2H, J=5.1 Hz, J=10.0 Hz), 2.24 (s, 3H); MS (APCI) m/z 357 [M+H]⁺.

### Example 92

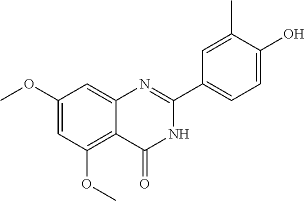 [SMILES: COc1cc(OC)c2c(=O)[nH]c(-c3ccc(O)c(C)c3)nc2c1]

### 2-(4-hydroxy-3-methylphenyl)-5,7-dimethoxyquinazolin-4(3H)-one

A mixture of 4-hydroxy-3-methylbenzaldehyde (0.200 g, 1.47 mmol), 2-amino-4,6-dimethoxybenzamide (0.288 g, 1.47 mmol), NaHSO₃ (94%, 0.163 g, 1.47 mmol), and p-TsOH.H₂O (0.028 g, 0.147 mmol) in DMA (18.4 mL) was heated at reflux for 1 h. The mixture was diluted with EtOAc (300 mL), washed with saturated aqueous NH₄Cl (2×150 mL) and brine (75 mL), dried over sodium sulfate, filtered and concentrated under vacuum. The residue was triturated with MeOH and filtered off a yellow solid, which was freeze-dried from MeCN/H₂O to provide 2-(4-hydroxy-3-methylphenyl)-5,7-dimethoxyquinazolin-4(3H)-one (0.161 g, 35%). Selected data: ¹H NMR (300 MHz, DMSO-d₆) δ 11.71 (s, 1H), 10.02 (s, 1H), 7.99 (d, J=1.88 Hz, 1H), 7.89 (dd, J=8.47, 2.29 Hz, 1H), 6.86 (d, J=8.50 Hz, 1H), 6.69 (d, J=2.31 Hz, 1H), 6.48 (d, J=2.31 Hz, 1H), 3.88 (s, 3H), 3.86 (s, 3H), 2.18 (s, 3H); MS (APCI) m/z 313 [M+H]⁺.

### Example 93

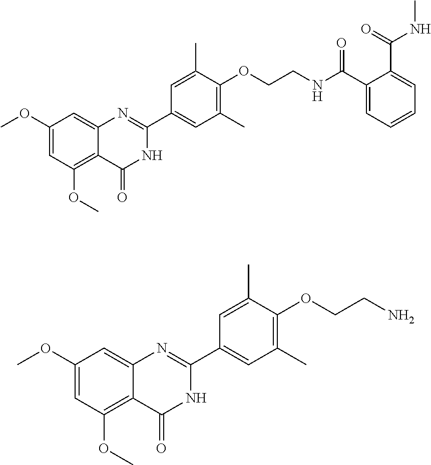

### N1-(2-(4-(5,7-dimethoxy-4-oxo-3,4-dihydroquinazolin-2-yl)-2,6-dimethylphenoxy)ethyl)-N2-methylphthalamide (left) and 2-(4-(2-aminoethoxy)-3,5-dimethylphenyl)-5,7-dimethoxyquinazolin-4(3H)-one (right)

A mixture of 3,5-dimethyl-4-hydroxybenzaldehyde (0.600 g, 4.00 mmol), N-(2-bromoethyl)-phthalimide (1.22 g, 4.80 mmol), K₂CO₃ (0.829 g, 6.00 mmol), NaI (3.00 g, 20.0 mmol) in DMF (40.0 mL) was heated at 80° C. for 2.5 h. The reaction was cooled to room temperature, diluted with EtOAc (200 mL), washed with 1 M NaOH (2×100 mL), 1 M HCl (2×100 mL), brine (75 mL), dried over sodium sulfate, filtered and concentrated under vacuum. The residue was chromatographed on silica gel (40 g, hexanes/EtOAc) to provide the expected ether (0.300 g, 23%) as a yellow solid. A mixture of the above ether (0.293 g, 0.907 mmol), 2-amino-4,6-dimethoxybenzamide (0.178 g, 0.907 mmol), NaHSO₃ (94%, 0.100 g, 0.907 mmol), and p-TsOH.H₂O (0.0173 g, 0.0907 mmol) in DMA (11.3 mL) was stirred at reflux for 1.5 h then cooled to room temperature. The mixture was diluted with EtOAc (250 mL), washed with saturated aqueous ammonium chloride (3×75 mL) and brine (75 mL), dried over sodium sulfate, filtered and concentrated under vacuum. The residue was chromatographed on silica gel (40 g, CH₂Cl₂/CH₃OH) to provide the expected product (0.075 g, 17%) as a light yellow solid. A mixture of the above compound (0.213 g, 0.426 mmol) and 2 M methylamine in THF (25.0 mL) was stirred at room temperature for 17 h. The volatiles were removed under vacuum and the residue was chromatographed on silica gel to provide compound N1-(2-(4-(5,7-dimethoxy-4-oxo-3,4-dihydroquinazolin-2-yl)-2,6-dimethylphenoxy)ethyl)-N2-methylphthalamide (0.0493 g, 22%) and compound 2-(4-(2-aminoethoxy)-3,5-dimethylphenyl)-5,7-dimethoxyquinazolin-4(3H)-one (0.0360 g, 23%) as white solids. Selected data for N1-(2-(4-(5,7-dimethoxy-4-oxo-3,4-dihydroquinazolin-2-yl)-2,6-dimethylphenoxy)ethyl)-N2-methylphthalamide: ¹H NMR (300 MHz, DMSO-d₆) δ 11.80 (s, 1H), 8.51 (t, J=5.57 Hz, 1H), 8.18 (q, J=4.57 Hz, 1H), 7.89 (s, 2H), 7.53-7.42 (m, 4H), 6.74 (d, J=2.31 Hz, 1H), 6.52 (d, J=2.29 Hz, 1H), 3.96-3.80 (m, 8H), 3.61 (q, J=5.73 Hz, 2H), 2.71 (d, J=4.62 Hz, 3H), 2.32 (s, 6H); MS (APCI) m/z 531 [M+H]⁺. Selected data for 2-(4-(2-aminoethoxy)-3,5-dimethylphenyl)-5,7-dimethoxyquinazolin-4(3H)-one: ¹H NMR (300 MHz, DMSO-d₆) δ 7.90 (s, 2H), 6.74 (d, J=2.31 Hz, 1H), 6.51 (d, J=2.32 Hz, 1H), 3.88 (s, 3H), 3.85 (s, 3H), 3.77 (t, J=5.76 Hz, 2H), 2.91 (t, J=5.75 Hz, 2H), 2.30 (s, 6H); MS (APCI) m/z 370 [M+H]⁺.

### Example 94

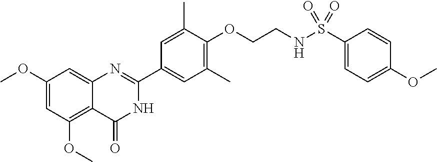

### N-(2-(4-(5,7-dimethoxy-4-oxo-3,4-dihydroquinazolin-2-yl)-2,6-dimethylphenoxy)ethyl)-4-methoxybenzenesulfonamide

A mixture of 2-(4-(2-aminoethoxy)-3,5-dimethylphenyl)-5,7-dimethoxyquinazolin-4(3H)-one (0.060 g, 0.162 mmol), 4-methoxybenzenesulfonyl chloride (0.044 mg, 0.211 mmol), and triethylamine (29.4 μL, 0.211 mmol) in CH₂Cl₂ (812 μL) was stirred at room temperature for 3 h. The mixture was chromatographed directly on silica gel to yield N-(2-(4-(5,7-dimethoxy-4-oxo-3,4-dihydroquinazolin-2-yl)-2,6-dimethylphenoxy)ethyl)-4-methoxybenzenesulfonamide (0.046 g, 53%) as a white solid after lyophilization from MeCN/H₂O. Selected data: ¹H NMR (300 MHz, DMSO-d₆) δ ppm 11.81 (s, 1H), 7.88 (s, 2H), 7.83-7.73 (m, 3H), 7.17-7.07 (m, 2H), 6.73 (d, J=2.31 Hz, 1H), 6.52 (d, J=2.29 Hz, 1H), 3.91-3.75 (m, 11H), 3.12 (q, J=5.75 Hz, 2H), 2.24 (s, 6H); MS (APCI) m/z 540 [M+H]⁺.

### Example 95

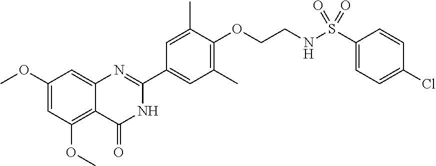 [SMILES: COc1cc(OC)c2c(=O)[nH]c(-c3cc(C)c(OCCNS(=O)(=O)c4ccc(Cl)cc4)c(C)c3)nc2c1]

### 4-chloro-N-(2-(4-(5,7-dimethoxy-4-oxo-3,4-dihydroquinazolin-2-yl)-2,6-dimethylphenoxy)ethyl)benzenesulfonamide

Following the method described for N-(2-(4-(5,7-dimethoxy-4-oxo-3,4-dihydroquinazolin-2-yl)-2,6-dimethylphenoxy)ethyl)-4-methoxybenzenesulfonamide, compound 4-chloro-N-(2-(4-(5,7-dimethoxy-4-oxo-3,4-dihydroquinazolin-2-yl)-2,6-dimethylphenoxy)ethyl)benzene-sulfonamide was made from 2-(4-(2-aminoethoxy)-3,5-dimethylphenyl)-5,7-dimethoxyquinazolin-4(3H)-one in 51% yield and isolated as a white solid after lyophilization from MeCN/H₂O. Selected data: ¹H NMR (300 MHz, DMSO-d₆) δ ppm 11.8 (s, 1H), 8.1 (s, 1H), 7.9-7.6 (m, 6H), 6.75 (1H), 6.5 (1H), 3.9-3.7 (m, 8H), 3.15 (m, 2H), 2.2 (s, 6H); MS (APCI) m/z 544 [M+H]⁺.

### Example 96

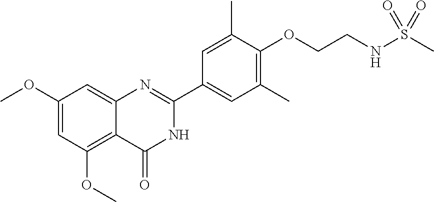

### N-(2-(4-(5,7-dimethoxy-4-oxo-3,4-dihydroquinazolin-2-yl)-2,6-dimethylphenoxy)ethyl)methanesulfonamide

Following the method described for N-(2-(4-(5,7-dimethoxy-4-oxo-3,4-dihydroquinazolin-2-yl)-2,6-dimethylphenoxy)ethyl)-4-methoxybenzenesulfonamide, compound N-(2-(4-(5,7-dimethoxy-4-oxo-3,4-dihydroquinazolin-2-yl)-2,6-dimethylphenoxy)ethyl)methanesulfonamide was made from 2-(4-(2-aminoethoxy)-3,5-dimethylphenyl)-5,7-dimethoxyquinazolin-4(3H)-one in 42% yield and isolated as a white solid after lyophilization from MeCN/H₂O. Selected data: ¹H NMR (300 MHz, DMSO-d₆) δ ppm 11.82 (s, 1H), 7.90 (s, 2H), 7.33 (t, J=5.94 Hz, 1H), 6.74 (d, J=2.31 Hz, 1H), 6.52 (d, J=2.30 Hz, 1H), 3.92-3.81 (m, 8H), 3.41-3.34 (m, 2H), 2.97 (s, 3H), 2.32 (s, 6H); MS (APCI) m/z 448 [M+H]⁺.

### Example 97

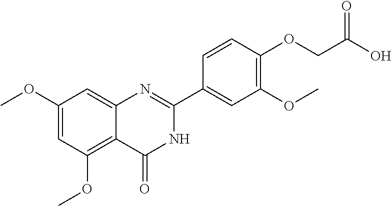 [SMILES: COc1cc(OC)c2c(=O)[nH]c(-c3ccc(OCC(=O)O)c(OC)c3)nc2c1]

### 2-(4-(5,7-dimethoxy-4-oxo-3,4-dihydroquinazolin-2-yl)-2-methoxyphenoxy)acetic acid

A mixture of NaOH (1.8 g, 0.045 mol) and 4-hydroxy-3-methoxylbenzalde (3.10 g, 0.0203 mol) in water (20 mL) was mixed with bromoacetic acid (2.82 g, 0.0203 mol) and heated to reflux for 6 h. The reaction mixture was adjusted to pH 3.0 by adding a HCl solution. The solid was filtered off and further washed with cold water and ethyl acetate (2×30 mL) to yield (4-formyl-2-methyl-phenoxy)-acetic acid (2.89 g, 67.7%). 2-Amino-4,6-dimethoxy-benzamide (150 mg, 0.764 mmol) with (4-formyl-2-methyl-phenoxy)-acetic acid (160 mg, 0.764 mmol), sodium hydrogen sulfite (150 mg, 58.5%) and p-toluenesulfonic acid monohydrate (15 mg) in N,N-dimethyl acetamide (10 mL) were heated to 150° C. for 16 h. N,N-dimethyl acetamide was removed under vacuum and the residue was poured into water (50 mL). The solid was filtered off and further purified by base/acid extractions/washes to yield 2-(4-(5,7-dimethoxy-4-oxo-3,4-dihydroquinazolin-2-yl)-2-methoxyphenoxy)acetic acid (25 mg, 8.1%). Selected data: MS (ES) m/z: 387.1; MP 275-277° C.

### Example 98

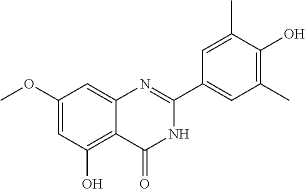 [SMILES: COc1cc(O)c2c(=O)[nH]c(-c3cc(C)c(O)c(C)c3)nc2c1]

### 5-hydroxy-2-(4-hydroxy-3,5-dimethylphenyl)-7-methoxyquinazolin-4(3H)-one

A mixture of 2-amino-4,6-dimethoxy-benzamide (0.71 g, 3.71 mmol), 3,5-dimethyl-4-benzyloxy benzaldehyde (0.94 g, 3.90 mmol), sodium hydrogensulfite (0.68 g, 3.90 mmol) and p-toluenesulfonic acid (70 mg, 0.37 mmol) in N,N-dimethylacetamide (25 mL) was stirred at 150° C. for 16 h. The reaction mixture was cooled to room temperature and diluted with water (200 mL). The resulting solid was collected by filtration and washed with hexanes to afford 2-(4-benzyloxy-3,5-dimethyl-phenyl)-5,7-dimethoxy-3H-quinazolin-4-one as a white solid (1.2 g, 79%).

A mixture of 2-(4-benzyloxy-3,5-dimethyl-phenyl)-5,7-dimethoxy-3H-quinazolin-4-one (1.2 g, 2.92 mmol) and magnesium bromide (0.644 g, 3.5 mmol) in pyridine (50 mL) was stirred at reflux for 12 h. The mixture was concentrated and the solid residue was made into slurry with HCl (2 N, 100 mL). The solid was collected by filtration, washed with water and hexanes to yield 2-(4-benzyloxy-3,5-dimethyl-phenyl)-5-hydroxy-7-methoxy-3H-quinazolin-4-one as a white solid (0.76 g, 65%). A solution of ammonium formate (0.945 g, 15 mmol) and 2-(4-benzyloxy-3,5-dimethyl-phenyl)-5-hydroxy-7-methoxy-3H-quinazolin-4-one (0.1 g, 0.25 mmol) in DMF (50 mL) was mixed with palladium carbon (0.1 g) and stirred at 85° C. for 14 h. The resulting suspension was cooled to room temperature, passed through a Celite pad, and washed with DCM. The filtrate was concentrated and the residue was diluted with water (20 mL). The resulting solid was collected by filtration and washed with hexanes to afford 5-hydroxy-2-(4-hydroxy-3,5-dimethylphenyl)-7-methoxyquinazolin-4(3H)-one (57 mg, 74%) as light yellow solid. Selected data: MS (ES) m/z: 312.94; MP 291.3-293° C.

### Example 99

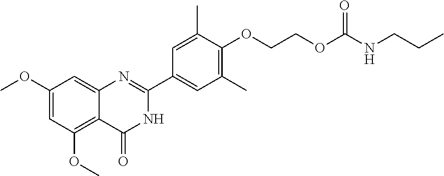 [SMILES: CCCNC(=O)OCCOc1c(C)cc(-c2nc3cc(OC)cc(OC)c3c(=O)[nH]2)cc1C]

### 2-(4-(5,7-Dimethoxy-4-oxo-3,4-dihydroquinazolin-2-yl)-2,6-dimethyl-phenoxy)ethyl propylcarbamate

A mixture of the compound of Example 20 (0.070 g, 0.19 mmol), propyl isocyanate (0.088 mL, 0.94 mmol), and TEA (0.14 g, 1.1 mmol) in THF (4.0 mL) was stirred at 70° C. for 16 h. The mixture was filtered, washed with THF, and the solvent was removed under reduced pressure. The residue was dissolved in EtOAc (50 mL) and washed with saturated aqueous sodium bicarbonate (50 mL), dried and the solvent was removed under reduced pressure. The resulting solid was chromatographed on silica gel to yield 2-(4-(5,7-Dimethoxy-4-oxo-3,4-dihydroquinazolin-2-yl)-2,6-dimethyl-phenoxy)ethyl propylcarbamate (0.035 g, 41%) as an off-white solid: Selected data: ¹H NMR (300 MHz, DMSO-d₆) δ 11.82 (s, 1H), 7.90 (s, 2H), 7.23 (t, J=5.27 Hz, 1H), 6.74 (d, J=2.32 Hz, 1H), 6.52 (d, J=2.31 Hz, 1H), 4.27 (t, J=4.29 Hz, 2H), 3.99 (t, J=4.29 Hz, 2H), 3.89 (s, 3H), 3.84 (s, 3H), 3.02-2.86 (m, 2H), 2.29 (s, 6H), 1.50-1.30 (m, 2H), 0.84 (t, J=7.33 Hz, 3H); MS (APCI) m/z 456 [M+H]⁺.

### Example 100

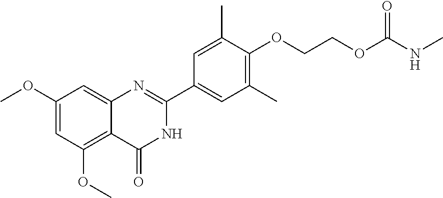

### 2-(4-(5,7-Dimethoxy-4-oxo-3,4-dihydroquinazolin-2-yl)-2,6-dimethyl-phenoxy)ethyl methylcarbamate

Following the method described for 2-(4-(5,7-Dimethoxy-4-oxo-3,4-dihydroquinazolin-2-yl)-2,6-dimethyl-phenoxy)ethyl propylcarbamate, compound 2-(4-(5,7-Dimethoxy-4-oxo-3,4-dihydroquinazolin-2-yl)-2,6-dimethyl-phenoxy)ethyl methylcarbamate was made from 2-(4-(2-hydroxyethoxy)-3,5-dimethylphenyl)-5,7-dimethoxyquinazolin-4(3H)-one in 11% yield and isolated as an off-white solid: ¹H NMR (300 MHz, DMSO-d₆) δ 11.82 (s, 1H), 7.90 (s, 2H), 7.08 (m, 1H), 6.74 (d, J=2.29 Hz, 1H), 6.52 (d, J=2.27 Hz, 1H), 4.27 (t, J=4.55 Hz, 2H), 3.99 (t, J=4.55 Hz, 2H), 3.89 (s, 3H), 3.84 (s, 3H), 2.60 (d, J=4.57 Hz, 3H), 2.29 (s, 6H); MS (APCI) m/z 428 [M+H]⁺.

### Example 101

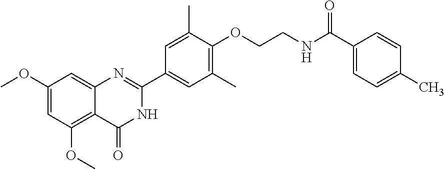

### N-(2-(4-(5,7-Dimethoxy-4-oxo-3,4-dihydroquinazolin-2-yl)-2,6-dimethylphenoxy)ethyl)-4-methylbenzamide

A mixture of compound 2-(4-(2-aminoethoxy)-3,5-dimethylphenyl)-5,7-dimethoxyquinazolin-4(3H)-one (0.060 g, 0.16 mmol), p-toluoyl chloride (0.028 mL, 0.21 mmol), and PS-DIEA (0.057 g, 0.21 mmol) in CH₂Cl₂ (4.0 mL) was stirred at room temperature for 16 h. The mixture was filtered, washed with CH₂Cl₂ and the solvent was removed under reduced pressure. The resulting residue was chromatographed on silica gel to yield N-(2-(4-(5,7-Dimethoxy-4-oxo-3,4-dihydroquinazolin-2-yl)-2,6-dimethylphenoxy)ethyl)-4-methylbenzamide (0.037 g, 51%) as an off-white solid: ¹H NMR (300 MHz, DMSO-d₆) δ 11.80-11.00 (s, 1H), 8.69 (t, J=5.43 Hz, 1H), 7.88 (s, 2H), 7.79 (d, J=8.19 Hz, 2H), 7.28 (d, J=8.00 Hz, 2H), 6.73 (d, J=2.31 Hz, 1H), 6.51 (d, J=2.31 Hz, 1H), 3.94 (t, J=5.59 Hz, 2H), 3.88 (s, 3H), 3.84 (s, 3H), 3.72-3.60 (m, 2H), 2.36 (s, 3H), 2.27 (s, 6H); MS (APCI) m/z 488 [M+H]⁺.

### Example 102

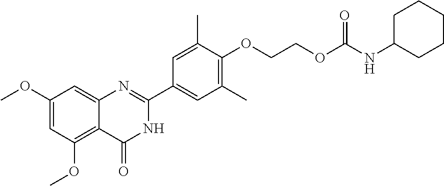 [SMILES: COc1cc(OC)c2c(=O)[nH]c(-c3cc(C)c(OCCOC(=O)NC4CCCCC4)c(C)c3)nc2c1]

### 2-(4-(5,7-dimethoxy-4-oxo-3,4-dihydroquinazolin-2-yl)-2,6-dimethylphenoxy)ethyl cyclohexylcarbamate

A mixture of Example 18 (0.100 g, 0.270 mmol), cyclohexylisocyanate (172 μL, 1.35 mmol), and Et₃N (263 μL, 1.89 mmol) in THF (1.00 mL) was stirred at reflux for 4 h then diluted with EtOAc (200 mL) and washed with saturated aqueous ammonium chloride (3×75 mL) and brine (75 mL). The organic layer was dried over sodium sulfate, filtered and concentrated under vacuum. The residue was chromatographed on silica gel (12 g, CH₂Cl₂/CH₃OH) and the product freeze dried from MeCN/H₂O to provide 2-(4-(5,7-dimethoxy-4-oxo-3,4-dihydroquinazolin-2-yl)-2,6-dimethylphenoxy)ethyl cyclohexylcarbamate (0.0981 g, 73%) as a white solid. ¹H NMR (300 MHz, DMSO-d₆ δ) 11.82 (s, 1H), 7.90 (s, 2H), 7.24-7.05 (m, 1H), 6.73 (d, J=2.30 Hz, 1H), 6.52 (d, J=2.31 Hz, 1H), 4.30-4.22 (m, 1H), 4.03-3.95 (m, 1H), 3.88 (s, 3H), 3.85 (s, 3H), 2.29 (s, 6H), 1.82-1.46 (m, 5H), 1.18 (m, 5H); MS (APCI) m/z 496 [M+H]⁺.

### Example 103

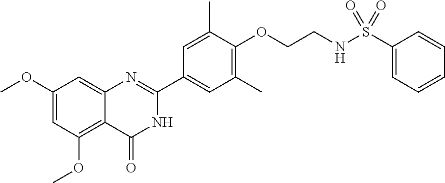 [SMILES: COc1cc(OC)c2c(=O)[nH]c(-c3cc(C)c(OCCNS(=O)(=O)c4ccccc4)c(C)c3)nc2c1]

### N-(2-(4-(5,7-dimethoxy-4-oxo-3,4-dihydroquinazolin-2-yl)-2,6-dimethylphenoxy)ethyl)benzenesulfonamide

Following the methodology described for Example 100, the title compound was made from 2-(4-(2-aminoethoxy)-3,5-dimethylphenyl)-5,7-dimethoxyquinazolin-4(3H)-one in 41% yield and isolated as an off-white solid: MS (APCI) m/z 510 [M+H]⁺.

### Example 104

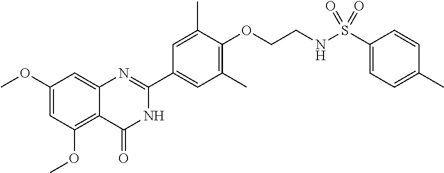

### N-(2-(4-(5,7-dimethoxy-4-oxo-3,4-dihydroquinazolin-2-yl)-2,6-dimethylphenoxy)ethyl)-4-methylbenzenesulfonamide

Following the methodology described for Example 100, the title compound was made from 2-(4-(2-aminoethoxy)-3,5-dimethylphenyl)-5,7-dimethoxyquinazolin-4(3H)-one in 50% yield and isolated as an off-white solid: MS (APCI) m/z 524 [M+H]⁺.

### Example 105

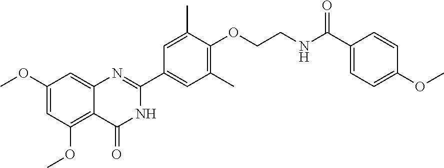

### N-(2-(4-(5,7-dimethoxy-4-oxo-3,4-dihydroquinazolin-2-yl)-2,6-dimethylphenoxy)ethyl)-4-methoxybenzamide

Following the methodology described for Example 107, the title compound was made from 2-(4-(2-aminoethoxy)-3,5-dimethylphenyl)-5,7-dimethoxyquinazolin-4(3H)-one in 46% yield and isolated as a white solid: MS (APCI) m/z 526 [M+Na]⁺.

### Example 106

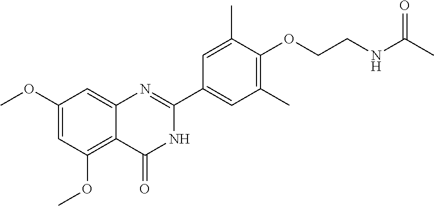 [SMILES: COc1cc(OC)c2c(=O)[nH]c(-c3cc(C)c(OCCNC(C)=O)c(C)c3)nc2c1]

### N-(2-(4-(5,7-dimethoxy-4-oxo-3,4-dihydroquinazolin-2-yl)-2,6-dimethylphenoxy)ethyl)acetamide

Following the methodology described for Example 107, the title compound was made from 2-(4-(2-aminoethoxy)-3,5-dimethylphenyl)-5,7-dimethoxyquinazolin-4(3H)-one in 40% yield and isolated as a white solid: MS (APCI) m/z 412 [M+H]⁺.

### Example 107

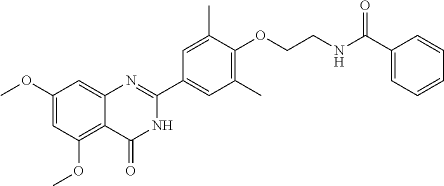 [SMILES: COc1cc(OC)c2c(=O)[nH]c(-c3cc(C)c(OCCNC(=O)c4ccccc4)c(C)c3)nc2c1]

### N-(2-(4-(5,7-dimethoxy-4-oxo-3,4-dihydroquinazolin-2-yl)-2,6-dimethylphenoxy)ethyl)benzamide

Following the methodology described for Example 107, the title compound was made from 2-(4-(2-aminoethoxy)-3,5-dimethylphenyl)-5,7-dimethoxyquinazolin-4(3H)-one in 66% yield and isolated as a white solid: MS (APCI) m/z 474 [M+H]⁺.

### Example 108

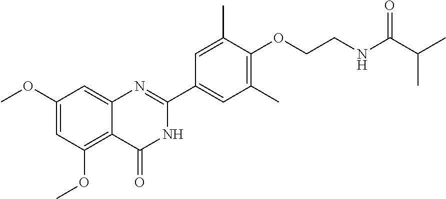 [SMILES: COc1cc(OC)c2c(=O)[nH]c(-c3cc(C)c(OCCNC(=O)C(C)C)c(C)c3)nc2c1]

### N-(2-(4-(5,7-dimethoxy-4-oxo-3,4-dihydroquinazolin-2-yl)-2,6-dimethylphenoxy)ethyl)isobutyramide

Following the methodology described for Example 107, the title compound was made from 2-(4-(2-aminoethoxy)-3,5-dimethylphenyl)-5,7-dimethoxyquinazolin-4(3H)-one in 59% yield and isolated as a white solid: MS (APCI) m/z 440 [M+H]⁺.

### Example 109

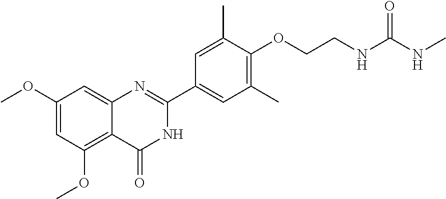

### 1-(2-(4-(5,7-dimethoxy-4-oxo-3,4-dihydroquinazolin-2-yl)-2,6-dimethylphenoxy)ethyl)-3-methylurea

A mixture of compound 2-(4-(2-aminoethoxy)-3,5-dimethylphenyl)-5,7-dimethoxyquinazolin-4(3H)-one (0.10 g, 0.27 mmol), methylisocyanate (0.020 g, 0.35 mmol), and Et₃N (0.034 g, 0.35 mmol) in THF (4.0 mL) was stirred at room temperature for 16 hours. The mixture was filtered, washed with CH₂Cl₂ and the solvent was removed under reduced pressure. The resulting residue was chromatographed on silica gel to yield the title compound (0.082 g, 71%) as a white solid: MS (APCI) m/z 449 [M+Na]⁺.

### Example 110

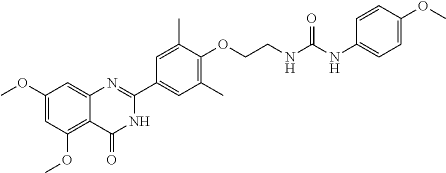

### 1-(2-(4-(5,7-dimethoxy-4-oxo-3,4-dihydroquinazolin-2-yl)-2,6-dimethylphenoxy)ethyl)-3-(4-methoxyphenyl)urea

Following the methodology described for Example 115, the title compound was made from 2-(4-(2-aminoethoxy)-3,5-dimethylphenyl)-5,7-dimethoxyquinazolin-4(3H)-one in 57% yield and isolated as a white solid: MS (APCI) m/z 541 [M+Na]⁺.

### Example 111

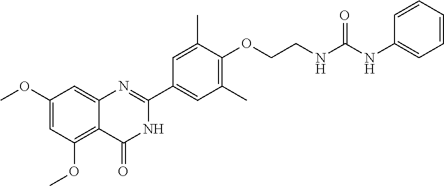 [SMILES: COc1cc(OC)c2c(=O)[nH]c(-c3cc(C)c(OCCNC(=O)Nc4ccccc4)c(C)c3)nc2c1]

### 1-(2-(4-(5,7-dimethoxy-4-oxo-3,4-dihydroquinazolin-2-yl)-2,6-dimethylphenoxy)ethyl)-3-phenylurea

Following the methodology described for Example 115, the title compound was made from 2-(4-(2-aminoethoxy)-3,5-dimethylphenyl)-5,7-dimethoxyquinazolin-4(3H)-one in 59% yield and isolated as a light yellow solid: MS (APCI) m/z 489 [M+H]⁺.

### Example 112

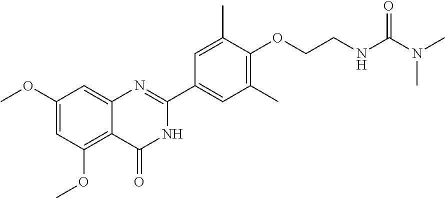

### 3-(2-(4-(5,7-dimethoxy-4-oxo-3,4-dihydroquinazolin-2-yl)-2,6-dimethylphenoxy)ethyl)-1,1-dimethylurea

Following the methodology described for Example 115, the title compound was made from 2-(4-(2-aminoethoxy)-3,5-dimethylphenyl)-5,7-dimethoxyquinazolin-4(3H)-one in 59% yield and isolated as a white solid: MS (APCI) m/z 441 [M+H]⁺.

### Example 113

### Inhibition of tetra-acetylated Histone H4 Binding Individual Bromodomains

Proteins were cloned and overexpressed with a N-terminal 6×His tag, then purified by nickel affinity followed by size exclusion chromatography. Briefly, E. coli BL21(DE3) cells were transformed with a recombinant expression vector encoding N-terminally Nickel affinity tagged bromodomains from Brd2, Brd3, or Brd4. Cell cultures were incubated at 37° C. with shaking to the appropriate density and induced overnight with IPTG. The supernatant of lysed cells was loaded onto Ni-IDA column for purification. Eluted protein was pooled, concentrated and further purified by size exclusion chromatography. Fractions representing monomeric protein were pooled, concentrated, aliquoted, and frozen at −80° C. for use in subsequent experiments.

Binding of tetra-acetylated histone H4 and BET bromodomains was confirmed by a Time Resolved Fluorescence Resonance Energy Transfer (TR-FRET) method. N-terminally His-tagged bromodomains (200 nM) and biotinylated tetra-acetylated histone H4 peptide (25-50 nM, Millipore) were incubated in the presence of Europium Cryptate-labeled streptavidin (Cisbio Cat. #610SAKLB) and XL665-labeled monoclonal anti-His antibody (Cisbio Cat. #61HISXLB) in a white 96 well microtiter plate (Greiner). For inhibition assays, serially diluted test compound was added to these reactions in a 0.2% final concentration of DMSO. Final buffer concentrations were 30 mM HEPES pH 7.4, 30 mM NaCl, 0.3 mM CHAPS, 20 mM phosphate pH 7.0, 320 mM KF, 0.08% BSA). After 2 hours incubation at room temperature, the fluorescence by FRET was measured at 665 and 620 nm by a SynergyH4 plate reader (Biotek). Illustrative results with the first bromodomain of Brd4 were shown in Table 2. The binding inhibitory activity was shown by a decrease in 665 nm fluorescence relative to 620 nm. IC₅₀ values were determined from a dose response curve. Compounds with an IC₅₀ value less than 50 uM were deemed to be active.

TABLE 2
Inhibition of Binding of Tetra-acetylated Histone H4
and Brd4 bromodomain 1 as Measured by FRET

| | FRET |
|---|---|
| | activity |
| Compound | (<50 uM) |
| 3-(4-Hydroxyphenyl)-2H-isoquinolin-1-one (Example 1) | Active |
| 4-(1-Oxo-1,2-dihydroisoquinolin-3-yl)phenyl 2-amino-5- | Active |
| guanidinopentanoate (Example 2) | |
| 3-(4-hydroxyphenyl)-6,8-dimethoxyisoquinolin-1(2H)-one | Active |
| (Example 3) | |
| 3-(4-hydroxy-3,5-dimethylphenyl)-6,8-dimethoxy-2- | Active |
| methylisoquinolin-1(2H)-one (Example 4) | |
| 7-(4-hydroxy-3,5-dimethylphenyl)-1,6-naphthyridin-5(6H)- | Inactive |
| one (Example 5) | |
| 3-(4-(2-hydroxyethoxy)-3,5-dimethylphenyl)-6,8- | Active |
| dimethoxyisoquinolin-1(2H)-one (Example 6) | |
| 3-(4-(2-(dimethylamino)ethoxy)-3,5-dimethylphenyl)-6,8- | Active |
| dimethoxyisoquinolin-1(2H)-one (Example 7) | |
| 2-hydroxy-7-(4-hydroxy-3,5-dimethylphenyl)-4-methoxy- | Inactive |
| 1,6-naphthyridin-5(6H)-one (Example 9) | |
| 2-(4-hydroxy-3,5-dimethylphenyl)-5,7- | Active |
| dimethoxyquinazolin-4(3H)-one (Example 10) | |
| 3-(4-(2-hydroxy-2-methylpropoxy)-3,5-dimethylphenyl)- | Active |
| 6,8-dimethoxyisoquinolin-1(2H)-one (Example 11) | |
| 6,8-dimethoxy-3-(4-hydroxy-3,5-dimethylphenyl)-2H-1,2- | Active |
| benzothiazine-1,1-dioxide (Example 12) | |
| 7-(4-hydroxy-3,5-dimethylphenyl)-2,4-dimethoxy-1,6- | Active |
| naphthyridin-5(6H)-one (Example 13) | |
| 3-(4-hydroxy-3,5-dimethylphenyl)-6,8-dimethoxy-2-methyl- | Inactive |
| 7-(morpholinomethyl) isoquinolin-1(2H)-one (Example 14) | |
| 3-(3,5-dimethyl-4-(2-morpholinoethoxy)phenyl)-6,8- | Active |
| dimethoxyisoquinolin-1(2H)-one (Example 15) | |
| 2-(4-(2-hydroxyethoxy)-3,5-dimethylphenyl)-5,7- | Active |
| dimethoxyquinazolin-4(3H)-one (Example 16) | |
| 2-(3,5-dimethyl-4-(2-morpholinoethoxy)phenyl)-5,7- | Active |
| dimethoxyquinazolin-4(3H)-one (Example 17) | |
| 3-(3,5-dimethyl-4-(2-(4-methylpiperazin-1- | Active |
| yl)ethoxy)phenyl)-6,8-dimethoxyisoquinolin-1(2H)-one | |
| (Example 18) | |
| 2-(4-hydroxyphenyl)-5,7-dimethoxyquinazolin-4(3H)-one | Active |
| (Example 19) | |
| 2-(4-(5,7-dimethoxy-4-oxo-3,4-dihydroquinazolin-2- | Active |
| yl)phenoxy)acetic acid (Example 20) | |
| 5,7-dimethoxy-2-(pyridin-2-yl)quinazolin-4(3H)-one | Active |
| (Example 21) | |
| 5,7-dimethoxy-2-(pyridin-3-yl)quinazolin-4(3H)-one | Active |
| (Example 22) | |
| 2-(3,5-di-tert-butyl-4-hydroxyphenyl)-5,7- | Inactive |
| dimethoxyquinazolin-4(3H)-one (Example 23) | |
| 2-(3,5-dimethoxyphenyl)-5,7-dimethoxyquinazolin-4(3H)- | Inactive |
| one (Example 24) | |
| 5,7-dimethoxy-2-(4-methoxyphenyl)quinazolin-4(3H)-one | Inactive |
| (Example 25) | |
| 2-(4-hydroxy-3-methoxyphenyl)-5,7-dimethoxyquinazolin- | Active |
| 4(3H)-one (Example 26) | |
| 2-(3-chloro-4-hydroxyphenyl)-5,7-dimethoxyquinazolin- | Active |
| 4(3H)-one (Example 27) | |
| 5,7-dimethoxy-2-(pyridin-4-yl)quinazolin-4(3H)-one | Inactive |
| (Example 28) | |
| 2-(3,5-dimethyl-4-(2-morpholinoethoxy)phenyl)quinazolin- | Inactive |
| 4(3H)-one (Example 29) | |
| 2-(4-(2-hydroxyethoxy)-3,5-dimethylphenyl)quinazolin- | Inactive |
| 4(3H)-one (Example 30) | |
| 2-(2,3-dihydrobenzo[b][1,4]dioxin-6-yl)quinazolin-4(3H)- | Inactive |
| one (Example 31) | |
| 2-(4-(dimethylamino)naphthalen-1-yl)-5,7- | Inactive |
| dimethoxyquinazolin-4(3H)-one (Example 32) | |
| 2-(4-(5,7-dimethoxy-4-oxo-3,4-dihydroquinazolin-2- | Active |
| yl)phenoxy)acetamide (Example 33) | |
| 2-(4-(4-oxo-3,4-dihydroquinazolin-2-yl)phenoxy)acetic | Inactive |
| acid (Example 34) | |
| 2-(4-(dimethylamino)naphthalen-1-yl)quinazolin-4(3H)-one | Inactive |
| (Example 35) | |
| 2-(4-(4-oxo-3,4-dihydroquinazolin-2-yl)phenoxy)acetamide | Inactive |
| (Example 36) | |
| 2-(2,3-dihydrobenzo[b][1,4]dioxin-6-yl)-5,7- | Active |
| dimethoxyquinazolin-4(3H)-one (Example 37) | |
| 5,7-dimethoxy-2-(4-(4-methylpiperazin-1- | Active |
| yl)phenyl)quinazolin-4(3H)-one (Example 38) | |
| 2-(4-(dimethylamino)pyridin-1-yl)-6,7- | Inactive |
| dimethoxyquinazolin-4(3H)-one (Example 39) | |
| 2-(4-(bis(2-hydroxyethyl)amino)phenyl)quinazolin-4(3H)- | Inactive |
| one (Example 40) | |
| 2-(4-(bis(2-hydroxyethyl)amino)phenyl)-5,7- | Active |
| dimethoxyquinazolin-4(3H)-one (Example 41) | |
| 2-(2,3-dihydrobenzo[b][1,4]dioxin-6-yl)-6,7- | Inactive |
| dimethoxyquinazolin-4(3H)-one (Example 43) | |
| 5,7-dimethoxy-2-(4-morpholinophenyl)quinazolin-4(3H)- | Active |
| one (Example 44) | |
| 7-(3,5-dimethyl-4-(2-morpholinoethoxy)phenyl)-2,4- | Active |
| dimethoxy-1,6-naphthyridin-5(6H)-one (Example 45) | |
| 3-(4-hydroxy-3,5-dimethylphenyl)-6,8-dimethoxy-7- | Active |
| (morpholinomethyl)isoquinolin-1(2H)-one (Example 46) | |
| 2-(4-hydroxy-3,5-dimethylphenyl)-6,7- | Active |
| dimethoxyquinazolin-4(3H)-one (Example 47) | |
| 3-(4-(5,7-dimethoxy-4-oxo-3,4-dihydroquinazolin-2- | Active |
| yl)phenyl)propanoic acid (Example 48) | |
| N-(2-(4-hydroxy-3,5-dimethylphenyl)-4-oxo-3,4- | Inactive |
| dihydroquinazolin-6-yl)acetamide (Example 49) | |
| 2-(4-(6,7-dimethoxy-4-oxo-3,4-dihydroquinazolin-2-yl)-2,6- | Inactive |
| dimethylphenoxy)acetamide (Example 50) | |
| 2-(3-chloro-4-(2-hydroxyethoxy)phenyl)-5,7- | Inactive |
| dimethoxyquinazolin-4(3H)-one (Example 51) | |
| 2-(4-(2-hydroxyethoxy)-3,5-dimethylphenyl)-6,7- | Inactive |
| dimethoxyquinazolin-4(3H)-one (Example 53) | |
| 5,7-dimethoxy-2-(4-((4-methylpiperazin-1- | Active |
| yl)methyl)phenyl)quinazolin-4(3H)-one (Example 54) | |
| N-(4-(5,7-dimethoxy-4-oxo-3,4-dihydroquinazolin-2-yl)- | Active |
| 2,6-dimethylphenyl)-2-hydroxyacetamide (Example 55) | |
| 2-(4-hydroxy-3,5-dimethylphenyl)-6-(morpholinomethyl) | Active |
| quinazolin-4(3H)-one (Example 57) | |
| 2,4-dimethoxy-7-(4-methoxy-3,5-dimethylphenyl)-1,6- | Active |
| naphthyridin-5(6H)-one (Example 58) | |
| 2-(4-(5,7-dimethoxy-4-oxo-3,4-dihydroquinazolin-2-yl)-2,6- | Active |
| dimethylphenoxy)acetic acid (Example 59) | |
| N-(4-(5,7-dimethoxy-4-oxo-3,4-dihydroquinazolin-2- | Inactive |
| yl)phenyl)-2-hydroxyacetamide (Example 60) | |
| 5,7-dimethoxy-2-(4-(morpholinomethyl)phenyl)quinazolin- | Active |
| 4(3H)-one (Example 61) | |
| 2-(4-((4-ethylpiperazin-1-yl)methyl)phenyl)-5,7- | Active |
| dimethoxyquinazolin-4(3H)-one (Example 62) | |
| 2-(4-hydroxy-3,5-dimethylphenyl)-5,7- | Active |
| dimethoxypyrido[2,3-d]pyrimidin-4(3H)-one (Example 63) | |
| 5,7-dimethoxy-2-(4-methoxy-3-(morpholinomethyl)phenyl) | Active |
| quinazolin-4(3H)-one (Example 64) | |
| 2-(4-(2-hydroxyethoxy)-3,5-dimethylphenyl)-5,7- | Active |
| dimethoxypyrido[2,3-d]pyrimidin-4(3H)-one (Example 65) | |
| 2-(4-hydroxy-3,5-dimethylphenyl)-5,7-dimethoxy-1- | Active |
| methylquinazolin-4(1H)-one (Example 66) | |
| 2-(3,5-dimethyl-4-(2-(pyrrolidin-1-yl)ethoxy)phenyl)-5,7- | Active |
| dimethoxyquinazolin-4(3H)-one (Example 67) | |
| N-(2-(4-(2-hydroxyethoxy)-3,5-dimethylphenyl)-4-oxo-3,4- | Inactive |
| dihydroquinazolin-6-yl)acetamide (Example 68) | |
| 7-(4-hydroxy-3,5-dimethylphenyl)-2,4-diisopropoxy-1,6- | Active |
| naphthyridin-5(6H)-one (Example 69) | |
| 2-(4-hydroxy-3-(2-hydroxyethyl)phenyl)-5,7- | Active |
| dimethoxyquinazolin-4(3H)-one (Example 70) | |
| 2-(4-(2-hydroxyethoxy)-3,5-dimethylphenyl)-5,7- | Inactive |
| dimethylquinazolin-4(3H)-one (Example 71) | |
| 2-(4-(bis(2-hydroxyethyl)amino)phenyl)-5,7-dimethoxy- | Active |
| pyrido[2,3-d]pyrimidin-4(3H)one (Example 72) | |
| 5,7-dimethoxy-2-(4-(2-methoxyethoxy)-3,5- | Active |
| dimethylphenyl)quinazolin-4(3H)-one (Example 73) | |
| 5,7-dichloro-2-(4-(2-hydroxyethoxy)-3,5- | Active |
| dimethylphenyl)quinazolin-4(3H)-one (Example 74) | |
| 2-(4-hydroxy-3,5-dimethylphenyl)-5,7-dimethoxy-6- | Active |
| (morpholinomethyl)quinazolin-4(3H)-one (Example 75) | |
| 2-(4-(2-hydroxyethoxy)-3,5-dimethylphenyl)-6- | Inactive |
| methoxyquinazolin-4(3H)-one (Example 76) | |
| 2-(2-chlorophenyl)-5,7-dimethoxyquinazolin-4(3H)-one | Inactive |
| (Example 77) | |
| 2-(4-(2-hydroxyethoxy)-3,5-dimethylphenyl)-5- | Active |
| methoxyquinazolin-4(3H)-one (Example 78) | |
| 5,7-dimethoxy-2-(6-(4-(methylsulfonyl)phenyl)pyridin-2- | Active |
| yl)quinazolin-4(3H)-one (Example 80) | |
| 5,7-dimethoxy-2-(6-methylpyridin-2-yl)quinazolin-4(3H)- | Active |
| one (Example 81) | |
| 5,7-dimethoxy-2-(6-(4-(methylthio)phenyl)pyridin-2-yl) | Inactive |
| quinazolin-4(3H)-one (Example 82) | |
| 2-(2-chloro-6-methylpyridin-4-yl)-5,7-dimethoxyquinazolin- | Inactive |
| 4(3H)-one (Example 83) | |
| 5,7-dimethoxy-2-(4-methoxy-3,5-dimethylphenyl) | Active |
| quinazolin-4(3H)-one (Example 84) | |
| 2-(4-amino-3,5-dimethylphenyl)-5,7-dimethoxyquinazolin- | Active |
| 4(3H)-one (Example 85) | |
| 2-(3,5-dimethylphenyl)-5,7-dimethoxyquinazolin-4(3H)- | Active |
| one (Example 86) | |
| (E)—N′-(4-(5,7-dimethoxy-4-oxo-3,4-dihydroquinazolin-2- | Active |
| yl)-2,6-dimethylphenyl)-N,N-dimethylformimidamide | |
| (Example 87) | |
| 6-bromo-2-(4-hydroxy-3,5-dimethylphenyl)quinazolin- | Inactive |
| 4(3H)-one (Example 88) | |
| 6-bromo-2-(4-(2-hydroxyethoxy)-3,5-dimethylphenyl) | Inactive |
| quinazolin-4(3H)-one (Example 89) | |
| 2-(4-(benzyloxy)-3,5-dimethylphenyl)-5,7- | Inactive |
| dimethoxyquinazolin-4(3H)-one (Example 90) | |
| 2-(4-(2-hydroxyethoxy)-3-methylphenyl)-5,7- | Active |
| dimethoxyquinazolin-4(3H)-one (Example 91) | |
| 2-(4-hydroxy-3-methylphenyl)-5,7-dimethoxyquinazolin- | Active |
| 4(3H)-one (Example 92) | |
| N1-(2-(4-(5,7-dimethoxy-4-oxo-3,4-dihydroquinazolin-2- | Active |
| yl)-2,6-dimethylphenoxy)ethyl)-N2-methylphthalamide | |
| (Example 93) | |
| N-(2-(4-(5,7-dimethoxy-4-oxo-3,4-dihydroquinazolin-2-yl)- | Inactive |
| 2,6-dimethylphenoxy)ethyl)-4-methoxybenzene | |
| sulfonamide (Example 94) | |
| 4-chloro-N-(2-(4-(5,7-dimethoxy-4-oxo-3,4- | Inactive |
| dihydroquinazolin-2-yl)-2,6-dimethylphenoxy)ethyl) | |
| benzenesulfonamide (Example 95) | |
| N-(2-(4-(5,7-dimethoxy-4-oxo-3,4-dihydroquinazolin-2-yl)- | Active |
| 2,6-dimethylphenoxy)ethyl)methanesulfonamide | |
| (Example 96) | |
| 2-(4-(5,7-dimethoxy-4-oxo-3,4-dihydroquinazolin-2-yl)-2- | Active |
| methoxyphenoxy)acetic acid (Example 97) | |
| 5-hydroxy-2-(4-hydroxy-3,5-dimethylphenyl)-7- | Inactive |
| methoxyquinazolin-4(3H)-one (Example 98) | |
| 2-(4-(5,7-Dimethoxy-4-oxo-3,4-dihydroquinazolin-2-yl)- | Active |
| 2,6-dimethyl-phenoxy)ethyl propylcarbamate | |
| (Example 99) | |
| 2-(4-(5,7-Dimethoxy-4-oxo-3,4-dihydroquinazolin-2-yl)- | Active |
| 2,6-dimethyl-phenoxy)ethyl methylcarbamate | |
| (Example 100) | |
| N-(2-(4-(5,7-Dimethoxy-4-oxo-3,4-dihydroquinazolin-2-yl)- | Active |
| 2,6-dimethylphenoxy)ethyl)-4-methylbenzamide | |
| (Example 101) | |
| 2-(4-(5,7-dimethoxy-4-oxo-3,4-dihydroquinazolin-2-yl)-2,6- | Active |
| dimethylphenoxy)ethyl cyclohexylcarbamate | |
| (Example 102) | |
| N-(2-(4-(5,7-dimethoxy-4-oxo-3,4-dihydroquinazolin-2-yl)- | Inactive |
| 2,6-dimethylphenoxy)ethyl)benzenesulfonamide | |
| (Example 103) | |
| N-(2-(4-(5,7-dimethoxy-4-oxo-3,4-dihydroquinazolin-2-yl)- | Inactive |
| 2,6-dimethylphenoxy)ethyl)-4-methylbenzenesulfonamide | |
| (Example 104) | |
| N-(2-(4-(5,7-dimethoxy-4-oxo-3,4-dihydroquinazolin-2-yl)- | Inactive |
| 2,6-dimethylphenoxy)ethyl)-4-methoxybenzamide | |
| (Example 105) | |
| N-(2-(4-(5,7-dimethoxy-4-oxo-3,4-dihydroquinazolin-2-yl)- | Active |
| 2,6-dimethylphenoxy)ethyl)acetamide (Example 106) | |
| N-(2-(4-(5,7-dimethoxy-4-oxo-3,4-dihydroquinazolin-2-yl)- | Active |
| 2,6-dimethylphenoxy)ethyl)benzamide (Example 107) | |
| N-(2-(4-(5,7-dimethoxy-4-oxo-3,4-dihydroquinazolin-2-yl)- | Active |
| 2,6-dimethylphenoxy)ethyl)isobutyramide (Example 108) | |
| 1-(2-(4-(5,7-dimethoxy-4-oxo-3,4-dihydroquinazolin-2-yl)- | Active |
| 2,6-dimethylphenoxy)ethyl)-3-methylurea (Example 109) | |
| 1-(2-(4-(5,7-dimethoxy-4-oxo-3,4-dihydroquinazolin-2-yl)- | Inactive |
| 2,6-dimethylphenoxy)ethyl)-3-(4-methoxyphenyl)urea | |
| (Example 110) | |
| 1-(2-(4-(5,7-dimethoxy-4-oxo-3,4-dihydroquinazolin-2-yl)- | Active |
| 2,6-dimethylphenoxy)ethyl)-3-phenylurea (Example 111) | |
| 3-(2-(4-(5,7-dimethoxy-4-oxo-3,4-dihydroquinazolin-2-yl)- | Active |
| 2,6-dimethylphenoxy)ethyl)-1,1-dimethylurea | |
| (Example 112) | |

### Example 114

### Inhibition of c-Myc Expression in Cancer Cell Lines

MV4-11 cells (2.5×10⁴ cells) were plated in 96 well U-bottom plates with test compound or DMSO (0.1%), and incubated for 3 hours at 37° C. Cells were then harvested by centrifugation, lysed, and mRNA was isolated using the mRNA catcher plus kit (Invitrogen). Reverse transcription of the mRNA and duplex amplification of the c-myc and cyclophilin cDNAs were performed using the RNA Ultrasense kit (Invitrogen) and a ViiA7 real-time PCR machine (Applied Biosystems). IC₅₀ values were determined from a dose response curve. Compounds with an IC₅₀ value less than 30 μM were deemed to be active (Table 3).

TABLE 3
Inhibition of c-myc Activity in Human AML MV4-11 cells

| | c-myc |
|---|---|
| | activity |
| Compound | (<30 uM) |
| 3-(4-hydroxy-3,5-dimethylphenyl)-6,8-dimethoxy-2- | Active |
| methylisoquinolin-1(2H)-one (Example 4) | |
| 2-(4-hydroxy-3,5-dimethylphenyl)-5,7-dimethoxyquinazolin- | Active |
| 4(3H)-one (Example 10) | |
| 2-(4-(2-hydroxyethoxy)-3,5-dimethylphenyl)-5,7- | Active |
| dimethoxyquinazolin-4(3H)-one (Example 16) | |
| 5,7-dimethoxy-2-(4-(4-methylpiperazin-1- | Active |
| yl)phenyl)quinazolin-4(3H)-one (Example 38) | |
| 3-(4-hydroxy-3,5-dimethylphenyl)-6,8-dimethoxy-7- | Active |
| (morpholinomethyl)isoquinolin-1(2H)-one (Example 46) | |
| 2-(4-hydroxy-3,5-dimethylphenyl)-6- | Active |
| (morpholinomethyl)quinazolin-4(3H)-one (Example 57) | |
| 2-(4-(5,7-dimethoxy-4-oxo-3,4-dihydroquinazolin-2-yl)-2,6- | Inactive |
| dimethylphenoxy)acetic acid (Example 59) | |
| 2-(3,5-dimethyl-4-(2-(pyrrolidin-1-yl)ethoxy)phenyl)-5,7- | Active |
| dimethoxyquinazolin-4(3H)-one (Example 67) | |
| 2-(4-hydroxy-3,5-dimethylphenyl)-5,7-dimethoxy-6- | Active |
| (morpholinomethyl)quinazolin-4(3H)-one (Example 75) | |
| N-(2-(4-(5,7-dimethoxy-4-oxo-3,4-dihydroquinazolin-2-yl)-2, | Inactive |
| 6-dimethylphenoxy)ethyl)methanesulfonamide (Example 96) | |

### Example 115

### Inhibition of Cell Proliferation in Human AML MV-4-11 Cells

MV4-11 cells: 96-well plates were seeded with 5×10⁴ cells per well of exponentially growing human AML MV-4-11 (CRL-9591) cells and immediately treated with two-fold dilutions of test compounds, ranging from 30 uM to 0.2 μM. Triplicate wells were used for each concentration, as well as a media only and three DMSO control wells. The cells and compounds were incubated at 37° C., 5% CO₂ for 72 hours before adding 20 μL of the CellTiter Aqueous One Solution (Promega) to each well and incubating at 37° C., 5% CO₂ for an additional 3-4 hours. The absorbance was taken at 490 nm in a spectrophotometer and the percentage of proliferation relative to DMSO-treated cells was calculated after correction from the blank well. IC₅₀ were calculated using the GraphPad Prism software. Compounds with an IC₅₀ value less than 30 uM were deemed to be active (Table 4).

TABLE 4
Inhibition of Cell Proliferation in Human AML MV-4-11 cells

| | cell |
|---|---|
| | proliferation |
| | activity |
| Compound | (<30 uM) |
| 3-(4-hydroxy-3,5-dimethylphenyl)-6,8-dimethoxy-2- | Active |
| methylisoquinolin-1(2H)-one (Example 4) | |
| 2-(4-hydroxy-3,5-dimethylphenyl)-5,7- | Active |
| dimethoxyquinazolin-4(3H)-one (Example 10) | |
| 2-(4-(2-hydroxyethoxy)-3,5-dimethylphenyl)-5,7- | Active |
| dimethoxyquinazolin-4(3H)-one (Example 17) | |
| 5,7-dimethoxy-2-(4-(4-methylpiperazin-1- | Active |
| yl)phenyl)quinazolin-4(3H)-one (Example 38) | |
| 3-(4-hydroxy-3,5-dimethylphenyl)-6,8-dimethoxy-7- | Active |
| (morpholinomethyl)isoquinolin-1(2H)-one (Example 46) | |
| 2-(4-hydroxy-3,5-dimethylphenyl)-6- | Active |
| (morpholinomethyl)quinazolin-4(3H)-one (Example 57) | |
| 2-(3,5-dimethyl-4-(2-(pyrrolidin-1-yl)ethoxy)phenyl)-5,7- | Active |
| dimethoxyquinazolin-4(3H)-one (Example 67) | |
| 2-(4-hydroxy-3,5-dimethylphenyl)-5,7-dimethoxy-6- | Active |
| (morpholinomethyl)quinazolin-4(3H)-one (Example 75) | |
| N-(2-(4-(5,7-dimethoxy-4-oxo-3,4-dihydroquinazolin-2-yl)- | Active |
| 2,6-dimethylphenoxy)ethyl)methanesulfonamide | |
| (Example 96) | |

### Example 116

### Lipopolysaccharide (LPS) Stimulated Whole Blood Assay for Measuring TNFa and IL-6 Levels

Activation of monocytic cells by agonists of toll-like receptors such as bacterial lipopolysaccharide (LPS) results in production of key inflammatory mediators including IL-6 and TNFa. Such pathways are widely considered to be central to the pathophysiology of a range of auto-immune and inflammatory disorders. Compounds to be tested are diluted to give a range of appropriate concentrations and 1 μl of the dilution stocks is added to wells of a 96 plate. Following addition of whole blood (130 μL) the plates are incubated at 37 degrees (5% CO₂) for 30 min before the addition of 10 μl of 2.8 μg/mL lipopolysaccharides (LPS), diluted in complete RPMI 1640 (final concentration=200 ng/mL), to give a total volume of 140 μL per well. After further incubation for 24 hours at 37 degrees, 140 μL of PBS are added to each well. The plates are sealed, shaken for 10 minutes and then centrifuged (2500 rpm×10 min). 100 μL of the supernatant are removed and IL-6 and TNFa levels assayed by immunoassay (typically by MesoScale Discovery technology) either immediately or following storage at −20 degrees. BET inhibitors tested in this assay will inhibit the production of the key inflammatory mediator IL-6 and/or TNFa.

### Example 117

### In Vivo Mouse Endotoxemia Model Assay

High doses of Endotoxin (bacterial lipopolysaccharide) are administered to animals produce a profound shock syndrome including a strong inflammatory response, dysregulation of cardiovascular function, organ failure and ultimately mortality. This pattern of response is very similar to human sepsis and septic shock, where the body's response to a significant bacterial infection can be similarly life threatening. To test the compounds for use in the invention groups of Balb/c male mice are given a lethal dose of 15 mg/kg LPS by intraperitoneal injection. Ninety minutes later, animals are dosed intravenously with vehicle (20% cyclodextrin 1% ethanol in apyrogen water) or test compound (10 mg/kg). The survival of animals is evaluated at 4 days. BET inhibitors tested in the mouse endotoxemia model assay will result in a significant animal survival effect following intravenous administration.

### Example 118

### Growth Suppressive Activity Test Against Cancer Cells

Using RPMI 1640 medium (manufactured by SIGMA) supplemented with 10% fetal bovine serum, human promyelocytic leukemia-derived cell line HL-60, human acute lymphoblastic leukemia-derived cell line MOLT4, human Burkitt's lymphoma-derived cell line Daudi, and human multiple myeloma-derived cell line RPMI-8226 are each cultured at 37° C., 5% CO₂. In addition, using ISKOV medium (manufactured by SIGMA) supplemented with 10% fetal bovine serum, human chronic myeloid leukemia-derived cell line MV4-11 is cultured at 37° C., 5% CO₂. Moreover, using DMEM/F-12 medium (manufactured by SIGMA) supplemented with 10% fetal bovine serum, human lung cancer cell-derived cell line EBC-1, human hepatocellular cancer-derived cell line Kim-1, human colorectal cancer-derived cell line HCT-116, human prostate cancer-derived cell line PC-3, human ovarian cancer-derived cell line A2780, and human osteosarcoma-derived cell line Saos2 are each cultured at 37° C., 5% CO₂. These cells are plated on a 96 well plate, and cultured for 1 day. To each culture test compound diluted with the medium to a final concentration of 0.0003⁻¹⁰ μm (final DMSO concentration, 0.4%) is added. After culture for 3 more days, WST-8 (0.16 mg/mL) is added to the culture medium and the cells are cultured for 2 hr. The absorbance at 650 nm is subtracted from the absorbance at 450 nm. The growth suppressive activity is shown by a decrease rate of the absorbance of the group receiving test compound to that of the control group, and GI₅₀ value is determined from a dose-reaction curve plotting a decrease rate of the absorbance obtained by changing the compound concentrations.

This assay demonstrates that a compound that inhibits binding between acetylated histone, more specifically acetylated histone H4, and a bromodomain-containing protein, more specifically human-derived BET family protein BRD2, BRD3 or BRD4 can be used as an antitumor agent.

### Example 119

### HIV Tat-Mediated Transactivation Inhibition Assay

This assay evaluates inhibition of Tat-mediated transactivation by BET inhibitors that block the PCAF bromodomain interaction with HIV-1 Tat-AcK50. The effect is assessed by a microinjection study as described previously by Dorr et al. (EMBO J. 21; 2715-2723, 2002). In this microinjection assay, HeLa-Tat cells are grown on Cellocate coverslips and microinjected at room temperature with an automated injection system (Carl Zeiss). Samples are prepared as a 20 μl injection mix containing the LTR-luciferase (100 ng/ml) and CMV-GFP (50 ng/ml) constructs together with 5 mg/ml a chemical compound or pre-immune IgGs. Live cells are examined on a Zeiss Axiovert microscope to determine the number of GFP-positive cells. Four hours after injection, cells are washed in cold phosphate buffer and processed for luciferase assays (Promega). BET inhibitors tested in this assay will inhibit Tat-mediated transactivation by the PCAF BRD inhibitor.

### Example 120

### Whole Blood Assay IL6 ELISA

Whole, fresh, heparinized blood was collected and diluted 1× in RPMI media+compounds and DMSO, in 1 mL volume total. Samples are incubated on a rotator, in the TC incubator, and treated for 1 h with compound and 3 h with 1 ug/mL LPS. Serum is harvested for ELISA analysis, and then RBCs are lysed with ammonium chloride, and lymphocytes are collected. Media is then harvested and ELISAs performed. The above experiment is performed in duplicate.

All references referred to herein are incorporated by reference in their entirety. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for inhibiting BET proteins comprising administering to a mammal, such as a human, a therapeutically effective amount of a compound of Formula I: 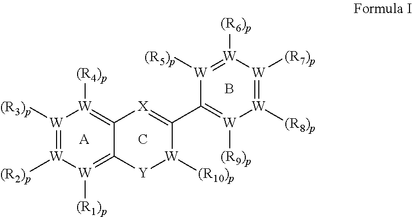
wherein:
X is selected from CR₁₁ and N;
Y is selected from CO and SO₂;
R₁, R₂, R₃, R₄, R₅, R₆, R₇, R₈, R₉, R₁₀ and R₁₁, are each independently selected from alkoxy, aryloxy, alkyl, amide, amino, aryl, arylalkyl, carbamate, carboxy, cycloalkyl, halogen, haloalkyl, heteroaryl, heterocyclyl, hydrogen, hydroxyl, ketone, phosphate, sulfide, sulfinyl, sulfonyl, sulfonic acid, sulfonamide and thioketone; and
each W is independently selected from C and N, wherein if W is N, then p is 0 and if W is C, then p is 1;
or tautomer, stereoisomer, pharmaceutically acceptable salt, or hydrate thereof.

2. A method of inhibiting BET proteins in a mammal, such as a human, comprising administering a therapeutically effective amount of a compound of Formula II: 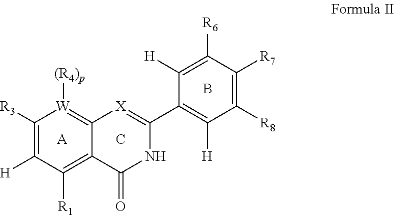
wherein:
X is selected from CH and N;
R₁ and R₃ are each independently selected from alkoxy, alkyl, and halogen;
R₄ is H;
R₆ and R₈ are each independently selected from alkoxy, alkyl, halogen, and hydrogen;
R₇ is selected from alkoxy, alkyl, amino, ether, hydrogen, and hydroxyl; and
W is selected from C and N, wherein if W is N, then p is 0 or 1, and if W is C, then p is 1;
or tautomer, stereoisomer, pharmaceutically acceptable salt, or hydrate thereof.

3. The method according to claim 1, wherein X is selected from CH and N.

4. The method according to claim 1, wherein the compound of Formula I is selected from:
3-(4-hydroxy-3,5-dimethylphenyl)-6,8-dimethoxy-7-(morpholinomethyl)isoquinolin-1(2H)-one;
2-(4-(bis(2-hydroxyethyl)amino)phenyl)-6,7-dimethoxyquinazolin-4(3H)-one;
2-(2,3-dihydrobenzo[b][1,4]dioxin-6-yl)-6,7-dimethoxyquinazolin-4(3H)-one;
2-(2-chloro-6-methylpyridin-4-yl)-5,7-dimethoxyquinazolin-4(3H)-one;
3-(4-Hydroxyphenyl)-2H-isoquinolin-1-one;
4-(1-Oxo-1,2-dihydroisoquinolin-3-yl)phenyl 2-amino-5-guanidino-pentanoate trihydrochloride;
3-(4-hydroxy-3,5-dimethylphenyl)-6,8-dimethoxy-2-methylisoquinolin-1(2H)-one;
3-(4-hydroxy-3,5-dimethylphenyl)-6,8-dimethoxy-2,7-dimethylisoquinolin-1(2H)-one;
3-(4-hydroxy-3,5-dimethylphenyl)-6,8-dimethoxy-2-methyl-7-(morpholino-methyl)isoquinolin-1(2H)-one;
5,7-dimethoxy-2-(pyridin-2-yl)quinazolin-4(3H)-one;
5,7-dimethoxy-2-(pyridin-3-yl)quinazolin-4(3H)-one;
5,7-dimethoxy-2-(pyridin-4-yl)quinazolin-4(3H)-one;
2-(3,5-dimethyl-4-(2-morpholinoethoxy)phenyl)quinazolin-4(3H)-one;
2-(4-(2-hydroxyethoxy)-3,5-dimethylphenyl)quinazolin-4(3H)-one;
2-(2,3-dihydrobenzo[b][1,4]dioxin-6-yl)quinazolin-4(3H)-one;
2-(4-(4-oxo-3,4-dihydroquinazolin-2-yl)phenoxy)acetic acid;
2-(4-(dimethylamino)pyridinon-1-yl)quinazolin-4(3H)-one;
2-(4-(4-oxo-3,4-dihydroquinazolin-2-yl)phenoxy)acetamide;
2-(4-(dimethylamino)pyridinon-1-yl)-6,7-dimethoxyquinazolin-4(3H)-one;
2-(4-(bis(2-hydroxyethyl)amino)phenyl)quinazolin-4(3H)-one;
2-(4-hydroxy-3,5-dimethylphenyl)-6,7-dimethoxyquinazolin-4(3H)-one;
N-(2-(4-hydroxy-3,5-dimethylphenyl)-4-oxo-3,4-dihydroquinazolin-6-yl)acetamide;
2-(4-(6,7-dimethoxy-4-oxo-3,4-dihydroquinazolin-2-yl)-2,6-dimethyl-phenoxy)acetamide;
2-(4-(2-hydroxyethoxy)-3,5-dimethylphenyl)-6,7-dimethoxyquinazolin-4(3H)-one;
2-(4-hydroxy-3,5-dimethylphenyl)-6-(morpholinomethyl)quinazolin-4(3H)-one;
N-(2-(4-(2-hydroxyethoxy)-3,5-dimethylphenyl)-4-oxo-3,4-dihydro-quinazolin-6-yl)acetamide;
2-(4-hydroxy-3,5-dimethylphenyl)-5,7-dimethoxy-6-(morpholino-methyl)quinazolin-4(3H)-one;
2-(4-(2-hydroxyethoxy)-3,5-dimethylphenyl)-6-methoxyquinazolin-4(3H)-one;
2-(2-chlorophenyl)-5,7-dimethoxyquinazolin-4(3H)-one;
2-(4-(2-hydroxyethoxy)-3,5-dimethylphenyl)-5-methoxyquinazolin-4(3H)-one;
5,7-dimethoxy-2-o-tolylquinazolin-4(3H)-one;
5,7-dimethoxy-2-(6-(4-(methylsulfonyl)phenyl)pyridin-2-yl)quinazolin-4(3H)-one;
5,7-dimethoxy-2-(6-methylpyridin-2-yl)quinazolin-4(3H)-one;
5,7-dimethoxy-2-(6-(4-(methylthio)phenyl)pyridin-2-yl)quinazolin-4(3H)-one;
5,7-dimethoxy-2-(1-phenyl-5-propyl-1H-pyrazol-4-yl)quinazolin-4(3H)-one;
2-(3-(2,6-dichlorophenyl)-5-methylisoxazol-4-yl)-5,7-dimethoxyquinazolin-4(3H)-one;
6-bromo-2-(4-hydroxy-3,5-dimethylphenyl)quinazolin-4(3H)-one;
6-bromo-2-(4-(2-hydroxyethoxy)-3,5-dimethylphenyl)quinazolin-4(3H)-one;
6-bromo-2-(4-(2-(tert-butyldimethylsilyloxy)ethoxy)-3,5-dimethyl-phenyl)quinazolin-4(3H)-one; and
5-hydroxy-2-(4-hydroxy-3,5-dimethylphenyl)-7-methoxyquinazolin-4(3H)-one;
and stereoisomers, tautomers, pharmaceutically acceptable salts, and hydrates thereof.

5. The method according to claim 2, wherein R₁ and R₃ are each independently an alkoxy.

6. The method according to claim 5, wherein R₆ and R₈ are independently selected from alkyl and hydrogen; R₇ is selected from amino and alkoxy, and X is N.

7. The method according to claim 2, wherein at least two of R₆, R₇, and R₈ are not hydrogen.

8. The method according to claim 7, wherein R₆ and R₈ are each independently selected from alkyl and hydrogen; R₇ is selected from amino, alkoxy; and X is N.

9. The method according to claim 2, wherein R₇ is an alkoxy group substituted with an amine.

10. The method according to claim 2, wherein R₇ is an alkoxy substituted with a cyclic amine.

11. The method according to claim 10, wherein the compound of Formula II is 2-(3,5-dimethyl-4-(2-(pyrrolidin-1-yl)ethoxy)phenyl)-5,7-dimethoxyquinazolin-4(3H)-one.

12. The method according to claim 2, wherein R₇ is an amino or an alkoxy selected from the group represented by Formula 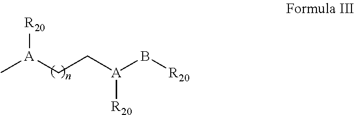
wherein:
A is selected from O and N;
n is selected from 0, 1, 2, and 3;
B is selected from —C(O)N(Rₕ)₂—, —S(O)₂N(Rₕ)₂—, —C(O)—, —S(O)₂—, and —C(O)O—, wherein each Rₕ is independently selected from alkyl, aryl, arylalkyl, cycloalkyl, haloalkyl, heteroaryl, heterocyclyl, and hydrogen; and
R₂₀ is selected from C₁-C₆ alkyl, C₁-C₆ alkenyl, C₁-C₆ alkynyl, aryl, arylalkyl, cycloalkyl, haloalkyl, heteroaryl, heterocyclyl, and hydrogen.

13. The method according to claim 2, wherein R₇ is selected from alkoxy, alkyl, amino, and hydroxyl.

14. The method according to claim 13, wherein:
X is N;
R₁ and R₃ are each independently selected from alkoxy;
R₆ and R₈ are each independently selected from alkyl, alkoxy, and hydrogen;
R₇ is selected from amino, alkoxy, and alkyl substituted with a heterocyclyl;
with the proviso that if R₇ is selected from alkoxy, then at least one of R₆ and R₈ is alkyl or alkoxy.

15. The method according to claim 2, wherein the compound of Formula II is selected from:
2-(4-(2-hydroxyethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethyl-phenoxy)ethyl)methanesulfonamide;
2-(4-hydroxy-3-methylphenyl)-5,7-dimethoxyquinazolin-4(3H)-one;
2-(4-(2-hydroxyethoxy)-3-methylphenyl)-5,7-dimethoxyquinazolin-4(3H)-one;
2-(4-(bis(2-hydroxyethyl)amino)phenyl)-5,7-dimethoxyquinazolin-4(3H)-one;
2-(3,5-dimethyl-4-(2-morpholinoethoxy)phenyl)-5,7-dimethoxyquinazolin-4(3H)-one;
2-(3,5-dimethyl-4-(2-(pyrrolidin-1-yl)ethoxy)phenyl)-5,7-dimethoxy-quinazolin-4(3H)-one;
2-(4-(5,7-dimethoxy-4-oxo-3,4-dihydroquinazolin-2-yl)-2,6-dimethyl-phenoxy)ethyl cyclohexylcarbamate;
N-(2-(4-(5,7-dimethoxy-4-oxo-3,4-dihydroquinazolin-2-yl)-2,6-dimethyl-phenoxy)ethyl)acetamide;
N-(2-(4-(5,7-dimethoxy-4-oxo-3,4-dihydroquinazolin-2-yl)-2,6-dimethyl-phenoxy)ethyl)isobutyramide;
1-(2-(4-(5,7-dimethoxy-4-oxo-3,4-dihydroquinazolin-2-yl)-2,6-dimethyl-phenoxy)ethyl)-3-phenylurea;
3-(2-(4-(5,7-dimethoxy-4-oxo-3,4-dihydroquinazolin-2-yl)-2,6-dimethyl-phenoxy)ethyl)-1,1-dimethylurea;
3-(4-hydroxy-3,5-dimethylphenyl)-6,8-dimethoxyisoquinolin-1(2H)-one;
3-(4-(2-hydroxyethoxy)-3,5-dimethylphenyl)-6,8-dimethoxyisoquinolin-1(2H)-one;
3-(4-hydroxy-3,5-dimethylphenyl)-7-(morpholinomethyl)isoquinolin-1(2H)-one;
2-(4-hydroxy-3,5-dimethylphenyl)-5,7-dimethoxyquinazolin-4(3H)-one;
3-(4-(2-hydroxy-2-methylpropoxy)-3,5-dimethylphenyl)-6,8-dimethoxyisoquinolin-1(2H)-one;
7-(4-hydroxy-3,5-dimethylphenyl)-2,4-dimethoxy-1,6-naphthyridin-5(6H)-one;
2-(4-(2-hydroxyethoxy)-3,5-dimethylphenyl)-5,7-dimethoxyquinazolin-4(3H)-one);
3-(3,5-dimethyl-4-(2-(4-methylpiperazin-1-yl)ethoxy)phenyl)-6,8-dimethoxyisoquinolin-1(2H)-one;
2-(4-hydroxy-3-methoxyphenyl)-5,7-dimethoxyquinazolin-4(3H)-one;
2-(4-(bis(2-hydroxyethyl)amino)phenyl)-5,7-dimethoxyquinazolin-4(3H)-one;
2-(4-((4-ethylpiperazin-1-yl)methyl)phenyl)-5,7-dimethoxyquinazolin-4(3H)-one;
2-(4-(2-hydroxyethoxy)-3,5-dimethyl phenyl)-5,7-dimethoxypyrido[2,3-d]pyrimidin-4(3H)-one;
5,7-dimethoxy-2-(4-methoxy-3,5-dimethylphenyl)quinazolin-4(3H)-one;
2-(4-amino-3,5-dimethylphenyl)-5,7-dimethoxyquinazolin-4(3H)-one;
N1-(2-(4-(5,7-dimethoxy-4-oxo-3,4-dihydroquinazolin-2-yl)-2,6-dimethylphenoxy)ethyl)-N2-methylphthalamide;
2-(4-(2-aminoethoxy)-3,5-dimethylphenyl)-5,7-dimethoxyquinazolin-4(3H)-one;
4-chloro-N-(2-(4-(5,7-dimethoxy-4-oxo-3,4-dihydroquinazolin-2-yl)-2,6-dimethylphenoxy)ethyl)benzenesulfonamide;
3-(4-hydroxyphenyl)-6,8-dimethoxyisoquinolin-1(2H)-one;
3-(4-hydroxy-3,5-dimethylphenyl)-6,8-dimethoxyisoquinolin-1(2H)-one;
7-(4-hydroxy-3,5-dimethylphenyl)-1,6-naphthyridin-5(6H)-one;
3-(4-(2-hydroxyethoxy)-3,5-dimethylphenyl)-6,8-dimethoxyisoquinolin-1(2H)-one;
3-(4-(2-(dimethylamino)ethoxy)-3,5-dimethylphenyl)-6,8-dimethoxy-isoquinolin-1(2H)-one;
3-(4-hydroxy-3,5-dimethylphenyl)-7-(morpholinomethyl)isoquinolin-1(2H)-one;
2-hydroxy-7-(4-hydroxy-3,5-dimethylphenyl)-4-methoxy-1,6-naphthyridin-5(6H)-one;
2-(4-hydroxy-3,5-dimethylphenyl)-5,7-dimethoxyquinazolin-4(3H)-one;
3-(4-(2-hydroxy-2-methylpropoxy)-3,5-dimethylphenyl)-6,8-dimethoxy-isoquinolin-1(2H)-one;
6,8-dimethoxy-3-(4-hydroxy-3,5-dimethylphenyl)-2H-1,2-benzothiazine-1,1-dioxide;
7-(4-hydroxy-3,5-dimethylphenyl)-2,4-dimethoxy-1,6-naphthyridin-5(6H)-one;
3-(3,5-dimethyl-4-(2-morpholinoethoxy)phenyl)-6,8-dimethoxyisoquinolin-1(2H)-one;
2-(4-(2-hydroxyethoxy)-3,5-dimethylphenyl)-5,7-dimethoxyquinazolin-4(3H)-one);
2-(3,5-dimethyl-4-(2-morpholinoethoxy)phenyl)-5,7-dimethoxyquinazolin-4(3H)-one;
3-(3,5-dimethyl-4-(2-(4-methylpiperazin-1-yl)ethoxy)phenyl)-6,8-dimethoxy-isoquinolin-1(2H)-one;
2-(4-hydroxyphenyl)-5,7-dimethoxyquinazolin-4(3H)-one;
2-(4-(5,7-dimethoxy-4-oxo-3,4-dihydroquinazolin-2-yl)phenoxy)acetic acid;
2-(3,5-di-tert-butyl-4-hydroxyphenyl)-5,7-dimethoxyquinazolin-4(3H)-one;
2-(3,5-dimethoxyphenyl)-5,7-dimethoxyquinazolin-4(3H)-one;
5,7-dimethoxy-2-(4-methoxyphenyl)quinazolin-4(3H)-one;
2-(4-hydroxy-3-methoxyphenyl)-5,7-dimethoxyquinazolin-4(3H)-one;
2-(3-chloro-4-hydroxyphenyl)-5,7-dimethoxyquinazolin-4(3H)-one;
2-(4-(dimethylamino)naphthalen-1-yl)-5,7-dimethoxyquinazolin-4(3H)-one;
2-(4-(5,7-dimethoxy-4-oxo-3,4-dihydroquinazolin-2-yl)phenoxy)acetamide;
2-(2,3-dihydrobenzo[b][1,4]dioxin-6-yl)-5,7-dimethoxyquinazolin-4(3H)-one;
5,7-dimethoxy-2-(4-(4-methylpiperazin-1-yl)phenyl)quinazolin-4(3H)-one;
2-(4-(bis(2-hydroxyethyl)amino)phenyl)-5,7-dimethoxyquinazolin-4(3H)-one;
5,7-dimethoxy-2-(4-morpholinophenyl)quinazolin-4(3H)-one;
7-(3,5-dimethyl-4-(2-morpholinoethoxy)phenyl)-2,4-dimethoxy-1,6-naphthyridin-5(6H)-one;
3-(4-(5,7-dimethoxy-4-oxo-3,4-dihydroquinazolin-2-yl)phenyl)propanoic acid;
2-(3-chloro-4-(2-hydroxyethoxy)phenyl)-5,7-dimethoxyquinazolin-4(3H)-one;
2-(4-(2-hydroxyethoxy)-3-methoxyphenyl)-5,7-dimethoxyquinazolin-4(3H)-one;
5,7-dimethoxy-2-(4-((4-methylpiperazin-1-yl)methyl)phenyl)quinazolin-4(3H)-one;
N-(4-(5,7-dimethoxy-4-oxo-3,4-dihydroquinazolin-2-yl)-2,6-dimethylphenyl)-2-hydroxyacetamide;
7-(4-(2-hydroxyethoxy)-3,5-dimethylphenyl)-2,4-dimethoxy-1,6-naphthyridin-5(6H)-one;
2,4-dimethoxy-7-(4-methoxy-3,5-dimethylphenyl)-1,6-naphthyridin-5(6H)-one;
2-(4-(5,7-dimethoxy-4-oxo-3,4-dihydroquinazolin-2-yl)-2,6-dimethylphenoxy)acetic acid;
N-(4-(5,7-dimethoxy-4-oxo-3,4-dihydroquinazolin-2-yl)phenyl)-2-hydroxyacetamide;
5,7-dimethoxy-2-(4-(morpholinomethyl)phenyl)quinazolin-4(3H)-one;
2-(4-((4-ethylpiperazin-1-yl)methyl)phenyl)-5,7-dimethoxyquinazolin-4(3H)-one;
2-(4-hydroxy-3,5-dimethylphenyl)-5,7-dimethoxypyrido[2,3-d]pyrimidin-4(3H)-one;
5,7-dimethoxy-2-(4-methoxy-3-(morpholinomethyl)phenyl)quinazolin-4(3H)-one;
2-(4-(2-hydroxyethoxy)-3,5-dimethylphenyl)-5,7-dimethoxypyrido[2,3-d]pyrimidin-4(3H)-one;
2-(3,5-dimethyl-4-(2-(pyrrolidin-1-yl)ethoxy)phenyl)-5,7-dimethoxy-quinazolin-4(3H)-one;
7-(4-hydroxy-3,5-dimethylphenyl)-2,4-diisopropoxy-1,6-naphthyridin-5(6H)-one;
2-(4-hydroxy-3-(2-hydroxyethyl)phenyl)-5,7-dimethoxyquinazolin-4(3H)-one;
2-(4-(2-hydroxyethoxy)-3,5-dimethylphenyl)-5,7-dimethylquinazolin-4(3H)-one;
2-(4-(bis(2-hydroxyethyl)amino)phenyl)-5,7-dimethoxypyrido[2,3-d]pyrimidin-4(3H)-one;
5,7-dimethoxy-2-(4-(2-methoxyethoxy)-3,5-dimethylphenyl)quinazolin-4(3H)-one;
5,7-dichloro-2-(4-(2-hydroxyethoxy)-3,5-dimethylphenyl)quinazolin-4(3H)-one;
5,7-dimethoxy-2-(4-methoxy-3,5-dimethylphenyl)quinazolin-4(3H)-one;
2-(4-amino-3,5-dimethylphenyl)-5,7-dimethoxyquinazolin-4(3H)-one;
2-(3,5-dimethylphenyl)-5,7-dimethoxyquinazolin-4(3H)-one;
(E)-N′-(4-(5,7-dimethoxy-4-oxo-3,4-dihydroquinazolin-2-yl)-2,6-dimethyl-phenyl)-N,N-dimethylformimidamide;
2-(4-(benzyloxy)-3,5-dimethylphenyl)-5,7-dimethoxyquinazolin-4(3H)-one;
2-(4-(2-hydroxyethoxy)-3-methylphenyl)-5,7-dimethoxyquinazolin-4(3H)-one;
2-(4-hydroxy-3-methylphenyl)-5,7-dimethoxyquinazolin-4(3H)-one;
N1-(2-(4-(5,7-dimethoxy-4-oxo-3,4-dihydroquinazolin-2-yl)-2,6-dimethyl-phenoxy)ethyl)-N2-methylphthalamide;
2-(4-(2-aminoethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethyl-phenoxy)ethyl)-4-methoxybenzenesulfonamide;
4-chloro-N-(2-(4-(5,7-dimethoxy-4-oxo-3,4-dihydroquinazolin-2-yl)-2,6-dimethylphenoxy)ethyl)benzenesulfonamide;
N-(2-(4-(5,7-dimethoxy-4-oxo-3,4-dihydroquinazolin-2-yl)-2,6-dimethyl-phenoxy)ethyl)methanesulfonamide;
2-(4-(5,7-dimethoxy-4-oxo-3,4-dihydroquinazolin-2-yl)-2-methoxy-phenoxy)acetic acid;
2-(4-(5,7-Dimethoxy-4-oxo-3,4-dihydroquinazolin-2-yl)-2,6-dimethyl-phenoxy)ethyl propylcarbamate;
2-(4-(5,7-Dimethoxy-4-oxo-3,4-dihydroquinazolin-2-yl)-2,6-dimethyl-phenoxy)ethyl methylcarbamate;
N-(2-(4-(5,7-Dimethoxy-4-oxo-3,4-dihydroquinazolin-2-yl)-2,6-dimethyl-phenoxy)ethyl)-4-methylbenzamide;
2-(4-(5,7-dimethoxy-4-oxo-3,4-dihydroquinazolin-2-yl)-2,6-dimethyl-phenoxy)ethyl cyclohexylcarbamate;
N-(2-(4-(5,7-dimethoxy-4-oxo-3,4-dihydroquinazolin-2-yl)-2,6-dimethyl-phenoxy)ethyl)benzenesulfonamide;
N-(2-(4-(5,7-dimethoxy-4-oxo-3,4-dihydroquinazolin-2-yl)-2,6-dimethyl-phenoxy)ethyl)-4-methylbenzenesulfonamide;
N-(2-(4-(5,7-dimethoxy-4-oxo-3,4-dihydroquinazolin-2-yl)-2,6-dimethyl-phenoxy)ethyl)-4-methoxybenzamide;
N-(2-(4-(5,7-dimethoxy-4-oxo-3,4-dihydroquinazolin-2-yl)-2,6-dimethyl-phenoxy)ethyl)acetamide;
N-(2-(4-(5,7-dimethoxy-4-oxo-3,4-dihydroquinazolin-2-yl)-2,6-dimethyl-phenoxy)ethyl)benzamide;
N-(2-(4-(5,7-dimethoxy-4-oxo-3,4-dihydroquinazolin-2-yl)-2,6-dimethyl-phenoxy)ethyl)isobutyramide;
1-(2-(4-(5,7-dimethoxy-4-oxo-3,4-dihydroquinazolin-2-yl)-2,6-dimethyl-phenoxy)ethyl)-3-methyl urea;
1-(2-(4-(5,7-dimethoxy-4-oxo-3,4-dihydroquinazolin-2-yl)-2,6-dimethyl-phenoxy)ethyl)-3-(4-methoxyphenyl)urea;
1-(2-(4-(5,7-dimethoxy-4-oxo-3,4-dihydroquinazolin-2-yl)-2,6-dimethyl-phenoxy)ethyl)-3-phenylurea;
3-(2-(4-(5,7-dimethoxy-4-oxo-3,4-dihydroquinazolin-2-yl)-2,6-dimethyl-phenoxy)ethyl)-1,1-dimethylurea;
and stereoisomers, tautomers, pharmaceutically acceptable salts, and hydrates thereof.

16. The method of claim 1, wherein the therapeutically effective amount of the compound of Formula I or tautomer, stereoisomer, pharmaceutically acceptable salt, or hydrate thereof is administered with a pharmaceutically acceptable carrier in a pharmaceutically acceptable composition.

17. The method of claim 2, wherein the therapeutically effective amount of the compound of Formula II or tautomer, stereoisomer, pharmaceutically acceptable salt, or hydrate thereof is administered with a pharmaceutically acceptable carrier in a pharmaceutically acceptable composition.

18. The method of claim 1, wherein the compound of Formula I is administered to treat or prevent a disease or disorder that responds to a BET inhibitor, wherein the disease or disorder is selected from cancer, autoimmune or inflammatory diseases or conditions, diseases or disorders caused by bacterial or viral infection.

19. The method of claim 2, wherein the compound of Formula II is administered to treat or prevent a disease or disorder that responds to a BET inhibitor, wherein the disease or disorder is selected from cancer, autoimmune or inflammatory diseases or conditions, diseases or disorders caused by bacterial or viral infection.

20. The method of claim 18, wherein the disease or disorder is a cancer selected from cancers that exhibit c-myc overexpression, cancers that overexpress n-myc, cancers that that rely on the recruitment of p-TEFb to regulate activated oncogenes, Burkitt's lymphoma, acute myelogenous leukemia, multiple myeloma, aggressive human medulloblastoma, hematological, epithelial cancers, lung cancers, breast cancers, colon carcinomas, midline carcinomas, mesenchymal tumors, hepatic tumors, renal tumors, and neurological tumors.

21. The method of claim 1, wherein, the compound of Formula I or tautomer, stereoisomer, pharmaceutically acceptable salt, or hydrate thereof induces apoptosis in cancer cells by decreasing expression of the anti-apoptosis gene Bcl2.

22. The method of claim 1, wherein the compound of Formula I is administered in combination with another anti-cancer agent selected from the group consisting of bortezomib, thalidomide, dexamethasone, 5-azacitidine, decitabine, vorinostat, cyclophosphamide, a PI3K or mTOR inhibitor, rapamycin or a rapamycin analog, a gamma secretase inhibitor, an AMPK inducer, metformin, phenformin, an ornithine decarboxylase inhibitor, and difluoromethylornithine.
